(19) United States
(12) Patent Application Publication
(10) Pub. No.: US 2024/0300957 A1
(43) Pub. Date: Sep. 12, 2024

(54) TETRAZOLE-SUBSTITUTED ETHYL-SULFONE / SULFOXIMINE-PYRIDINE DERIVATIVES

(71) Applicants: Boehringer Ingelheim Vetmedica GmbH, Ingelheim am Rhein (DE); NIHON NOHYAKU CO., LTD., Tokyo (JP)

(72) Inventors: Bart Herlé, Mittelbiberach (DE); Hannes Fiepko KOOLMAN, Mittelbiberach (DE)

(21) Appl. No.: 18/582,375

(22) Filed: Feb. 20, 2024

(30) Foreign Application Priority Data
Feb. 23, 2023 (EP) 23158088.7

(51) Int. Cl. C07D 487/04; A61K 31/4439; A61K 31/444; A61K 31/5025; A61P 33/14; C07D 401/14; C07D 471/04

(52) U.S. Cl. CPC C07D 487/04; A61K 31/4439; A61K 31/444; A61K 31/5025; A61P 33/14; C07D 401/14; C07D 471/04

(57) ABSTRACT

The present invention relates to tetrazole substituted ethylsulfone/sulfoximine-pyridine derivatives of formula (I), (Ia) or (Ib)

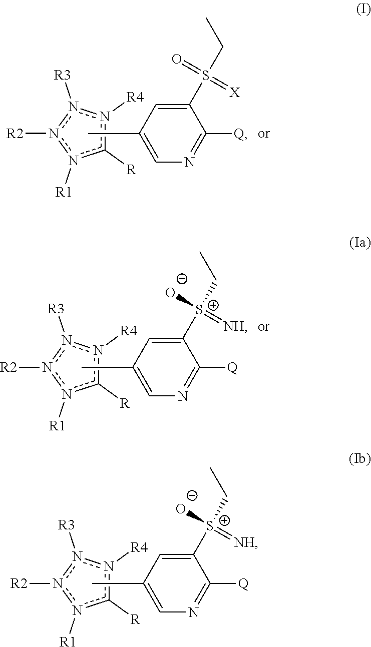

wherein the variables are as defined in the description and/or in the claims, which can be used as antiparasitic agents for the treatment, prevention and/or control of parasitic infections and/or infestations in/on animals as well as as crop protection agents.

### CROSS REFERENCE TO RELATED APPLICATIONS

This application claims the benefit of priority to European Patent Application No. 23 158 088.7 filed on Feb. 23, 2023, which is incorporated herein by reference in its entirety.

### INCORPORATION BY REFERENCE

All references cited herein, are incorporated by reference herein, in their entirety.

### BACKGROUND OF THE INVENTION

### Field of the Invention

The present invention relates to the field of animal health and crop protection, in particular to new tetrazole substituted ethylsulfone/sulfoximine-pyridine derivatives according to formula (I), (Ia) or (Ib) as antiparasitic and/or crop protection compounds as well as pharmaceutical compositions containing the same, and methods of using the same as antiparasitic agents for the treatment, prevention and/or control of parasitic infections and/or infestations in animals as well as as crop protection agents.

### Background and Description of the Related Art

Animals, such as mammals and birds are often susceptible to parasite infestations/infections. These parasites may be ectoparasites, such as insects and arachnids, and endoparasites such as filariae and other roundworms. Domesticated animals, such as cats and dogs, are often infested with one or more of the following ectoparasites: fleas (e.g. Ctenocephalides spp., such as Ctenocephalides felis and the like), ticks (e.g. Rhipicephalus spp., Ixodes spp., Dermacentor spp., Amblyoma spp., and the like), mites (e.g. Demodex spp., Sarcoptes spp., Otodectes spp., and the like), lice (e.g. Trichodectes spp., Cheyletiella spp., Linognathus spp. and the like), mosquitoes (Aedes spp., Culex spp., Anopheles spp. and the like) and flies (Hematobia spp., Musca spp., Stomoxys spp., Dematobia spp., Cocliomyia spp. and the like).

Fleas are a particular problem because not only do they adversely affect the health of the animal or human, but they also cause a great deal of psychological stress. Moreover, fleas are also vectors of pathogenic agents in animals and humans, such as dog tapeworm (Dipylidium caninum).

Similarly, ticks are also harmful to the physical and psychological health of the animal or human. However, the most serious problem associated with ticks is that they are the vector of pathogenic agents in both humans and animals. Major diseases which are caused by ticks include borrelioses (Lyme disease caused by Borrelia burgdorferi), babesioses (or piroplasmoses caused by Babesia spp.) and rickettsioses (also known as Rocky Mountain spotted fever). Ticks also release toxins which cause inflammation or paralysis in the host. Occasionally, these toxins are fatal to the host.

Likewise, farm animals are also susceptible to parasite infestations. For example, cattle are affected by a large number of parasites. A parasite which is very prevalent among farm animals is the tick genus Boophilus, especially those of the species microplus (cattle tick), decoloratus and annulatus. Ticks, such as Rhipicephalus microplus (formerly Boophilus microplus), are particularly difficult to control because they live in the pasture where farm animals graze.

Currently available insecticidal and acaricidal treatments for animals do not always demonstrate good activity, good speed of action, or a long duration of action. Most treatments contain hazardous chemicals that can have serious consequences, including neurotoxicity and lethality from accidental ingestion. Persons applying these agents are generally advised to limit their exposure. Pet collars and tags have been utilized to overcome some problems, but these are susceptible to chewing, ingestion, and subsequent toxicological effects to the animal. Thus, current treatments achieve varying degrees of success, which depend partly on toxicity, method of administration, and efficacy. Additionally, some currently available agents are becoming ineffective due to parasitic resistance.

Despite the availability of effective, broad spectrum antiparasitics, there remains a need for safer and more convenient, efficacious, and environmentally friendly products that will overcome the ever-present threat of resistance development. There is a need for improved antiparasitics, and in particular there is a need for improved insecticides and acaricides, particularly for use in animal health. Furthermore, there is a need for improved topical and oral products with convenient administration. Still further, there is a need for improved compositions which contains one or more active antiparasitics, which can be used to effectively treat against parasites. Such improvements would be particularly useful for the treatment of animals including companion animals (e.g., cats, dogs, llamas, and horses) and livestock (e.g., cattle, bison, swine, sheep, deer, elk, and goats).

Further prior art is as follows:
WO 2016/030229 discloses compounds and the agrochemically acceptable salts, stereoisomers, enantiomers, tautomers and N-oxides of those compounds, which can be used as insecticides and can be prepared in a manner known per se.
WO 2016/059145 discloses compounds and the agrochemically acceptable salts salts, stereoisomers, enantiomers, tautomers and N-oxides of those compounds, which can be used as insecticides and can be prepared in a manner known per se.
WO 2016/071214 discloses polycyclic compounds and the agrochemically acceptable salts, stereoisomers, enantiomers, tautomers and N-oxides of those compounds, which can be used as insecticides and can be prepared in a manner known per se.
WO 2018/095795 discloses polycyclic compounds and the agrochemically acceptable salts, stereoisomers, enantiomers, tautomers and N-oxides of those compounds, which can be used as insecticides and can be prepared in a manner known per se.
WO 2020/182577 discloses compounds and agrochemical compositions which comprise such compounds as well as preparation of these compositions, and the use of the compounds or compositions in agriculture or horticulture for combating, preventing or controlling animal pests, including arthropods and in particular insects, nematodes, molluscs or representatives of the order Acarina.

Starting from the structurally closest prior art, i.e. the three nitrogen containing triazole compounds “P106” and “P110” of WO 2016/030229, there is a need for improved antiparasitics that are for example more potent/efficacious against in particular ectoparasites fleas and/or ticks.

### SUMMARY OF THE INVENTION

The present invention solves the problems inherent in the related art and provides a distinct advance in the state of the art.

The present invention concerns a compound of formula (I) or formula (Ia) or formula (Ib)

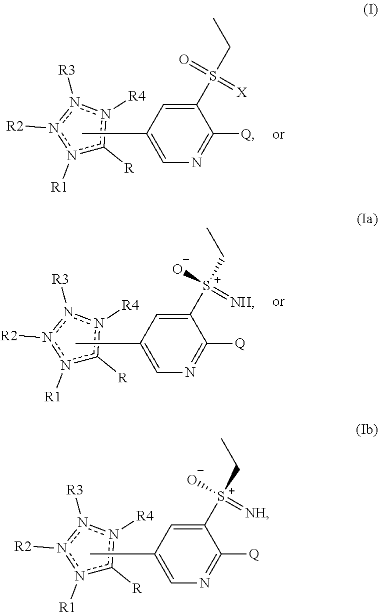

wherein:
X, if present, is independently O or NH, preferably O;

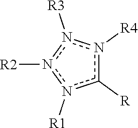

is attached to the central pyridine ring via the C atom or any N atom of the four nitrogen atoms containing five-membered ring system; preferably via the C atom;
R is independently absent, hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈- cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkoxy, such as methoxy, ethoxy, propoxy, C₁-C₆-haloalkoxy, such as O—CF₃, C₁-C₆-alkyl-C₁-C₆-alkoxy, such as CH₂—O—CH₃;
R1, R2, R3, R4 are each independently absent, hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃;
Q is selected from the group consisting of: Q1, Q2, Q3, wherein:
Q1 is an unsubstituted or substituted monocyclic, preferably 5- or 6-membered, heteroaryl or heterocyclyl containing one, two or three heteroatoms, which are each independently selected from the group consisting of N, O, S, S(O), S(O)₂; wherein preferably Q1 if substituted is substituted with one, two, three or four substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH₂, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂—C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, ═O, S(O)NRₓR_{y}, such as S(O)NH—CF₃;
Q2 is an unsubstituted or substituted fused bicyclic, preferably 9- or 10-membered, heteroaryl or heterocyclyl containing one, two, three, four or five heteroatoms, which are each independently selected from the group consisting of N, O, S, S(O), S(O)₂; wherein preferably Q2 if substituted is substituted with one, two, three, four, five or six substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH₂, C₁-C₆-haloalkyl, such as CF₃, C_{2F}s, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂—C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, S(O)ᵣ—(C₁-C₆-alkyl), such as S-methyl; S(O)-methyl, S(O)₂-methyl; S(O)ₛ—(C₁-C₆-haloalkyl), such as S—CF₃, S(O)—CF₃, S(O)₂—CF₃, ═O, S(O)NRₓR_{y}, such as S(O)NH—CF₃;
Q3 is an unsubstituted or substituted fused tricyclic, preferably 12- or 13-membered, heteroaryl or heterocyclyl containing one, two, three, four, five, six or seven heteroatoms, which are each independently selected from the group consisting of N, O, S, S(O), S(O)₂; wherein preferably Q3 if substituted is substituted with one, two, three, four, five, six, seven or eight substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH₂, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂—C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, S(O)ᵣ—(C₁-C₆-alkyl), such as S-methyl; S(O)-methyl, S(O)₂-methyl; S(O)ₛ—(C₁-C₆-haloalkyl), such as S—CF₃, S(O)—CF₃, S(O)₂—CF₃, ═O, S(O)NRₓR_{y}, such as S(O)NH—CF₃;
Rₓ, R_{y} in each occurrence are independently selected from the group consisting of: hydrogen, halogen, such as F, Cl, Br, I, C₁-C₆-alkyl, such as methyl, ethyl, C₁-C₆-haloalkyl, such as CF₃;
r, s in each occurrence independently are 0, 1 or 2;
the dashed bond ( [symbol] ) signifies a single or double bond;
optionally, wherein at least one nitrogen atom is an N-oxide,
optionally, but preferably in case at least one nitrogen atom is an N-oxide, with the first proviso that no nitrogen atom in the four nitrogen atoms containing five-membered ring system is an N-oxide;
optionally, but preferably in case at least one nitrogen atom is an N-oxide, with the second proviso that if X is present and is NH, the NH nitrogen atom is not an N-oxide;
optionally, but preferably in case at least one nitrogen atom is an N-oxide, with the third proviso that, if possible, the at least one nitrogen atom being an N-oxide is not present in any of the radicals R, R1, R2, R3, R4;
optionally, but preferably in case at least one nitrogen atom is an N-oxide, with the fourth proviso that, if possible, and if any of Q being Q1, Q2 or Q3 is substituted, the at least one nitrogen atom being an N-oxide is not present in any potential substituents of any of Q being Q1, Q2 or Q3;
optionally, but preferably in case at least one nitrogen atom is an N-oxide, the at least one nitrogen atom being an N-oxide comprises at least one nitrogen atom within a six-membered heterocyclic moiety containing one or more nitrogen atoms of any one of Q being Q1, Q2 or Q3;
or a salt thereof.

The present invention also concerns a compound of formula (I), (Ia) or (Ib) as herein disclosed and/or claimed, wherein Q is Q1, Q2 or Q3, which are independently selected from the group consisting of:

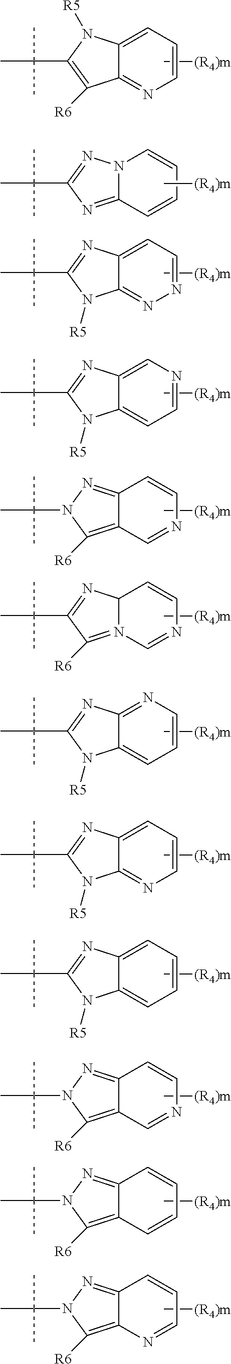

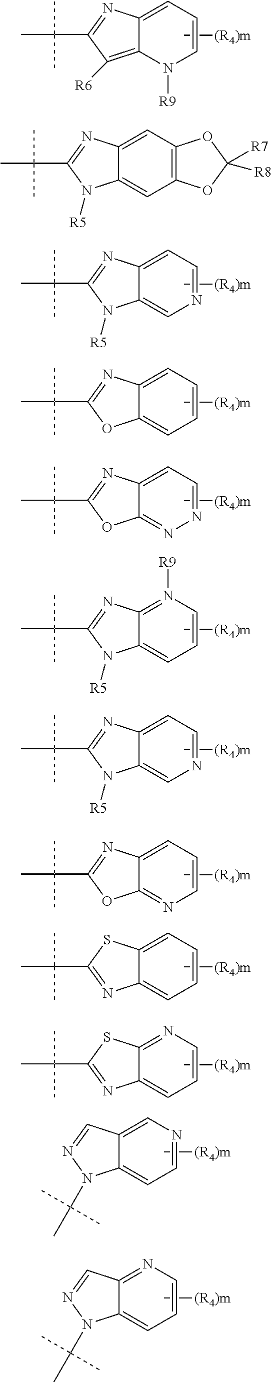

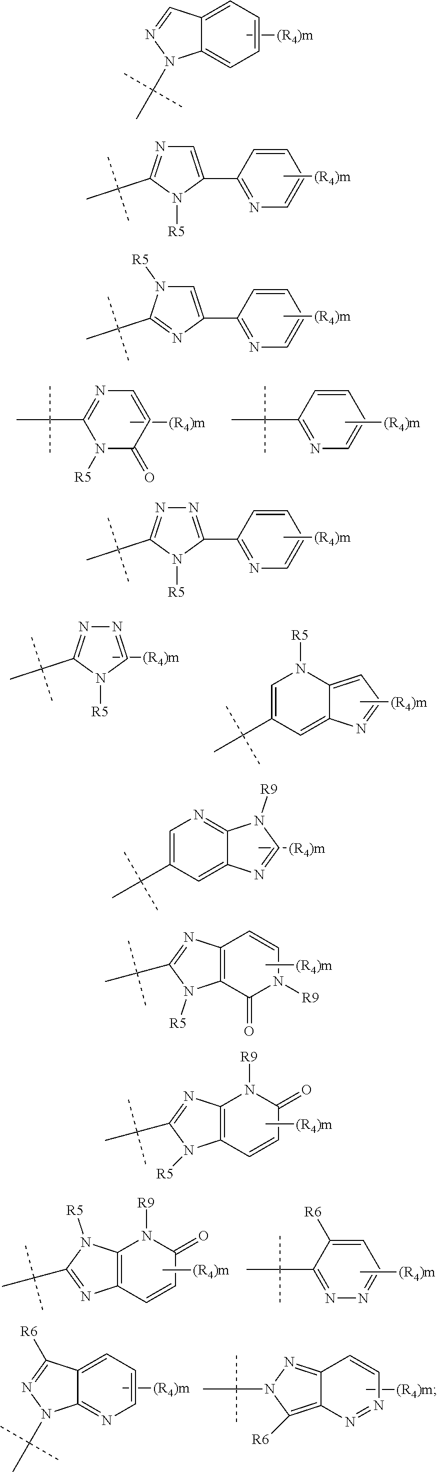

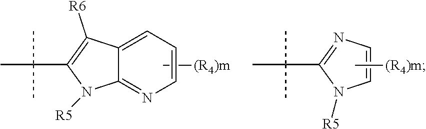

wherein:
R₄ is C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom;
R5, R9 are each independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃;
R6, R7, R8 are each independently hydrogen, halogen, such as F, Cl, Br, I;
m is 0, 1 or 2;
p, q in each occurrence independently are 0, 1 or 2;
or a salt thereof.

The present invention further concerns a compound of formula (I), (Ia) or (Ib) as herein disclosed and/or claimed, wherein:

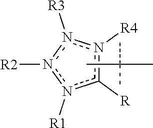

is independently selected from the group consisting of:

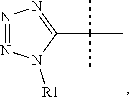

wherein R1 is as defined as herein disclosed and/or claimed, but is present, such as R1 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R2, R3, R4 are all absent;

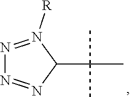

wherein R is as defined as herein disclosed and/or claimed, but is present, such as R is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R1, R2, R3, R4 are all absent;

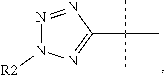

wherein R2 is as defined as herein disclosed and/or claimed, but is present, such as R2 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R1, R3, R4 are all absent;
or a salt thereof.

The present invention further concerns a compound of formula (I), (Ia) or (Ib), preferably as herein disclosed and/or claimed,

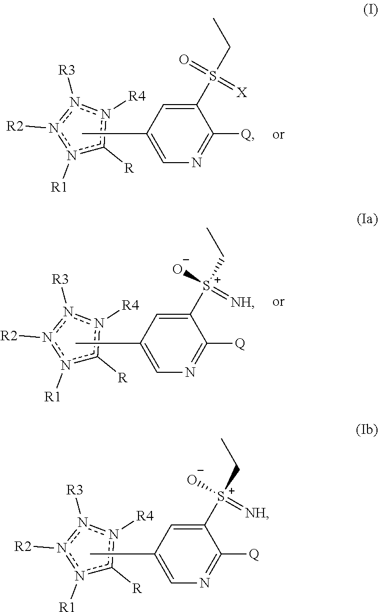

wherein:
X, if present, is independently O or NH, preferably O;
and

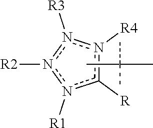

is independently selected from the group consisting of:

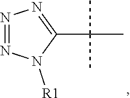

R1 is present and is independently hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C3-C8-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃; in particular R1 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R2, R3, R4 are all absent;
or

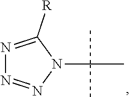

R is present and is independently hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkoxy, such as methoxy, ethoxy, propoxy, C₁-C₆-haloalkoxy, such as O—CF₃, C₁-C₆-alkyl-C₁-C₆-alkoxy, such as CH₂—O—CH₃; in particular R is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R1, R2, R3, R4 are all absent;
or

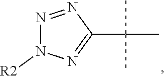

R2 is present and is independently hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C3-C8-cycloalkyl, such as cyclopropyl, C3-C8-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃; in particular R2 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R1, R3, R4 are all absent;
and Q is Q1, Q2 or Q3, which are independently selected from the group consisting of:

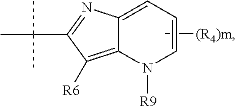

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CF₃” or “CHF₂” or “CF₂—CH₃”;
R9 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R9 is “CHF₂” or “CH₂—CHF₂” or “ethyl” or “CH₂-cyclopropyl”,
R6 is independently hydrogen, halogen, such as F, Cl, Br, I; in particular R6 is “hydrogen” or “F”;
m is 0, 1 or 2; in particular m is 1;
p, q in each occurrence independently are 0, 1 or 2;
or

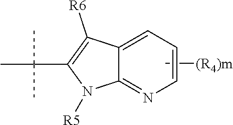

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CF₂CF₃” or “CHF₂” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “hydrogen” or “methyl”;
R6 is independently hydrogen, halogen, such as F, Cl, Br, I; in particular R6 is “hydrogen”;
m is 0, 1 or 2; in particular m is 1;
p, q in each occurrence independently are 0, 1 or 2;
or

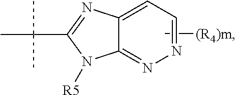

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CHF₂” or “CF₂-cyclopropyl” or “CF₂—CF₃” or “CF₂—CH₂—CH₃” or “CF₃” or “S—CF₃” or “S(O)₂—CF₃” or “O—CH₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “methyl”;
m is 0, 1 or 2; in particular m is 1;
p, q in each occurrence independently are 0, 1 or 2;
or

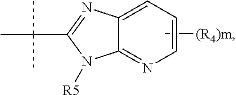

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CHF₂” or “CF₂—CF₃” or “CF₂-cyclopropyl” or “CF₂—CH₂—CH₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “methyl”;
m is 0, 1 or 2; in particular m is 1;
p, q in each occurrence independently are 0, 1 or 2;
or

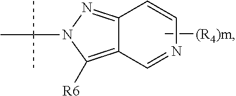

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CF₃” or “CHF₂” or “CF₂—CF₃” or “S—CF₃” or “S(O)—CF₃” or “S(O)₂—CF₃” or “CF₂—CH₃”;
R6 is independently hydrogen, halogen, such as F, Cl, Br, I; in particular R6 is “hydrogen” or “F”;
m is 0, 1 or 2; in particular m is 1;
p, q in each occurrence independently are 0, 1 or 2;
or

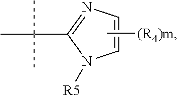

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “C(O)NH—CH₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “methyl”;
m is 0, 1 or 2; in particular m is 1;
p, q in each occurrence independently are 0, 1 or 2;
or

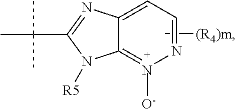

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CHF₂” or “CF₂-cyclopropyl” or “CF₂—CF₃” or “CF₂—CH₂—CH₃” or “S—CF₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “methyl”;
m is 0, 1 or 2; in particular m is 1;
p, q in each occurrence independently are 0, 1 or 2;
or

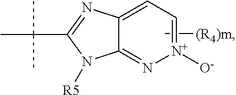

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CHF₂” or “CF₂-cyclopropyl” or “CF₂—CF₃” or “CF₂—CH₂—CH₃” or “S—CF₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “methyl”;
m is 0, 1 or 2; in particular m is 1;
p, q in each occurrence independently are 0, 1 or 2;
or

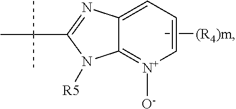

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CHF₂” or “CF₂—CF₃” or “CF₂-cyclopropyl” or “CF₂—CH₂—CH₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; in particular R5 is “methyl”;
m is 0, 1 or 2; in particular m is 1;
p, q in each occurrence independently are 0, 1 or 2;
or

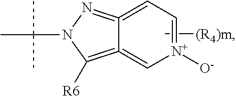

wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂—C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; in particular R₄ is “CF₃” or “CHF₂” or “CF₂—CF₃” or “S—CF₃” or “S(O)—CF₃” or “S(O)₂—CF₃” or “CF₂—CH₃”;
R6 is independently hydrogen, halogen, such as F, Cl, Br, I; in particular R6 is “hydrogen” or “F”;
m is 0, 1 or 2; in particular m is 1;
p, q in each occurrence independently are 0, 1 or 2;
and/or
wherein optionally, if any, the at least one nitrogen atom being an N-oxide comprises the one of the central pyridine moiety selected from the group consisting of formula (II) or formula (IIa) or formula (IIb):

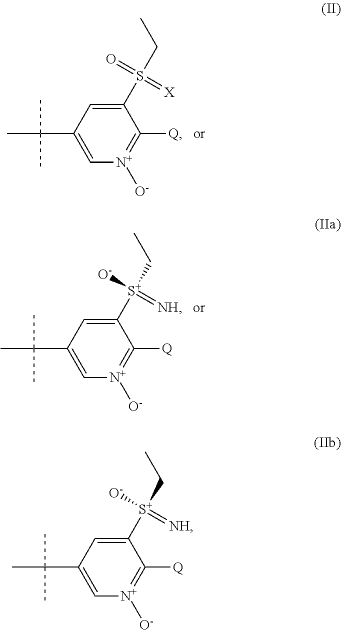

wherein X, if present, is independently O or NH, preferably O; and formulae (II), (IIa) and (JIb) are only part of the corresponding formulae (I), (Ia) and (Ib), respectively;
and
wherein the dashed bond ( [symbol] ) signifies a single or double bond;
optionally, wherein at least one nitrogen atom is an N-oxide,
optionally, but preferably in case at least one nitrogen atom is an N-oxide, with the first proviso that no nitrogen atom in the four nitrogen atoms containing five-membered ring system is an N-oxide;
optionally, but preferably in case at least one nitrogen atom is an N-oxide, with the second proviso that if X is present and is NH, the NH nitrogen atom is not an N-oxide;
optionally, but preferably in case at least one nitrogen atom is an N-oxide, with the third proviso that, if possible, the at least one nitrogen atom being an N-oxide is not present in any of the radicals R, R1, R2, R3, R4;
optionally, but preferably in case at least one nitrogen atom is an N-oxide, with the fourth proviso that, if possible, and if any of Q being Q1, Q2 or Q3 is substituted, the at least one nitrogen atom being an N-oxide is not present in any potential substituents of any of Q being Q1, Q2 or Q3;
optionally, but preferably in case at least one nitrogen atom is an N-oxide, the at least one nitrogen atom being an N-oxide comprises at least one nitrogen atom within a six-membered heterocyclic moiety containing one or more nitrogen atoms of any one of Q being Q1, Q2 or Q3;
or a salt thereof.

The present invention further concerns a compound of formula (I), (Ia) or (Ib), preferably as herein disclosed and/or claimed, wherein the compound is selected from the group consisting of:

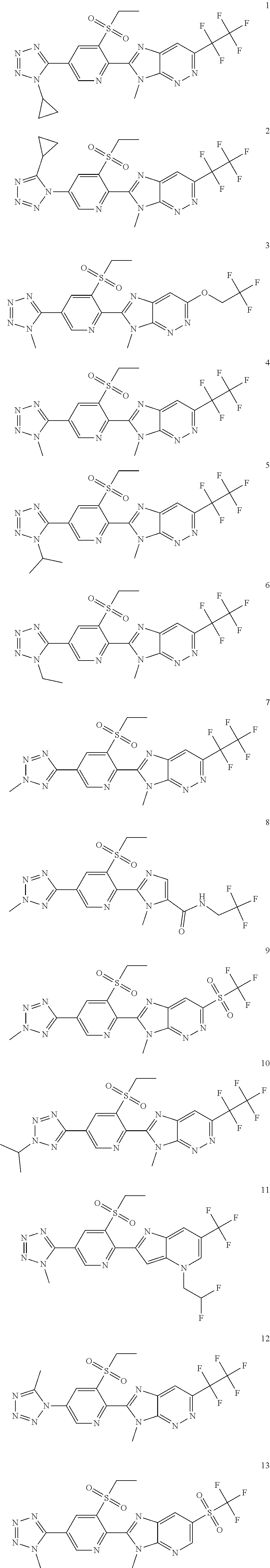

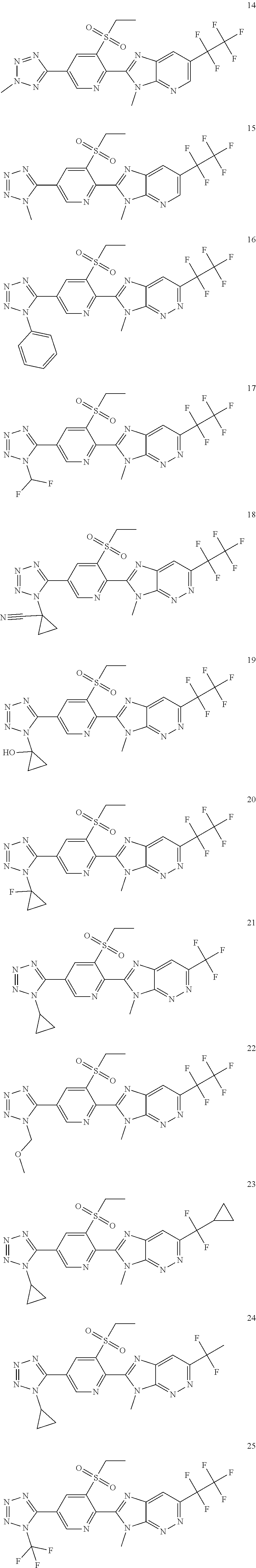

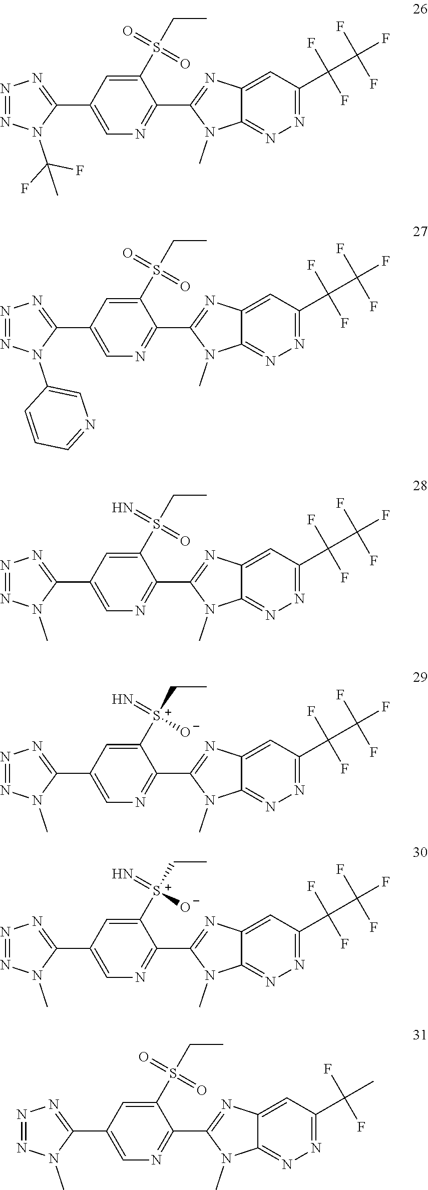

or a pharmaceutically acceptable salt thereof.

The present invention further concerns a compound of formula (I), (Ia) or (Ib), preferable as herein disclosed and/or claimed, wherein the compound is selected is selected from the group consisting of:

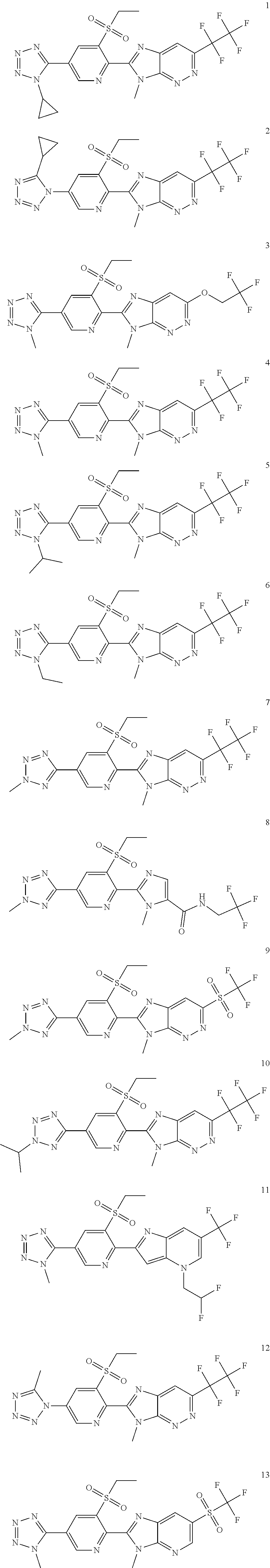

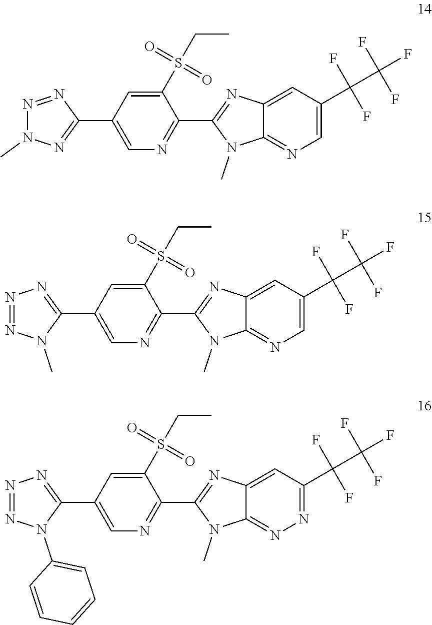

or a pharmaceutically acceptable salt thereof.

The present invention further concerns a pharmaceutical composition comprising one or more compound(s) of formula (I), (la) and/or (Ib) as herein disclosed and/or claimed or a pharmaceutically acceptable salt thereof and one or more pharmaceutically acceptable excipient(s).

The present invention further concerns a pharmaceutical composition consisting essentially of one or more compound(s) of formula (I), (la) and/or (Ib) as herein disclosed and/or claimed or a pharmaceutically acceptable salt thereof and one or more pharmaceutically acceptable excipient(s).

The present invention further concerns a pharmaceutical composition consisting of one or more compound(s) of formula (I), (Ia) and/or (Ib) as herein disclosed and/or claimed or a pharmaceutically acceptable salt thereof and one or more pharmaceutically acceptable excipient(s).

The present invention further concerns a pharmaceutical composition comprising one or more compound(s) of formula (I), (Ta) and/or (Tb) as herein disclosed and/or claimed or a pharmaceutically acceptable salt thereof, one or more additional pharmaceutically active agent(s), and one or more pharmaceutically acceptable excipient(s).

The present invention further concerns a pharmaceutical composition consisting essentially of one or more compound(s) of formula (I), (Ta) and/or (Tb) as herein disclosed and/or claimed or a pharmaceutically acceptable salt thereof, one or more additional pharmaceutically active agent(s), and one or more pharmaceutically acceptable excipient(s).

The present invention further concerns a pharmaceutical composition consisting of one or more compound(s) of formula (I), (Ta) and/or (Tb) as herein disclosed and/or claimed or a pharmaceutically acceptable salt thereof, one or more additional pharmaceutically active agent(s), and one or more pharmaceutically acceptable excipient(s).

The present invention further concerns a compound of formula (I), (Ta) or (Tb) as herein disclosed and/or claimed or a pharmaceutically acceptable salt thereof or a pharmaceutical composition as herein disclosed and/or claimed for use as a medicament, preferably for use as an antiparasitic medicament. A corresponding use of a compound of formula (I), (Ta) or (Ib) as herein disclosed and/or claimed or a pharmaceutically acceptable salt thereof or a pharmaceutical composition as herein disclosed and/or claimed for the preparation of a medicament, preferably an antiparasitic medicament, are also intended to be comprised by the present invention.

The present invention further concerns a compound of formula (I), (Ta) or (Ib) as herein disclosed and/or claimed or a pharmaceutically acceptable salt thereof or a pharmaceutical composition as herein disclosed and/or claimed for use in a method of treatment, prevention and/or control of a parasitic infection and/or infestation in an animal, preferably of an ectoparasitic infection and/or infestation in an animal, more preferably of an infection and/or infestation of fleas and/or ticks in/on an animal. A corresponding method of treatment, prevention and/or control of a parasitic infection and/or infestation in an animal, comprising administering an effective amount of compound of formula (I), (Ta) or (Ib) as herein disclosed and/or claimed or a pharmaceutically acceptable salt thereof or a pharmaceutical composition as herein disclosed and/or claimed to such animals, as well as the corresponding use of compound of formula (I), (Ta) or (Tb) as herein disclosed and/or claimed or a pharmaceutically acceptable salt thereof or a pharmaceutical composition as herein disclosed and/or claimed for the preparation of a medicament for the treatment, prevention and/or control of a parasitic infection and/or infestation in/on an animal, are also intended to be comprised by the present invention.

The compounds of formula (I), (Ta) or (Tb) as herein disclosed and/or claimed or a pharmaceutically acceptable salt thereof are advantageously more potent/efficacious against the ectoparasites fleas and/or ticks, as compared to the structurally closest prior art, that is the three nitrogen containing triazole compounds “P106” and “P110” of WO 2016/030229, such as in in vitro assays regarding flea membrane feeding (ingestion, blood feeding) activity against Ctenocephalides felis and/or contact activity against Rhipicephalus sanguineus and/or in in vivo assays in a rat-tick model and/or directly in/on dogs against ticks (e.g. Rhipicephalus sanguineus and/or Dermacentor variabilis). Furthermore, the compounds of formula (I), (Ia) or (Ib) as herein disclosed and/or claimed or a pharmaceutically acceptable salt thereof show advantageously an improved aqueous solubility, as compared to the structurally closest prior art, that is the three nitrogen containing triazole compounds “P106” and “P110” of WO 2016/030229, such as an improved aqueous solubility from DMSO stock solution dissolved in acetonitrile/water (1/1) solution compared to buffer using LC-UV. Moreover, the compounds of formula (I), (Ia) or (Ib) as herein disclosed and/or claimed or a pharmaceutically acceptable salt thereof show an advantageously higher degree of metabolic stability, as compared to the structurally closest prior art, that is the three nitrogen containing triazole compounds “P106” and “P110” of WO 2016/030229, such as an improved metabolic stability with rat liver microsomes in vitro and/or an improved metabolic stability with dog hepatocytes in vitro.

### DETAILED DESCRIPTION OF THE INVENTION

Generally, the present invention provides a compound of formula (I), (Ia) or (Ib) or a pharmaceutically acceptable salt thereof as herein disclosed and/or claimed as well as their corresponding pharmaceutical compositions, combinations and uses.

In a specific aspect, a compound of formula (I), (Ia) or (Ib) as herein disclosed and/or claimed is provided, wherein Q1 is Q1, Q2 or Q3, which independently are selected from the group consisting of:

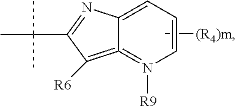

wherein R₄, R6, R9, m are as defined as herein disclosed and/or claimed, such as R6 is “hydrogen” or “F”, R9 is “CHF₂” or “CH₂—CHF₂” or “ethyl” or “CH₂-cyclopropyl”, m is 1 and R₄ is “CF₃” or “CHF₂” or “CF₂—CH₃”;

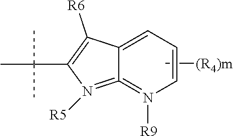

wherein R₄, R5, R6, m are as defined as herein disclosed and/or claimed, such as R5 is “hydrogen” or “methyl”, m is 1 and R₄ is “CF₂CF₃” or “CHF₂” or “CF₂—CH₃” and R6 is “hydrogen”;

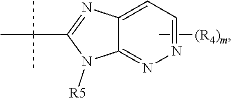

wherein R₄, R5, m are as defined as herein disclosed and/or claimed, such as R5 is “methyl”, m is 1 and R₄ is “CHF₂” or “CF₂-cyclopropyl” or “CF₂—CF₃” or “CF₂—CH₂—CH₃” or “CF₃” or “S—CF₃” or “S(O)₂—CF₃” or “O—CH₂—CF₃” or “CF₂—CH₃”;

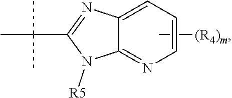

wherein R₄, R5, m are as defined as herein disclosed and/or claimed, such as R5 is “methyl”, m is 1 and R₄ is “CHF₂” or “CF₂—CF₃” or “CF₂-cyclopropyl” or “CF₂—CH₂—CH₃” or “S(O)₂—CF₃” or “CF₂—CH₃”;

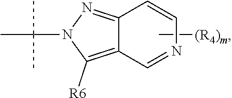

wherein R₄, R6, m are as defined as herein disclosed and/or claimed, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃” or “CF₂—CH₃”;

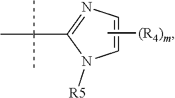

wherein R₄, R5, m are as defined as herein disclosed and/or claimed, such as R5 is “methyl”, m is 1 and R₄ is “C(O)NH—CH₂—CF₃” or “CF₂—CH₃”;
or a salt thereof.

In a specific aspect, a compound of formula (I), (Ia) or (Ib) as herein disclosed and/or claimed is provided, wherein Q is Q1, Q2 or Q3, which independently are selected from the group consisting of:

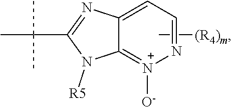

wherein R₄, R5, m are as defined as herein disclosed and/or claimed, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃” or “CF₂—CH₃”;

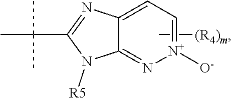

wherein R₄, R5, m are as defined as herein disclosed and/or claimed, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃” or “CF₂—CH₃”;

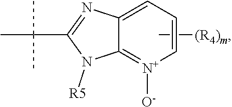

wherein R₄, R5, m are as defined as herein disclosed and/or claimed, such as R5 is “methyl”, m is 1 and R₄ is “CHF₂” or “CF₂—CF₃” or “CF₂-cyclopropyl” or “CF₂—CH₂—CH₃” or “S(O)₂—CF₃” or “CF₂—CH₃”;

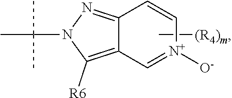

wherein R₄, R6, m are as defined as herein disclosed and/or claimed, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃” or “CF₂—CH₃”;
or a salt thereof.

In a specific aspect, a compound of formula (I), (Ia) or (Ib) as herein disclosed and/or claimed is provided, wherein optionally the at least one nitrogen atom being an N-oxide comprises the one of the central pyridine moiety selected from the group consisting of formula (II) or formula (IIa) or formula (IIb):

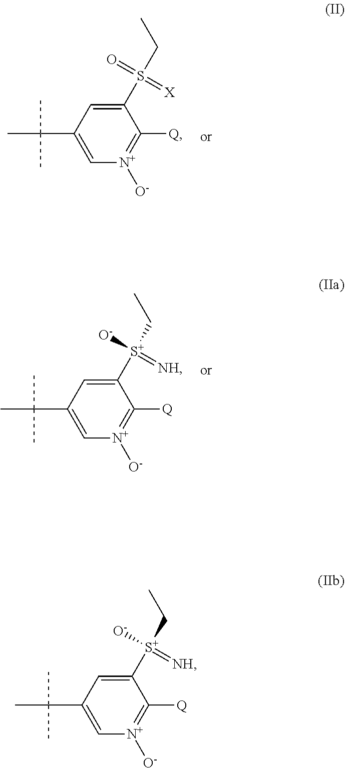

or a salt thereof,
wherein X, if present, and Q are as defined as herein disclosed and/or claimed and formulae (II), (IIa) and (IIb) are only part of the corresponding formulae (I), (Ia) and (Ib), respectively.

### Definitions

Unless defined otherwise, all technical and scientific terms used herein have the same meaning as is commonly understood by one of skill in the art to which this invention belongs at the time of filing. Terms not specifically defined herein should be given the meanings that would be given to them by one of skill in the art in light of the disclosure and the context. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular. Herein, the use of “or” means “and/or” unless stated otherwise. Furthermore, the use of the term “including”, as well as other forms such as “includes” and “included” is not limiting. As used in the specification, however, unless specified to the contrary, the following terms have the meaning indicated and the following conventions are adhered to.

In the groups, radicals, or moieties defined herein, the number of carbon atoms is often specified preceding the group, for example, C₁₋₆-alkyl means an alkyl group or radical having 1 to 6 carbon atoms. In general, in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last-named subgroup is the radical attachment point, for example, the substituent “aryl-C₁₋₃-alkyl” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk may be used in sub-formulas to indicate the bond which is connected to the core molecule as defined.

The numeration of the atoms of a substituent starts with the atom which is closest to the core or to the group to which the substituent is attached. For example, the term “3-carboxypropyl-group” represents the following substituent:

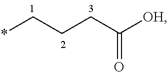 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

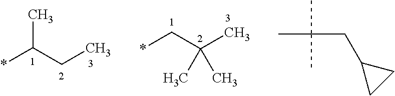

The asterisk or

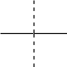

may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g., “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc.) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g., by separation of corresponding mixtures, by using stereochemically pure starting materials and/or by stereoselective synthesis. It is known in the art how to prepare optically active forms, such as by resolution of racemic forms or by synthesis, e.g., starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term “halogen” denotes fluorine, chlorine, bromine and iodine.

The term “C₁₋ₙalkyl”, wherein n is an integer selected from 2, 3, 4, 5 or 6, either alone or in combination with another radical, denotes an acyclic, saturated, branched or linear hydrocarbon radical with 1 to n C atoms. For example the term C₁₋₅alkyl embraces the radicals H₃C, H₃CCH₂, H₃CCH₂CH₂, H₃CCH(CH₃), H₃CCH₂CH₂CH₂, H₃CCH₂CH(CH₃), H₃CCH(CH₃)CH₂, H₃CC(CH₃)₂, H₃CCH₂CH₂CH₂CH₂, H₃CCH₂CH₂CH(CH₃), H₃CCH₂CH(CH₃)CH₂, H₃CCH(CH₃)CH₂CH₂, H₃CCH₂C(CH₃)₂, H₃CC(CH₃)₂CH₂, H₃CCH(CH₃)CH(CH₃) and H₃CCH₂CH(CH₂CH₃).

The term “C₁₋ₙalkylene”, wherein n is an integer selected from 2, 3, 4, 5 or 6, either alone or in combination with another radical, denotes an acyclic, saturated, branched or linear chain divalent alkyl radical containing from 1 to n carbon atoms. For example, the term C₁₋₄alkylene includes CH₂, CH₂CH₂, CH(CH₃), CH₂CH₂CH₂, C(CH₃)₂, CH(CH₂CH₃), CH(CH₃)CH₂, CH₂CH(CH₃), CH₂CH₂CH₂CH₂, CH₂CH₂CH(CH₃), CH(CH₃)CH₂CH₂, CH₂CH(CH₃)CH₂, CH₂C(CH₃)₂, C(CH₃)₂CH₂, CH(CH₃)CH(CH₃), CH₂CH(CH₂CH₃), CH(CH₂CH₃)CH₂, CH(CH₂CH₂CH₃), CH(CH(CH₃))₂ and C(CH₃)(CH₂CH₃).

The term “C₂₋ₘ-alkenyl” is used for a group “C₂₋ₘ-alkyl”, wherein m is an integer selected from 3, 4, 5 or 6, if at least two carbon atoms of said group are bonded to each other by a double bond.

The term “C₂₋ₘ-alkenylene” is used for a group “C₂₋ₘ-alkylene”, wherein m is an integer selected from 3, 4, 5 or 6, if at least two carbon atoms of said group are bonded to each other by a double bond.

The term “C₂₋ₘ-alkynyl” is used for a group “C₂₋ₘ-alkyl”, wherein m is an integer selected from 3, 4, 5 or 6, if at least two carbon atoms of said group are bonded to each other by a triple bond.

The term “C₂₋ₘ-alkynylene” is used for a group “C₂₋ₘ-alkylene”, wherein m is an integer selected from 3, 4, 5 or 6, if at least two of those carbon atoms of said group are bonded to each other by a triple bond.

The term “C₃₋ₖcycloalkyl”, wherein k is an integer selected from 4, 5, 6, 7 or 8, either alone or in combination with another radical, denotes a cyclic, saturated, unbranched hydrocarbon radical with 3 to k C atoms. For example, the term C₃₋₇cycloalkyl includes cyclopropyl, cyclobutyl, cyclopentyl, cyclohexyl and cycloheptyl.

The term “C₃₋ₖcycloalkenyl”, wherein k is an integer selected from 4, 5, 6, 7 or 8, either alone or in combination with another radical, denotes a cyclic, unsaturated, but non-aromatic, unbranched hydrocarbon radical with 3 to k C atoms, at least two of which are bonded to each other by a double bond. For example, the term C₃₇cycloalkenyl includes cyclopropenyl, cyclobutenyl, cyclopentenyl, cyclopentadienyl, cyclohexenyl, cyclohexadienyl, cycloheptenyl, cycloheptadienyl and cycloheptatrienyl.

The term “halo” added to an “alkyl”, “alkylene”, “alkenyl”, “alkenylene”, “alkynyl”, “alkynylene”, “cycloalkyl”, “cycloalkenyl” or “alkoxy” group defines an alkyl, alkylene, alkenyl, alkenylene, alkynyl, alkynylene, cycloalkyl, cycloalkenyl or alkoxy group, wherein one or more hydrogen atoms are replaced by a halogen atom selected from among fluorine, chlorine, bromine or iodine. For example C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “fluoroalkyl” as used herein refers to an alkyl in which one or more of the hydrogen atoms is replaced with fluorine atoms, for example difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “alkoxy” refers to an alkyl-O—, wherein alkyl is as defined above. Similarly, the terms “alkenyloxy”, “alkynyloxy”, “haloalkoxy”, “haloalkenyloxy”, “haloalkynyloxy”, “cycloalkoxy”, “cycloalkenyloxy”, “halocycloalkoxy”, and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O—, and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined herein. Examples of C₁-C₆-alkoxy include, but are not limited to, methoxy, ethoxy, OCH₂—C₂H₅, OCH(CH₃)₂, n-butoxy, OCH(CH₃)—C₂H5, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

The term “carbocyclyl” or “carbocycle”, either alone or in combination with another radical, means a mono, bi or tricyclic ring structure consisting of 3 to 14 carbon atoms. The term “carbocyclyl” or “carbocycle” refers to fully saturated, partially saturated or aromatic ring systems. The term “carbocyclyl” or “carbocycle” encompasses fused, bridged and spirocyclic systems. Examples include:

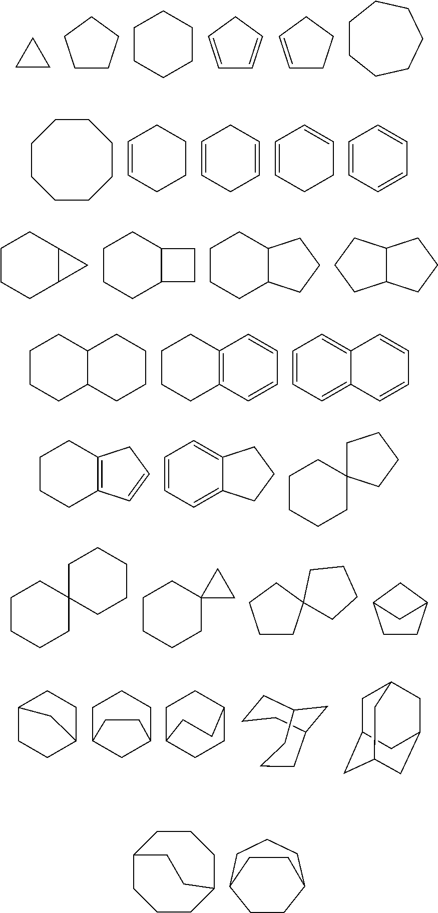

The term “aryl”, either alone or in combination with another radical, denotes a carbocyclic aromatic monocyclic group containing 6 carbon atoms, which is optionally further fused to a second five- or six-membered, carbocyclic group which is aromatic, fully saturated or partially saturated. The term “aryl” includes, but is not limited to, phenyl, indanyl, indenyl, naphthyl, anthracenyl, phenanthrenyl, tetrahydronaphthyl and dihydronaphthyl.

The term “aralkyl” refers to an aryl group that is bonded to the parent compound through a diradical alkylene bridge, (—CH₂—)ₙ, where n is 1-6 and where “aryl” is as defined herein.

The term “heterocyclyl” or “heterocycle” means a saturated or unsaturated mono- or polycyclic ring system optionally comprising aromatic rings, containing one or more heteroatoms selected from N, O, S, S(O) or S(O)₂ consisting of 3 to 14 ring atoms, wherein none of the heteroatoms is part of the aromatic ring. The term “heterocyclyl” or “heterocycle” is intended to include all possible isomeric forms. Thus, the term “heterocyclyl” or “heterocycle” includes the following exemplary structures (not depicted as radicals as each form is optionally attached through a covalent bond to any atom so long as appropriate valences are maintained):

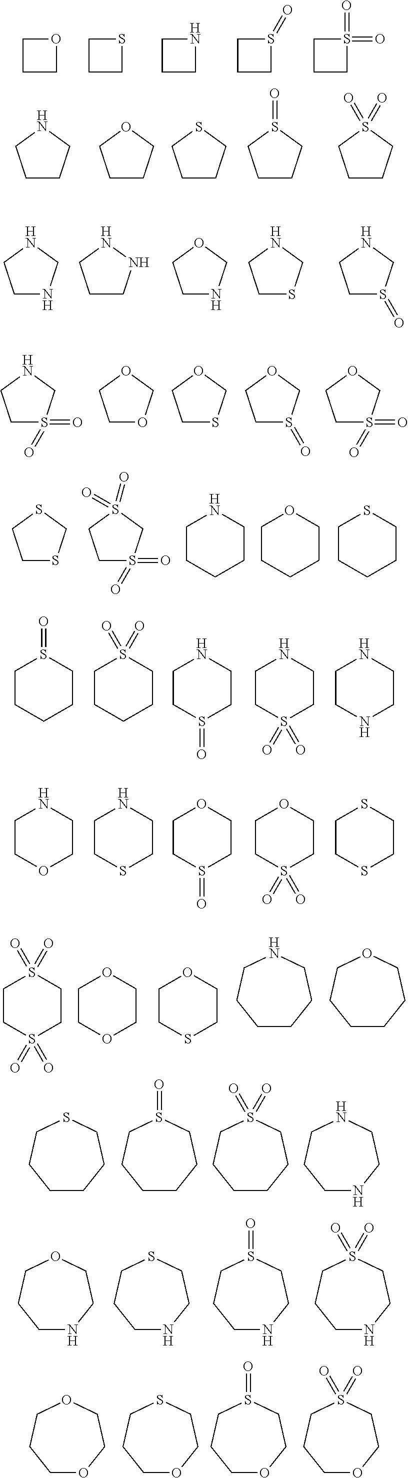

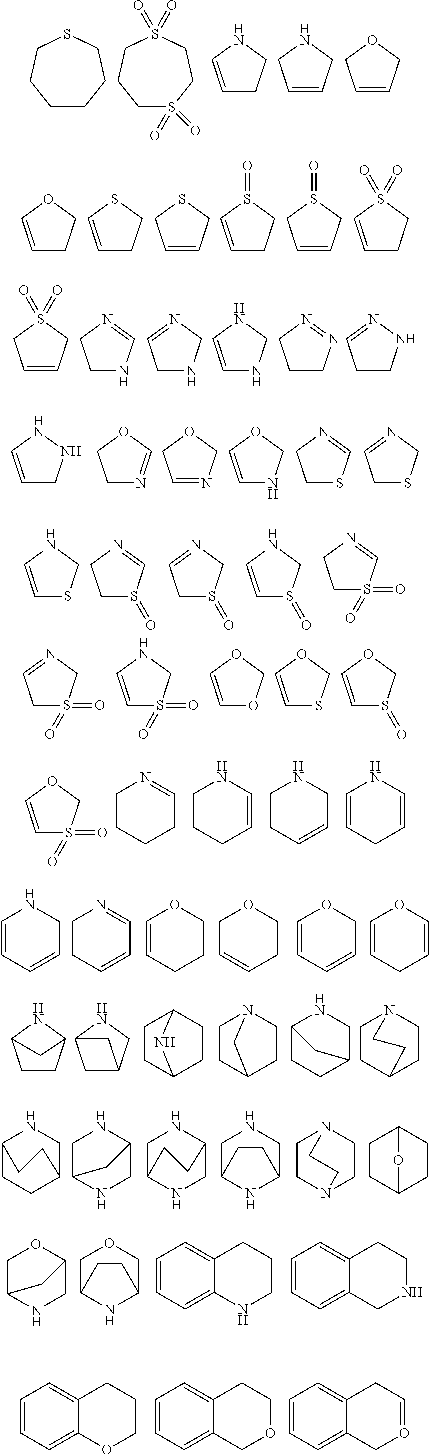

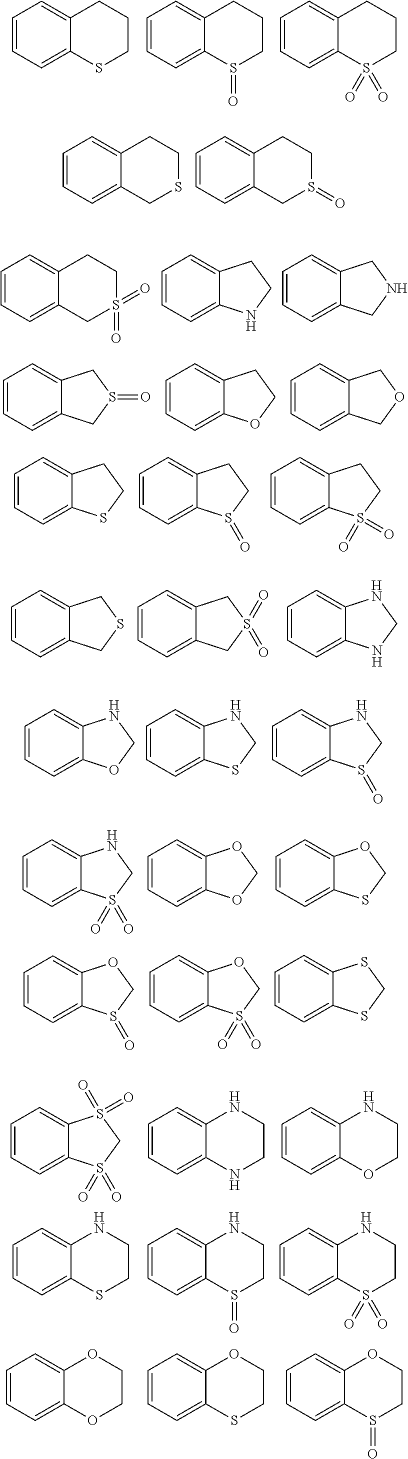

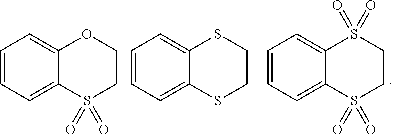

The term “heteroaryl” means a mono- or polycyclic ring system, comprising at least one aromatic ring, containing one or more heteroatoms selected from N, O, S, S(O) or S(O)₂, consisting of 5 to 14 ring atoms, wherein at least one of the heteroatoms is part of an aromatic ring. The term “heteroaryl” is intended to include all the possible isomeric forms. Thus, the term “heteroaryl” includes the following exemplary structures (not depicted as radicals as each form is optionally attached through a covalent bond to any atom so long as appropriate valences are maintained):

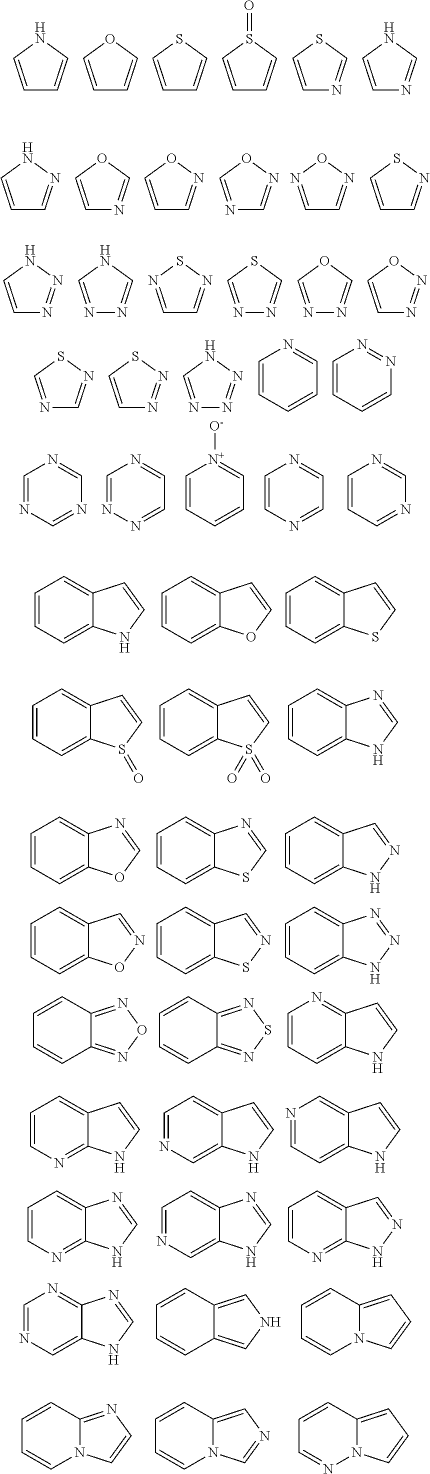

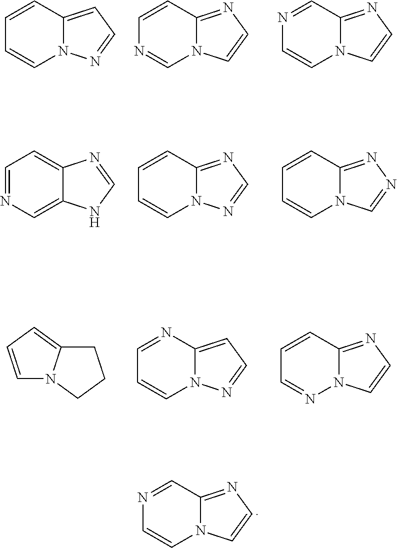

Many of the terms given herein may be used repeatedly in the definition of a formula or group and in each case have one of the meanings given herein, independently of one another.

The term “bicyclic ring systems” means groups consisting of 2 joined cyclic substructures including spirocyclic, fused, and bridged ring systems.

The term “tricyclic ring systems” means groups consisting of 3 joined cyclic substructures including spirocyclic, fused, and bridged ring systems.

As used herein, the term “prevention” in connection with “parasitic infections and/or infestations in/on animals” means prophylactical treatment of a given animal. For instance, the action of stopping endoparasitic infections or ectoparasitic infestations from establishing, usually for a defined post-treatment time interval, such as the protective period, i.e. the time period, usually expressed in days or weeks after the treatment, that a product will kill a newly internalized endoparasite, thereby preventing endoparasitic (re-)infection from developing in the animal, or prevent re-infestation of the animal by the ectoparasite (sometimes also referred to as the prophylactic period or the persistent efficacy period), is a non-limiting example of such a prevention.

As used herein, the term “control” in connection with “parasitic infections and/or infestations in animals” means that the parasitic infection and/or infestation is ameliorated or improved, sustainedly reduced in incidence and/or prevented from worsening as regards the animal.

### Methods of Treatment

The compositions of the invention are intended to be administered to an animal including, but not limited to, mammals, birds and fish. Examples of mammals include but are not limited to humans, cattle, sheep, goats, llamas, alpacas, pigs, horses, donkeys, dogs, cats and other livestock or domestic mammals. Examples of birds include turkeys, chickens, ostriches and other livestock or domestic birds. In one embodiment, the invention provides a use of the compound to protect companion animals such as dogs and cats from endoparasites. In another embodiment, the compound of the invention may be used to protect equine animals from parasites. The compound may also be used to protect livestock animals.

The present invention is directed to compounds of formulae (I), (Ia) or (Ib), which are useful in the treatment, prevention and/or control of parasitic infections and/or infestations in/on animals, preferably of ectoparasitic infestations on animals, more preferably of infestations of fleas and/or ticks on animals.

Accordingly, the present invention relates to a compound of formula (I), (Ia) or (Ib) for use as a medicament, including, but not limited to, for use as an antiparasitic medicament.

Furthermore, the present invention relates to the use of a compound of formula (I), (Ta) or (Tb) for the treatment, prevention and/or control of parasitic infections and/or infestations in/on animals, preferably of ectoparasitic infestations on animals, more preferably of infestations of fleas and/or ticks on animals.

In a further aspect the present invention relates to a compound of formula (I), (Ia) or (Tb) for use in the treatment, prevention and/or control of parasitic infections and/or infestations in/on animals, preferably of ectoparasitic infestations on animals, more preferably of infestations of fleas and/or ticks on animals.

In a further aspect the present invention relates to the use of a compound of formula (I), (Ta) or (Tb) for the preparation of a medicament for the treatment, prevention and/or control of parasitic infections and/or infestations in/on animals, preferably of ectoparasitic infestations on animals, more preferably of infestations of fleas and/or ticks on animals.

In a further aspect of the present invention the present invention relates to methods for the treatment, prevention and/or control of parasitic infections and/or infestations in/on animals, preferably of ectoparasitic infestations on animals, more preferably of infestations of fleas and/or ticks on animals, which methods comprise the administration of an effective amount of a compound of formula (I), (Ia) or (Tb) to an animal/animal patient in need thereof.

The compounds of the present invention are highly effective for the treatment, prevention and/or control of external and/or internal parasites in animals, mammals, fish and birds, and in particular, cats, dogs, horses, chicken, pigs, sheep and cattle, but also humans with the aim of substantially ridding these hosts of ectoparasites and/or endoparasites. Mammals which can be treated, include but are not limited to, humans, cats, dogs, cattle, chicken, cows, bison, deer, goats, horses, llamas, camels, pigs, sheep and yaks. In one embodiment of the present invention, the mammals treated are humans, cats or dogs.

The dose range of the compounds of formulae (I), (Ia) and/or (Ib) applicable per day is usually from 0.001 mg to 1,000 mg for animals.

The actual pharmaceutically effective amount or therapeutic dosage will usually depend on factors known by those skilled in the art such as age and weight of the animal patient, route of administration and severity of disease. In any case the compounds of the invention will be administered at dosages and in a manner which allows a pharmaceutically effective amount to be delivered based upon the animal patient's unique condition.

The agricultural and horticultural insecticidal and acaricidal agent comprising the compounds of formulae (I), (Ia) and/or (Tb) of the present invention or a salt thereof as an active ingredient has a remarkable control effect on pests which damage lowland crops, field crops, fruit trees, vegetables, other crops, ornamental flowering plants, etc. The desired effect can be obtained when the agricultural and horticultural insecticidal and acaricidal agent is applied to nursery facilities for seedlings, paddy fields, fields, fruit trees, vegetables, other crops, ornamental flowering plants, etc. and their seeds, paddy water, foliage, cultivation media such as soil, or the like around the expected time of pest infestation, i.e., before the infestation or upon the confirmation of the infestation. In particularly preferable embodiments, the application of the agricultural and horticultural insecticidal and acaricidal agent utilizes so-called penetration and translocation. That is, nursery soil, soil in transplanting holes, plant foot, irrigation water, cultivation water in hydroponics, or the like is treated with the agricultural and horticultural insecticidal and acaricidal agent to allow crops, ornamental flowering plants, etc. to absorb the compound of the present invention through the roots via soil or otherwise.

Examples of useful plants to which the agricultural and horticultural insecticidal and acaricidal agent of the present invention can be applied include, but are not particularly limited to, cereals (e.g., rice, barley, wheat, rye, oats, corn, etc.), legumes (e.g., soybeans, azuki beans, broad beans, green peas, kidney beans, peanuts, etc.), fruit trees and fruits (e.g., apples, citrus fruits, pears, grapes, peaches, plums, cherries, walnuts, chestnuts, almonds, bananas, etc.), leaf and fruit vegetables (e.g., cabbages, tomatoes, spinach, broccoli, lettuce, onions, green onions (chives and Welsh onions), green peppers, eggplants, strawberries, pepper crops, okra, Chinese chives, etc.), root vegetables (e.g., carrots, potatoes, sweet potatoes, taros, Japanese radishes, turnips, lotus roots, burdock roots, garlic, Chinese scallions, etc.), crops for processing (e.g., cotton, hemp, beet, hops, sugarcane, sugar beet, olives, rubber, coffee, tobacco, tea, etc.), gourds (e.g., Japanese pumpkins, cucumbers, watermelons, oriental sweet melons, melons, etc.), pasture grass (e.g., orchardgrass, sorghum, timothy, clover, alfalfa, etc.), lawn grass (e.g., Korean lawn grass, bent grass, etc.), spice and aromatic crops and ornamental crops (e.g., lavender, rosemary, thyme, parsley, pepper, ginger, etc.), ornamental flowering plants (e.g., chrysanthemum, rose, carnation, orchid, tulip, lily, etc.), garden trees (e.g., ginkgo trees, cherry trees, Japanese aucuba, etc.) and forest trees (e.g., Abies sachalinensis, Picea jezoensis, pine, yellow cedar, Japanese cedar, hinoki cypress, eucalyptus, etc.).

The above-mentioned “plants” also include plants provided with herbicide tolerance by a classical breeding technique or a gene recombination technique. Examples of such herbicide tolerance include tolerance to HPPD inhibitors, such as isoxaflutole; ALS inhibitors, such as imazethapyr and thifensulfuron-methyl; EPSP synthase inhibitors, such as glyphosate; glutamine synthetase inhibitors, such as glufosinate; acetyl-CoA carboxylase inhibitors, such as sethoxydim; or other herbicides, such as bromoxynil, dicamba and 2,4-D.

Examples of the plants provided with herbicide tolerance by a classical breeding technique include varieties of rapeseed, wheat, sunflower and rice tolerant to the imidazolinone family of ALS-inhibiting herbicides such as imazethapyr, and such plants are sold under the trade name of Clearfield (registered trademark). Also included is a variety of soybean provided with tolerance to the sulfonyl urea family of ALS-inhibiting herbicides such as thifensulfuron-methyl by a classical breeding technique, and this is sold under the trade name of STS soybean. Also included are plants provided with tolerance to acetyl-CoA carboxylase inhibitors such as trione oxime herbicides and aryloxy phenoxy propionic acid herbicides by a classical breeding technique, for example, SR corn and the like.

Plants provided with tolerance to acetyl-CoA carboxylase inhibitors are described in Proc. Natl. Acad. Sci. USA, 87, 7175-7179 (1990), and the like. Further, acetyl-CoA carboxylase mutants resistant to acetyl-CoA carboxylase inhibitors are reported in Weed Science, 53, 728-746 (2005), and the like, and by introducing the gene of such an acetyl-CoA carboxylase mutant into plants by a gene recombination technique, or introducing a resistance-conferring mutation into acetyl-CoA carboxylase of plants, plants tolerant to acetyl-CoA carboxylase inhibitors can be engineered. Alternatively, by introducing a nucleic acid causing base substitution mutation into plant cells (a typical example of this technique is chimeraplasty technique (Gura T. 1999. Repairing the Genome's Spelling Mistakes. Science 285: 316-318)) to allow site-specific substitution mutation in the amino acids encoded by an acetyl-CoA carboxylase gene, an ALS gene or the like of plants, plants tolerant to acetyl-CoA carboxylase inhibitors, ALS inhibitors or the like can be engineered. The agricultural and horticultural insecticidal and acaricidal agent of the present invention can be applied to these plants as well.

Further, exemplary toxins expressed in genetically modified plants include insecticidal proteins of Bacillus cereus or Bacillus popilliae; Bacillus thuringiensis δ-endotoxins, such as Cry1Ab, Cry1Ac, Cry1F, Cry1Fa2, Cry2Ab, Cry3A, Cry3Bb1 and Cry9C, and other insecticidal proteins, such as VIP1, VIP2, VIP3 and VIP3A; nematode insecticidal proteins; toxins produced by animals, such as scorpion toxins, spider toxins, bee toxins and insect-specific neurotoxins; toxins of filamentous fungi; plant lectins; agglutinin; protease inhibitors, such as trypsin inhibitors, serine protease inhibitors, patatin, cystatin and papain inhibitors; ribosome inactivating proteins (RIP), such as ricin, maize RIP, abrin, luffin, saporin and bryodin; steroid metabolizing enzymes, such as 3-hydroxy steroid oxidase, ecdysteroid-UDP-glucosyltransferase and cholesterol oxidase; ecdysone inhibitors; HMG-CoA reductase; ion channel inhibitors, such as sodium channel inhibitors and calcium channel inhibitors; juvenile hormone esterase; diuretic hormone receptors; stilbene synthase; bibenzyl synthase; chitinase; and glucanase.

Due to the toxins contained in such genetically modified plants, the plants exhibit resistance to pests, in particular, Coleopteran insect pests, Hemipteran insect pests, Dipteran insect pests, Lepidopteran insect pests and nematodes. The above-described technologies and the agricultural and horticultural insecticidal and acaricidal agent of the present invention can be used in combination or used systematically.

In order to control target pests, the agricultural and horticultural insecticidal and acaricidal agent of the present invention, with or without appropriate dilution or suspension in water etc., is applied to plants potentially infested with the target insect pests or nematodes in an amount effective for the control of the insect pests or nematodes. For example, in order to control insect pests and nematodes that may damage crop plants such as fruit trees, cereals and vegetables, foliar application and seed treatment such as dipping, dust coating and calcium peroxide coating can be performed. Further, treatment of soil or the like may also be performed to allow plants to absorb agrochemicals through their roots. Examples of such treatment include whole soil incorporation, planting row treatment, bed soil incorporation, plug seedling treatment, planting hole treatment, plant foot treatment, top-dressing, treatment of nursery boxes for paddy rice, and submerged application. In addition, application to culture media in hydroponics, smoking treatment, trunk injection and the like can also be performed. Further, the agricultural and horticultural insecticidal and acaricidal agent of the present invention, with or without appropriate dilution or suspension in water etc., can be applied to sites potentially infested with pests in an amount effective for the control of the pests. For example, it can be directly applied to stored grain pests, house pests, sanitary pests, forest pests, etc., and also be used for coating of residential building materials, for smoking treatment, or as a bait formulation.

Exemplary methods of seed treatment include dipping of seeds in a diluted or undiluted fluid of a liquid or solid formulation for the permeation of agrochemicals into the seeds; mixing or dust coating of seeds with a solid or liquid formulation for the adherence of the formulation onto the surfaces of the seeds; coating of seeds with a mixture of an agrochemical and an adhesive carrier such as resins and polymers; and application of a solid or liquid formulation to the vicinity of seeds at the same time as seeding.

The term “seed” in the above-mentioned seed treatment refers to a plant body which is in the early stages of cultivation and used for plant propagation. The examples include, in addition to a so-called seed, a plant body for vegetative propagation, such as a bulb, a tuber, a seed potato, a bulbil, a propagule, a discoid stem and a stem used for cuttage.

The term “soil” or “cultivation medium” in the method of the present invention for using an agricultural and horticultural insecticide refers to a support medium for crop cultivation, in particular a support medium which allows crop plants to spread their roots therein, and the materials are not particularly limited as long as they allow plants to grow. Examples of the support medium include what is called soils, seedling mats and water, and specific examples of the materials include sand, pumice, vermiculite, diatomite, agar, gelatinous substances, high-molecular-weight substances, rock wool, glass wool, wood chip and bark.

Exemplary methods of the application to crop foliage or to stored grain pests, house pests, sanitary pests, forest pests, etc. include application of a liquid formulation, such as an emulsifiable concentrate and a flowable, or a solid formulation, such as a wettable powder and a water-dispersible granule, after appropriate dilution in water; dust application; and smoking.

Exemplary methods of soil application include application of a water-diluted or undiluted liquid formulation to the foot of plants, nursery beds for seedlings, or the like; application of a granule to the foot of plants, nursery beds for seedlings, or the like; application of a dust, a wettable powder, a water-dispersible granule, a granule or the like onto soil and subsequent incorporation of the formulation into the whole soil before seeding or transplanting; and application of a dust, a wettable powder, a water-dispersible granule, a granule or the like to planting holes, planting rows or the like before seeding or planting.

To nursery boxes for paddy rice, for example, a dust, a water-dispersible granule, a granule or the like can be applied, although the suitable formulation may vary depending on the application timing, in other words, depending on the cultivation stage such as seeding time, greening period and planting time. A formulation such as a dust, a water-dispersible granule and a granule may be mixed with nursery soil. For example, such a formulation is incorporated into bed soil, covering soil or the whole soil. Simply, nursery soil and such a formulation may be alternately layered.

In the application to paddy fields, a solid formulation, such as a jumbo, a pack, a granule and a water-dispersible granule, or a liquid formulation, such as a flowable and an emulsifiable concentrate, is applied usually to flooded paddy fields. In a rice planting period, a suitable formulation, as it is or after mixed with a fertilizer, may be applied onto soil or injected into soil. In addition, an emulsifiable concentrate, a flowable or the like may be applied to the source of water supply for paddy fields, such as a water inlet and an irrigation device. In this case, treatment can be accomplished with the supply of water and thus achieved in a labor-saving manner.

In the case of field crops, their seeds, cultivation media in the vicinity of their plants, or the like may be treated in the period of seeding to seedling culture. In the case of plants of which the seeds are directly sown in the field, in addition to direct seed treatment, plant foot treatment during cultivation is preferable. Specifically, the treatment can be performed by, for example, applying a granule onto soil, or drenching soil with a formulation in a water-diluted or undiluted liquid form. Another preferable treatment is incorporation of a granule into cultivation media before seeding.

In the case of culture plants to be transplanted, preferable examples of the treatment in the period of seeding to seedling culture include, in addition to direct seed treatment, drench treatment of nursery beds for seedlings with a formulation in a liquid form; and granule application to nursery beds for seedlings. Also included are treatment of planting holes with a granule; and incorporation of a granule into cultivation media in the vicinity of planting points at the time of fix planting.

The amount of the active ingredient compound in the agricultural and horticultural insecticidal and acaricidal agent of the present invention can be adjusted as needed, and basically, the amount of the active ingredient compound is appropriately selected from the range of 0.01 to 90 parts by weight in 100 parts by weight of the agricultural and horticultural insecticide. For example, in the case where the agricultural and horticultural insecticide is a dust, a granule, an emulsifiable concentrate or a wettable powder, it is suitable that the amount of the active ingredient compound is 0.01 to 50 parts by weight (0.01 to 50% by weight relative to the total weight of the agricultural and horticultural insecticidal and acaricidal agent).

The application rate of the agricultural and horticultural insecticidal and acaricidal agent of the present invention may vary with various factors, for example, the purpose, the target pest, the growing conditions of crops, the tendency of pest infestation, the weather, the environmental conditions, the dosage form, the application method, the application site, the application timing, etc., but basically, the application rate of the active ingredient compound is appropriately selected from the range of 0.001 g to 10 kg, and preferably 0.01 g to 1 kg per 10 ares depending on the purpose.

### Veterinary Parasites

In one embodiment, the compounds and pharmaceutical compositions of the invention may be used for treating, controlling and/or preventing an endoparasitic infection of the following parasite genera: Anoplocephala, Ancylostoma, Necator, Ascaris, Brugia, Bunostomum, Capillaria, Chabertia, Cooperia, Cyathostomum, Cylicocyclus, Cylicodontophorus, Cylicostephanus, Craterostomum, Dictyocaulus, Dipetalonema, Dipylidium, Dirofilaria, Dracunculus, Echinococcus, Enterobius, Fasciola, Filaroides, Habronema, Haemonchus, Metastrongylus, Moniezia, Nematodirus, Nippostrongylus, Oesophagostomum, Onchocerca, Ostertagia, Oxyuris, Parascaris, Schistosoma, Strongylus, Taenia, Toxocara, Strongyloides, Toxascaris, Trichinella, Trichuris, Trichostrongylus, Triodontophorus, Uncinaria, Wuchereria, and combinations thereof.

In one embodiment, the compounds and compositions of the invention may be used to treat, control and/or prevent an infection by filarial parasites such as Dirofilaria immitis. In another embodiment the compounds and compositions of the invention are used to treat, control and/or prevent an infection by Dirofilaria repens or Dirofilaria hongkongensis. In another embodiment, the compounds of formulae (I), (Ia) and/or (Ib) may be used to treat, control and/or prevent a parasitic infection by a parasite selected from Haemonchus contortus, Ostertagia circumcincta, Trichostrongylus axei, Trichostrongylus colubriformis, Cooperia curticei, Nematodirus battus and combinations thereof.

In one embodiment for the treatment, prevention and/or control of/against ectoparasites, the ectoparasite is one or more insect or arachnid including those of the genera Ctenocephalides, Rhipicephalus, Dermacentor, Ixodes, Boophilus, Amblyomma, Haemaphysalis, Hyalomma, Sarcoptes, Psoroptes, Otodectes, Chorioptes, Hypoderma, Gasterophilus, Lucilia, Dermatobia, Cochliomyia, Chrysomya, Damalinia, Linognathus, Haematopinus, Solenopotes, Trichodectes, and Felicola.

In another embodiment for the treatment, prevention and/or control of/against ectoparasites, the ectoparasite is from the genera Ctenocephalides, Rhipicephalus, Dermacentor, Ixodes and/or Boophilus. The ectoparasites treated include but are not limited to fleas, ticks, mites, mosquitoes, flies, lice, blowfly and combinations thereof. Specific examples include, but are not limited to, cat and dog fleas (Ctenocephalides felis, Ctenocephalides canis, Ctenocephalides sp. and the like), ticks (Rhipicephalus sp., such as Rhipicephalus sanguineus (brown dog tick), Ixodes sp., such as Ixodes scapularis (black-legged tick), Ixodes ricinus, and Ixodes hexagonus, Dermacentor sp., such as Dermacentor variabilis (American dog tick), and Dermacentor reticulatus, Amblyomma sp., such as Amblyomma americanum (lone star tick), Haemaphysalis sp., such as Haemaphysalis longicornis (longhorned tick), and the like), and mites (Demodex sp., such as Demodex canis, Sarcoptes sp., such as Sarcoptes scabiei var. canis, Otodectes sp., Cheyletiella sp., and the like), lice (Trichodectes sp., Felicola sp., Linognathus sp., and the like), mosquitoes (Aedes sp., Culex sp., Anopheles sp., and the like) and flies (Haematobia sp. including Haematobia irritans, Musca sp., Stomoxys sp. including Stomoxys calcitrans, Dermatobia sp., Cochliomyia sp., and the like).

Additional examples of ectoparasites include, but are not limited to, the tick genus Boophilus, especially those of the species microplus (cattle tick), decoloratus and annulatus; myiases such as Dermatobia hominis (known as Berne in Brazil) and Cochliomyia hominivorax (greenbottle); sheep myiases such as Lucilia sericata, Lucilia cuprina (known as blowfly strike in Australia, New Zealand and South Africa) and Gasterophilus in horses. Flies proper, namely those whose adult constitutes the parasite, such as Haematobia irritans (horn fly) and Stomoxys calcitrans (stable fly); lice such as Linognathus vituli, Bovicola spp. (e.g., Bovicola ovis, Bovicola bovis), etc.; and mites such as Sarcoptes scabiei and Psoroptes ovis. The herein disclosed list is not exhaustive and other ectoparasites are well known in the art to be harmful to animals and humans. These include, for example migrating dipteran larvae.

In each aspect of the invention, the compounds and pharmaceutical compositions of the invention can be applied against a single pest or combinations thereof.

### Agricultural and Horticultural Pests and Nematodes

The agricultural and horticultural insecticidal and acaricidal agent comprising the compounds of formulae (I), (Ia) and/or (Ib) of the present invention or a salt thereof as an active ingredient is suitable for controlling a variety of pests which may damage paddy rice, fruit trees, vegetables, other crops and ornamental flowering plants. The target pests are, for example, agricultural and forest pests, horticultural pests, stored grain pests, sanitary pests, other pests such as nematodes, or mites, etc.

Examples of the above pests or nematodes include the following.

Examples of the species of the order Lepidoptera include Parasa consocia, Anomis mesogona, Papilio xuthus, Matsumuraeses azukivora, Ostrinia scapulalis, Spodoptera exempta, Hyphantria cunea, Ostrinia furnacalis, Pseudaletia separata, Tinea translucens, Bactra furfurana, Parnara guttata, Marasmia exigua, Parnara guttata, Sesamia inferens, Brachmia triannulella, Monema flavescens, Trichoplusia ni, Pleuroptya ruralis, Cystidia couaggaria, Lampides boeticus, Cephonodes hylas, Helicoverpa armigera, Phalerodonta manleyi, Eumeta japonica, Pieris brassicae, Malacosoma neustria testacea, Stathmopoda masinissa, Cuphodes diospyrosella, Archips xylosteanus, Agrotis segetum, Tetramoera schistaceana, Papilio machaon hippocrates, Endoclyta sinensis, Lyonetia prunifoliella, Phyllonorycter ringoneella, Cydia kurokoi, Eucoenogenes aestuosa, Lobesia botrana, Latoia sinica, Euzophera batangensis, Phalonidia mesotypa, Spilosoma imparilis, Glyphodes pyloalis, Olethreutes mori, Tineola bisselliella, Endoclyta excrescens, Nemapogon granellus, Synanthedon hector, Cydia pomonella, Plutella xylostella, Cnaphalocrocis medinalis, Sesamia calamistis, Scirpophaga incertulas, Pediasia teterrellus, Phthorimaea operculella, Stauropus fagi persimilis, Etiella zinckenella, Spodoptera exigua, Palpifer sexnotata, Spodoptera mauritia, Scirpophaga innotata, Xestia c-nigrum, Spodoptera depravata, Ephestia kuehniella, Angerona prunaria, Clostera anastomosis, Pseudoplusia includens, Matsumuraeses falcana, Helicoverpa assulta, Autographa nigrisigna, Agrotis ipsilon, Euproctis pseudoconspersa, Adoxophyes orana, Caloptilia theivora, Homona magnanima, Ephestia elutella, Eumeta minuscula, Clostera anachoreta, Heliothis maritima, Sparganothis pilleriana, Busseola fusca, Euproctis subflava, Biston robustum, Heliothis zea, Aedia leucomelas, Narosoideus flavidorsalis, Viminia rumicis, Bucculatrix pyrivorella, Grapholita molesta, Spulerina astaurota, Ectomyelois pyrivorella, Chilo suppressalis, Acrolepiopsis sapporensis, Plodia interpunctella, Hellula undalis, Sitotroga cerealella, Spodoptera litura, a species of the family Tortricidae (Eucosma aporema), Acleris comariana, Scopelodes contractus, Orgyia thyellina, Spodoptera frugiperda, Ostrinia zaguliaevi, Naranga aenescens, Andraca bipunctata, Paranthrene regalis, Acosmeryx castanea, Phyllocnistis toparcha, Endopiza viteana, Eupoecillia ambiguella, Anticarsia gemmatalis, Cnephasia cinereipalpana, Lymantria dispar, Dendrolimus spectabilis, Leguminivora glycinivorella, Maruca testulalis, Matsumuraeses phaseoli, Caloptilia soyella, Phyllocnistis citrella, Omiodes indicata, Archips fuscocupreanus, Acanthoplusia agnata, Bambalina sp., Carposina niponensis, Conogethes punctiferalis, Synanthedon sp., Lyonetia clerkella, Papilio helenus, Colias erate poliographus, Phalera flavescens, the species of the family Pieridae such as Pieris rapae crucivora and Pieris rapae, Euproctis similis, Acrolepiopsis suzukiella, Ostrinia nubilalis, Mamestra brassicae, Ascotis selenaria, Phtheochroides clandestina, Hoshinoa adumbratana, Odonestis pruni japonensis, Triaena intermedia, Adoxophyes orana fasciata, Grapholita inopinata, Spilonota ocellana, Spilonota lechriaspis, Illiberis pruni, Argyresthia conjugella, Caloptilia zachrysa, Archips breviplicanus, Anomis flava, Pectinophora gossypiella, Notarcha derogata, Diaphania indica, Heliothis virescens and Earias cupreoviridis.

Examples of the species of the order Hemiptera include Nezara antennata, Stenotus rubrovittatus, Graphosoma rubrolineatum, Trigonotylus coelestialium, Aeschynteles maculatus, Creontiades pallidifer, Dysdercus cingulatus, Chrysomphalus ficus, Aonidiella aurantii, Graptopsaltria nigrofuscata, Blissus leucopterus, Icerya purchasi, Piezodorus hybneri, Lagynotomus elongatus, Thaia subrufa, Scotinophara lurida, Sitobion ibarae, Stariodes iwasakii, Aspidiotus destructor, Taylorilygus pallidulus, Myzus mumecola, Pseudaulacaspis prunicola, Acyrthosiphon pisum, Anacanthocoris striicornis, Ectometopterus micantulus, Eysarcoris lewisi, Molipteryx fuliginosa, Cicadella viridis, Rhopalosophum rufiabdominalis, Saissetia oleae, Trialeurodes vaporariorum, Aguriahana quercus, Lygus spp., Euceraphis punctipennis, Andaspis kashicola, Coccus pseudomagnoliarum, Cavelerius saccharivorus, Galeatus spinifrons, Macrosiphoniella sanborni, Aonidiella citrina, Halyomorpha mista, Stephanitis fasciicarina, Trioza camphorae, Leptocorisa chinensis, Trioza quercicola, Uhlerites latius, Erythroneura comes, Paromius exiguus, Duplaspidiotus claviger, Nephotettix nigropictus, Halticiellus insularis, Perkinsiella saccharicida, Psylla malivorella, Anomomeura mori, Pseudococcus longispinis, Pseudaulacaspis pentagona, Pulvinaria kuwacola, Apolygus lucorum, Togo hemipterus, Toxoptera aurantii, Saccharicoccus sacchari, Geoica lucifuga, Numata muiri, Comstockaspis perniciosa, Unaspis citri, Aulacorthum solani, Eysarcoris ventralis, Bemisia argentifolii, Cicadella spectra, Aspidiotus hederae, Liorhyssus hyalinus, Calophya nigridorsalis, Sogatella furcifera, Megoura crassicauda, Brevicoryne brassicae, Aphis glycines, Leptocorisa oratorius, Nephotettix virescens, Uroeucon formosanum, Cyrtopeltis tennuis, Bemisia tabaci, Lecanium persicae, Parlatoria theae, Pseudaonidia paeoniae, Empoasca onukii, Plautia stali, Dysaphis tulipae, Macrosiphum euphorbiae, Stephanitis pyrioides, Ceroplastes ceriferus, Parlatoria camelliae, Apolygus spinolai, Nephotettix cincticeps, Glaucias subpunctatus, Orthotylus flavosparsus, Rhopalosiphum maidis, Peregrinus maidis, Eysarcoris parvus, Cimex lectularius, Psylla abieti, Nilaparvata lugens, Psylla tobirae, Eurydema rugosum, Schizaphis piricola, Psylla pyricola, Parlatoreopsis pyri, Stephanitis nashi, Dysmicoccus wistariae, Lepholeucaspis japonica, Sappaphis piri, Lipaphis erysimi, Neotoxoptera formosana, Rhopalosophum nymphaeae, Edwardsiana rosae, Pinnaspis aspidistrae, Psylla alni, Speusotettix subfusculus, Alnetoidia alneti, Sogatella panicicola, Adelphocoris lineolatus, Dysdercus poecilus, Parlatoria ziziphi, Uhlerites debile, Laodelphax striatellus, Eurydema pulchrum, Cletus trigonus, Clovia punctata, Empoasca sp., Coccus hesperidum, Pachybrachius luridus, Planococcus kraunhiae, Stenotus binotatus, Arboridia apicalis, Macrosteles fascifrons, Dolycoris baccarum, Adelphocoris triannulatus, Viteus vitifolii, Acanthocoris sordidus, Leptocorisa acuta, Macropes obnubilus, Cletus punctiger, Riptortus clavatus, Paratrioza cockerelli, Aphrophora costalis, Lygus disponsi, Lygus saundersi, Crisicoccus pini, Empoasca abietis, Crisicoccus matsumotoi, Aphis craccivora, Megacopta punctatissimum, Eysarcoris guttiger, Lepidosaphes beckii, Diaphorina citri, Toxoptera citricidus, Planococcus citri, Dialeurodes citri, Aleurocanthus spiniferus, Pseudococcus citriculus, Zyginella citri, Pulvinaria citricola, Coccus discrepans, Pseudaonidia duplex, Pulvinaria aurantii, Lecanium corni, Nezara viridula, Stenodema calcaratum, Rhopalosiphum padi, Sitobion akebiae, Schizaphis graminum, Sorhoanus tritici, Brachycaudus helichrysi, Carpocoris purpureipennis, Myzus persicae, Hyalopterus pruni, Aphis farinose yanagicola, Metasalis populi, Unaspis yanonensis, Mesohomotoma camphorae, Aphis spiraecola, Aphis pomi, Lepidosaphes ulmi, Psylla mali, Heterocordylus flavipes, Myzus malisuctus, Aphidonuguis mali, Orientus ishidai, Ovatus malicolens, Eriosoma lanigerum, Ceroplastes rubens and Aphis gossypii.

Examples of the species of the order Coleoptera include Xystrocera globosa, Paederus fuscipes, Eucetonia roelofsi, Callosobruchus chinensis, Cylas formicarius, Hypera postica, Echinocnemus squameus, Oulema oryzae, Donacia provosti, Lissorhoptrus oryzophilus, Colasposoma dauricum, Euscepes postfasciatus, Epilachna varivestis, Acanthoscelides obtectus, Diabrotica virgifera virgifera, Involvulus cupreus, Aulacophora femoralis, Bruchus pisorum, Epilachna vigintioctomaculata, Carpophilus dimidiatus, Cassida nebulosa, Luperomorpha tunebrosa, Phyllotreta striolata, Psacothea hilaris, Aeolesthes chrysothrix, Curculio sikkimensis, Carpophilus hemipterus, Oxycetonia jucunda, Diabrotica spp., Mimela splendens, Sitophilus zeamais, Tribolium castaneum, Sitophilus oryzae, Palorus subdepressus, Melolontha japonica, Anoplophora malasiaca, Neatus picipes, Leptinotarsa decemlineata, Diabrotica undecimpunctata howardi, Sphenophorus venatus, Crioceris quatuordecimpunctata, Conotrachelus nenuphar, Ceuthorhynchidius albosuturalis, Phaedon brassicae, Lasioderma serricorne, Sitona japonicus, Adoretus tenuimaculatus, Tenebrio molitor, Basilepta balyi, Hypera nigrirostris, Chaetocnema concinna, Anomala cuprea, Heptophylla picea, Epilachna vigintioctopunctata, Diabrotica longicornis, Eucetonia pilifera, Agriotes spp., Attagenus unicolor japonicus, Pagria signata, Anomala rufocuprea, Palorus ratzeburgii, Alphitobius laevigatus, Anthrenus verbasci, Lyctus brunneus, Tribolium confusum, Medythia nigrobilineata, Xylotrechus pyrrhoderus, Epitrix cucumeris, Tomicus piniperda, Monochamus alternatus, Popillia japonica, Epicauta gorhami, Sitophilus zeamais, Rhynchites heros, Listroderes costirostris, Callosobruchus maculatus, Phyllobius armatus, Anthonomus pomorum, Linaeidea aenea and Anthonomus grandis.

Examples of the species of the order Diptera include Culex pipiens pallens, Pegomya hyoscyami, Liriomyza huidobrensis, Musca domestica, Chlorops oryzae, Hydrellia sasakii, Agromyza oryzae, Hydrellia griseola, Hydrellia griseola, Ophiomyia phaseoli, Dacus cucurbitae, Drosophila suzukii, Rhacochlaena japonica, Muscina stabulans, the species of the family Phoridae such as Megaselia spiracularis, Clogmia albipunctata, Tipula aino, Phormia regina, Culex tritaeniorhynchus, Anopheles sinensis, Hylemya brassicae, Asphondylia sp., Delia platura, Delia antiqua, Rhagoletis cerasi, Culex pipiens molestus Forskal, Ceratitis capitata, Bradysia agrestis, Pegomya cunicularia, Liriomyza sativae, Liriomyza bryoniae, Chromatomyia horticola, Liriomyza chinensis, Culex quinquefasciatus, Aedes aegypti, Aedes albopictus, Liriomyza trifolii, Liriomyza sativae, Dacus dorsalis, Dacus tsuneonis, Sitodiplosis mosellana, Meromuza nigriventris, Anastrepha ludens and Rhagoletis pomonella.

Examples of the species of the order Hymenoptera include Pristomyrmex pungens, the species of the family Bethylidae, Monomorium pharaonis, Pheidole noda, Athalia rosae, Dryocosmus kuriphilus, Formica fusca japonica, the species of the subfamily Vespinae, Athalia infumata infumata, Arge pagana, Athalia japonica, Acromyrmex spp., Solenopsis spp., Arge mali and Ochetellus glaber.

Examples of the species of the order Orthoptera include Homorocoryphus lineosus, Gryllotalpa sp., Oxya hyla intricata, Oxya yezoensis, Locusta migratoria, Oxya japonica, Homorocoryphus jezoensis and Teleogryllus emma.

Examples of the species of the order Thysanoptera include Selenothrips rubrocinctus, Stenchaetothrips biformis, Haplothrips aculeatus, Ponticulothrips diospyrosi, Thrips flavus, Anaphothrips obscurus, Liothrips floridensis, Thrips simplex, Thrips nigropilosus, Heliothrips haemorrhoidalis, Pseudodendrothrips mori, Microcephalothrips abdominalis, Leeuwenia pasanii, Litotetothrips pasaniae, Scirtothrips citri, Haplothrips chinensis, Mycterothrips glycines, Thrips setosus, Scirtothrips dorsalis, Dendrothrips minowai, Haplothrips niger, Thrips tabaci, Thrips alliorum, Thrips hawaiiensis, Haplothrips kurdjumovi, Chirothrips manicatus, Frankliniella intonsa, Thrips coloratus, Franklinella occidentalis, Thrips palmi, Frankliniella lilivora and Liothrips vaneeckei.

Examples of the species of the order Acari include Leptotrombidium akamushi, Tetranychus ludeni, Dermacentor variabilis, Tetranychus truncatus, Ornithonyssus bacoti, Demodex canis, Tetranychus viennensis, Tetranychus kanzawai, the species of the family Ixodidae such as Rhipicephalus sanguineus, Cheyletus malaccensis, Tyrophagus putrescentiae, Dermatophagoides farinae, Latrodectus hasseltii, Dermacentor taiwanensis, Acaphylla theavagrans, Polyphagotarsonemus latus, Aculops lycopersici, Ornithonyssus sylvairum, Tetranychus urticae, Eriophyes chibaensis, Sarcoptes scabiei, Haemaphysalis longicornis, Ixodes scapularis, Tyrophagus similis, Cheyletus eruditus, Panonychus citri, Cheyletus moorei, Brevipalpus phoenicis, Octodectes cynotis, Dermatophagoides ptrenyssnus, Haemaphysalis flava, Ixodes ovatus, Phyllocoptruta citri, Aculus schlechtendali, Panonychus ulmi, Amblyomma americanum, Dermanyssus gallinae, Rhyzoglyphus robini and Sancassania sp.

Examples of the species of the order Isoptera include Reticulitermes miyatakei, Incisitermes minor, Coptotermes formosanus, Hodotermopsis japonica, Reticulitermes sp., Reticulitermes flaviceps amamianus, Glyptotermes kushimensis, Coptotermes guangzhoensis, Neotermes koshunensis, Glyptotermes kodamai, Glyptotermes satsumensis, Cryptotermes domesticus, Odontotermes formosanus, Glyptotermes nakajimai, Pericapritermes nitobei and Reticulitermes speratus.

Examples of the species of the order Blattodea include Periplaneta fuliginosa, Blattella germanica, Blatta orientalis, Periplaneta brunnea, Blattella lituricollis, Periplaneta japonica and Periplaneta americana.

Examples of the species of the phylum Nematoda include Nothotylenchus acris, Aphelenchoides besseyi, Pratylenchus penetrans, Meloidogyne hapla, Meloidogyne incognita, Globodera rostochiensis, Meloidogyne javanica, Heterodera glycines, Pratylenchus coffeae, Pratylenchus neglectus and Tylenchus semipenetrans.

Examples of the species of the phylum Mollusca include such as Pomacea canaliculata, Achatina fulica, Meghimatium bilineatum, Lehmannina valentiana, Limax flavus and Acusta despecta sieboldiana.

### Pharmaceutical Compositions

The pharmaceutical compositions of the invention comprise effective amounts of compounds of the invention or salts thereof, including those of formulae (I), (Ia) and/or (Ib), in combination with an acceptable carrier or diluent. The pharmaceutical compositions may be in a variety of solid and liquid forms which are suitable for various methods of application or administration to an animal. For example, the pharmaceutical compositions comprising the one or more compounds of the invention may be in compositions suitable for oral administration, injectable administration, including subcutaneous and parenteral administration, topical administration (e.g. spot-on or pour-on), including dermal or subdermal administration.

Suitable dosage forms for oral administration include dietary supplements, troches, lozenges, chewables (e.g. chewable tablets or soft chews), tablets, hard or soft capsules, boluses, emulsions, aqueous or oily suspensions, aqueous or oily solutions, oral drench compositions, dispersible powders or granules, premixes, syrups or elixirs, enteric compositions or pastes. Pharmaceutical compositions intended for oral use may be prepared according to any method known in the art for the manufacture of pharmaceutical compositions. Suitable tablets may be obtained, for example, by mixing one or more compounds of the invention, including those of formulae (I), (Ia) and/or (Ib), with known excipients, for example inert diluents, carriers, disintegrants, adjuvants, surfactants, binders and/or lubricants.

In one embodiment of the invention, a soft chewable veterinary composition is provided comprising an effective amount of at least one compound of formulae (I), (Ia) and/or (Ib) in a pharmaceutically acceptable carrier.

In some embodiments, the compositions may be in the form of a sterile injectable composition. The injectable composition may be a solution or a suspension. This suspension may be formulated according to the known art using those suitable dispersing or wetting agents and suspending agents. The sterile injectable preparation may also be a sterile injectable solution or suspension in a non-toxic parenterally acceptable diluent or solvent.

Implant compositions that may be used to deliver the compounds of the invention are commonly subcutaneously delivered to an animal. In addition, external wearable devices such as collars, pendants, ear tags, and the like, may be used to deliver the compounds of the invention to an animal.

Suitable topical spot-on or pour-on pharmaceutical composition comprise a pharmaceutically effective amount of at least one compound of the invention, including those of formulae (I), (Ia) and/or (Ib), in a pharmaceutically acceptable carrier. The pharmaceutically acceptable carrier for the topical compositions of the invention may include, but are not limited to, solvents and solvent mixtures, permeation enhancers, surfactants, antioxidants, pH stabilizers, preservatives and crystallization inhibitors known in the art.

The agricultural and horticultural insecticidal and acaricidal agent of the present invention is commonly used as a formulation convenient for application, which is prepared by the usual method for preparing agrochemical formulations.

That is, the compound of the formulae (I), (Ia) and/or (Ib) of the present invention or a salt thereof and an appropriate inactive carrier, and if needed an adjuvant, are blended in an appropriate ratio, and through the step of dissolution, separation, suspension, mixing, impregnation, adsorption and/or adhesion, are formulated into an appropriate form for application, such as a suspension concentrate, an emulsifiable concentrate, a soluble concentrate, a wettable powder, a water-dispersible granule, a granule, a dust, a tablet and a pack.

### Combination Therapies

The pharmaceutical compositions comprising the compounds of the invention, including those of formulae (I), (Ia) and/or (Ib), may also include other pharmaceutical active agents.

In one embodiment of the invention, one or more arylpyrazole compounds, such as a phenylpyrazole insecticide known in the art, may be combined with the compounds of the invention, including those of formulae (I), (Ia) and/or (Ib) in the pharmaceutical compositions of the invention. Phenylpyrazole insecticides act by blocking glutamate-activated chloride channels (GABA_{A} gated chloride) in insects.

In another embodiment of the invention, one or more macrocyclic lactones, which act as an acaricide, anthelmintic agent and/or insecticide, can be added to the pharmaceutical compositions of the invention. Macrocyclic lactones include both avermectins and milbemycins active agents.

In another embodiment of the invention, a composition comprising one or more compounds of the invention in combination with one or more molecules of a class of insecticides known as insect growth regulators (IGRs) is provided. Compounds belonging to this group are well known to the practitioner and represent a wide range of different chemical classes. These compounds all act by interfering with the development or growth of the insect pests.

In one embodiment, the IGR is a compound that mimics juvenile hormone (juvenile hormone mimic).

In another embodiment, the IGR compound is a chitin synthesis inhibitor. Chitin synthesis inhibitors act by interfering with the insect molting process.

In yet another embodiment of the invention, adulticide insecticides and acaricides can also be added to the pharmaceutical compositions of the invention. In some embodiments, the pharmaceutical compositions of the invention may include one or more antinematodal agents. Antinematodal agents (e.g. nematicides or nematocides) are active against parasitic worms such as roundworms.

In other embodiments, the pharmaceutical compositions of the invention may include one or more antitrematodal agents. Antitrematodal agents are active against trematodes, including parasitic flatworms known as flukes such as Clonorhis sinensis, Fasciola hepatica and Opisthorchis species.

One or more anticestodal compounds may also be advantageously used in the pharmaceutical compositions of the invention. Anticestodal compounds are active agents that are active against cestodes known as tapeworms.

In yet other embodiments, the pharmaceutical compositions of the invention may include one or more other active agents that are effective against arthropod parasites.

An antiparasitic agent that can be combined with one or more compounds of the invention to form a pharmaceutical composition can be one or more biologically active peptides or proteins including, but not limited to, anthelmintic cyclic depsipeptides, which act at the neuromuscular junction by stimulating presynaptic receptors belonging to the secretin receptor family resulting in the paralysis and death of parasites. Anthelmintic cyclic depsipeptides include anthelmintic cyclooctadepsipeptides known in the art.

In another embodiment, the pharmaceutical compositions of the invention may comprise one or more active agents from the neonicotinoid class of pesticides. The neonicotinoids bind and inhibit insect specific nicotinic acetylcholine receptors.

In another embodiment, the compositions of the invention may advantageously include one or more insecticidal and/or isoxazoline active agents known in the art that are potent inhibitors of gamma-aminobutyric acid (GABA)-gated chloride channels. Isoxazoline active agents are, among others, highly effective against ectoparasites, such as fleas and ticks.

In another embodiment of the invention, nodulisporic acid and/or its derivatives (a class of known acaricidal, anthelmintic, anti-parasitic and insecticidal agents) may be added to the pharmaceutical compositions of the invention.

In another embodiment, one or more anthelmintic compounds of the amino acetonitrile class (AAD) of compounds may be added to the pharmaceutical compositions of the invention. In another embodiment, one or more aryloazol-2-yl cyanoethylamino compounds may be included in the pharmaceutical compositions.

The pharmaceutical compositions of the invention may also be combined with one or more paraherquamide compounds and/or derivatives of these compounds. The paraherquamide family of compounds is a known class of compounds that include a spirodioxepino indole core with activity against certain parasites. In addition, the structurally related marcfortine family of compounds are also known and may be combined with the pharmaceutical compositions of the invention.

In another embodiment of the invention, the pharmaceutical compositions may include one or more spinosyn active agents produced by the soil actinomycete Saccharopolyspora spinosa or a semi-synthetic spinosoid active agent. The spinosyns are typically referred to as factors or components A, B, C, D, E, F, G, H, J, K, L, M, N, O, P, Q, R, S, T, U, V, W, or Y, and any of these components, or a combination thereof, may be used in the pharmaceutical compositions of the invention.

In one embodiment of the invention, the compositions of the invention may also include the agricultural and horticultural insecticidal and acaricidal agent of the present invention combined with one or more compounds selected from th group consisting of: acetylcholinesterase (ACHE) inhibitors, baculoviruses, calcium-activated potassium-channel (KCA2) modulators, chordotonal organ modulators (undefined target size), chordotonal organ TRPV channel modulators, ecdysone receptor agonists, GABA-gated chloride channel allosteric modulators, GABA-gated chloride channel blockers, glutamate-gated chlorine channel (GLUCL) allosteric modulators, inhibitors of acetyl CoA carboxylase, inhibitors of chitin biosynthesis affecting CHS1, inhibitors of chitin biosynthesis type 1, inhibitors of mitochondrial ATP synthase, juvenile hormone mimics, microbial disruptors of insect midgut membranes, mite growth inhibitors affecting CHS1, mitochondrial complex I electron transport inhibitors, mitochondrial complex II electron transport inhibitors, mitochondrial complex III electron transport inhibitors QI site, mitochondrial complex III electron transport inhibitors QO site, mitochondrial complex IV electron transport inhibitors, moulting disruptor, nicotinic acetylcholine receptor (NACHR) allosteric modulators—site I, nicotinic acetylcholine receptor (NACHR) allosteric modulators—site II, nicotinic acetylcholine receptor (NACHR) channel blockers, nicotinic acetylcholine receptor (NACHR) competitive modulators, octopamine receptor agonists, ryanodine receptor modulators, sodium channel modulators, uncouplers of oxidative phosphorylation via disruption of the proton gradient, voltage-dependent sodium channel blockers.

### Chemical Synthesis

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Starting materials may be prepared by methods that are described in the literature or herein, or may be prepared in an analogous or similar manner. Any functional groups in the starting materials or intermediates may be protected using conventional protecting groups. These protecting groups may be cleaved again at a suitable stage within the reaction sequence using methods familiar to the one skilled in the art.

The Examples that follow are intended to illustrate the present invention without restricting it. The terms “ambient temperature” and “room temperature” are used interchangeably and designate a temperature of about 20° C. The compounds of formulae (I), (Ia) and (Ib) or pharmaceutically acceptable salts thereof may be prepared by adopting one of the following reaction schemes. The starting materials for their preparation can be prepared by methods known per se and as described in the literature or are an intermediate of any of the other schemes detailed herein. Although the following subject matter is described in some detail by way of illustration and example for purposes of clarity of understanding, it will be understood by those skilled in the art that certain changes and modifications can be practiced within the scope of the Examples.

### Abbreviations

| | |
|---|---|
| ACN | acetonitrile |
| Alox | aluminium oxide |
| Aq. | aqueous |
| ° C. | degree celsius |
| CyH/CH | cyclohexane |
| conc. | concentrated |
| DCC | N,N′-dicyclohexylmethanediimine |
| DCM | dichloro methane |
| 1,2-DCE | 1,2-dichloro ethane |
| DEE | diethyl ether |
| DIPE | diisopropylether |
| DIPEA | N,N-diisopropylethylamine |
| 1,2-DME | 1,2-dimethyl ether |
| DMF | N,N-dimethylformamide |
| DMSO | dimethyl sulfoxide |
| ESI-MS | electrospray ionisation mass spectrometry |
| Et₂O | diethylether |
| EtOAc/EE/EA | ethyl acetate |
| ex | example |
| eq | equivalent |
| h | hour |
| HAc | acetic acid |
| HATU | N,N,N′,N′-tetramethyl-O-(7-azabenzotriazol-1-yl)uranium hexafluorophosphate |
| HCl | hydrochlorid acid |
| HNO₃ | nitric acid |
| HOAc | acetic acid |
| HPLC | high performance liquid chromatography |
| K₃PO₄ | tripotassium phosphate |
| LED | light emitting diode |
| LiHMDS | lithium-bis(trimethylsilyl)amide |
| LiOH | lithium hydroxide |
| LR | Lawesson's reagent |
| MeOH | methanol |
| NaH | sodium hydride |
| NaHCO₃ | sodium bicarbonate |
| Na₂SO₄ | sodium sulfate |
| NBS | N-bromosuccinimide |
| NCIS | N-chlorosuccinimide |
| NH₃ | ammonia |
| NH₄Cl | ammonium chloride |
| NIS | N-iodosuccinimide |
| NMP | N-methyl-2-pyrrolidone |
| min | minute |
| mL | milliliter |
| Pd/C | palladium on activated carbon |
| Pd(dppf)Cl2 | [1,1′-bis(diphenylphosphino)ferrocene]dichloropalladium(II) |
| PE | petroleum ether |
| PtO₂ | platinum dioxide |
| RT | room temperature (about 20° C.) |
| Rₜ | retention time |
| sat. | saturated |
| TEA | triethylamine |
| TFA | trifluoroacetic acid |
| THF | tetrahydrofuran |
| TLC | thin-layer chromatography on SiO₂ |

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Chemical Syntheses

### Synthesis of 5-bromo-3-(ethanesulfonyl)-2-[17-methyl-3-(1,1,2,2,2-pentafluoroethyl) imidazo[4,5-c]pyridazin-6-yl]pyridine

### Synthesis of N-methyl-6-(1,1,2,2,2-pentafluoroethyl)pyridazin-3-amine

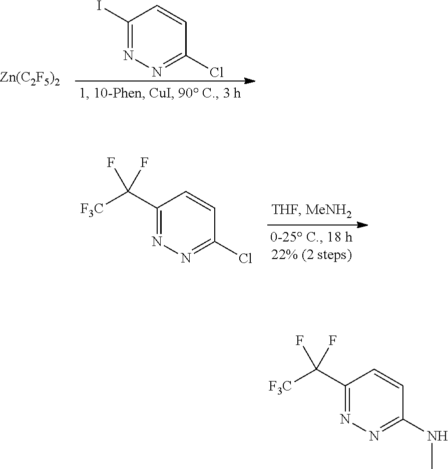

Into a 5 L 3-necked round-bottom flask purged and maintained with an inert atmosphere of nitrogen, was placed a solution of bis(1,1,2,2,2-pentafluoroethyl)zinc (60 g, 198 mmol, 1.0 eq) in NMP (2 mL), CuI (19 g, 99 mmol, 0.5 eq), 1,10-phenanthroline (18 g, 99 mmol, 0.5 eq), 3-chloro-6-iodopyridazine (48 g, 198 mmol, 1 eq). The resulting solution was stirred for 3 h at 90 degrees C. The mixture was used in the next step without any other purification.

Into a 5-L 3-necked round-bottom flask, was placed a solution of 3-chloro-6-(1,1,2,2,2-pentafluoroethyl)pyridazine (60 g, 258 mmol, 1.0 eq) in NMP (2 L), THF (80 mL). This was followed by the addition of MeNH₂ in EtOH (200 mL, 10 eq, 35%) at 0 degrees C. The resulting solution was stirred for 18 h at 25 degrees C. The solids were filtered out. The resulting solution was extracted with 3×800 mL of ethyl acetate. The organic layer was washed with 5×800 mL of brine. The resulting mixture was concentrated. The residue was applied onto a silica gel column with ethyl acetate/petroleum ether (1:1). This resulted in 13 g (22%) of N-methyl-6-(1,1,2,2,2-pentafluoroethyl)pyridazin-3-amine as a yellow solid.

### Synthesis of 4-bromo-N-methyl-6-(1,1,2,2,2-pentafluoroethyl)pyridazin-3-amine

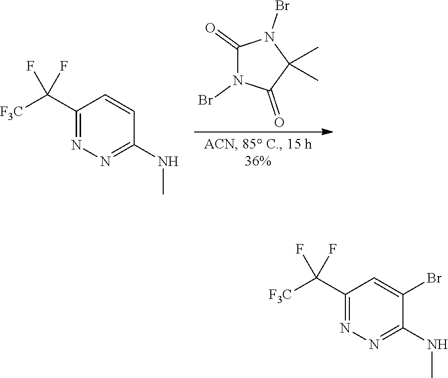 [SMILES: CNc1ccc(C(F)(F)C(F)(F)F)nn1 ; CC1(C)C(=O)N(Br)C(=O)N1Br ; CNc1nnc(C(F)(F)C(F)(F)F)cc1Br]

Into a 1 L round-bottom flask, was placed a solution of N-methyl-6-(1,1,2,2,2-pentafluoroethyl)pyridazin-3-amine (29 g, 128 mmol, 1.0 eq) in ACN (400 mL), 1,3-dibromo-5,5-dimethylhydantoin (80 g, 281 mmol, 2.2 eq). The resulting solution was stirred for 15 h at 85 degrees C. The reaction was then quenched by the addition of 500 mL of 10% NaHSO₃. The resulting solution was extracted with 2×500 mL of ethyl acetate. The resulting mixture was washed with 2×500 mL of brine. The resulting mixture was concentrated. The residue was applied onto a silica gel column with ethyl acetate/petroleum ether (1:5-1:1). This resulted in 14 g (36%) of 4-bromo-N-methyl-6-(1,1,2,2,2-pentafluoroethyl)pyridazin-3-amine as a light yellow solid.

### Synthesis of N3-methyl-6-(1,1,2,2,2-pentafluoroethyl)pyridazine-3,4-diamine

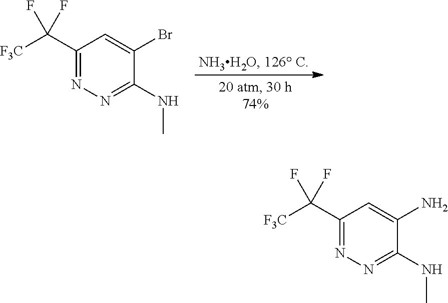 [SMILES: CNc1nnc(C(F)(F)C(F)(F)F)cc1Br ; CNc1nnc(C(F)(F)C(F)(F)F)cc1N]

Into a 1 L pressure tank reactor purged and maintained with an inert atmosphere of nitrogen, was placed 4-bromo-N-methyl-6-(1,1,2,2,2-pentafluoroethyl)pyridazin-3-amine (17 g, 55 mmol, 1.0 equiv), NH₃·H₂O (300 mL). The resulting solution was stirred for 30 h at 126 degrees C. under 20 atm N₂ atmosphere. The resulting mixture was concentrated. The residue was applied onto a silica gel column with ethyl acetate/petroleum ether (1:0). This resulted in 10 g (74%) of N3-methyl-6-(1,1,2,2,2-pentafluoroethyl)pyridazine-3,4-diamine as a yellow solid.

### Synthesis of 5-bromo-3-(ethylsulfanyl)-N-[3-(methylamino)-6-(1,1,2,2,2-pentafluoroethyl)pyridazin-4-yl]pyridine-2-carboxamide

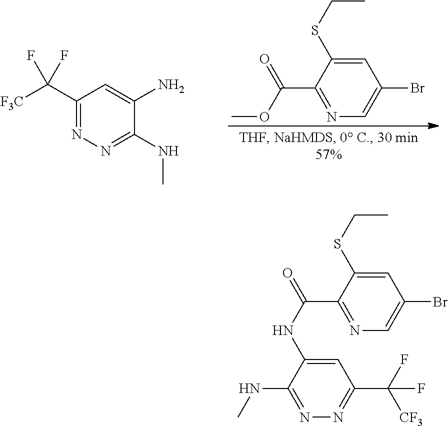 [SMILES: CNc1nnc(C(F)(F)C(F)(F)F)cc1N ; CCSc1cc(Br)cnc1C(=O)OC ; CCSc1cc(Br)cnc1C(=O)Nc1cc(C(F)(F)C(F)(F)F)nnc1NC]

Into a 250 mL round-bottom flask purged and maintained with an inert atmosphere of nitrogen, was placed a solution of methyl 5-bromo-3-(ethylsulfanyl)pyridine-2-carboxylate (9 g, 33 mmol, 1.0 eq) in THF (150 mL), N3-methyl-6-(1,1,2,2,2-pentafluoroethyl)pyridazine-3,4-diamine (8.3 g, 34 mmol, 1.0 eq). This was followed by the addition of NaHMDS (29 mL, 57 mmol, 1.8 eq, 2M) dropwise with stirring at 0 degrees C. The resulting solution was stirred for 30 min at 0 degrees C. The reaction was then quenched by the addition of 500 mL of 10% NH₄Cl. The resulting solution was extracted with 2×250 mL of ethyl acetate. The organic layer was concentrated, applied onto a silica gel column with ethyl acetate/petroleum ether (1:10-1:3). This resulted in 9 g (57%) of 5-bromo-3-(ethylsulfanyl)-N-[3-(methylamino)-6-(1,1,2,2,2-pentafluoroethyl)pyridazin-4-yl]pyridine-2-carboxamide as a yellow solid.

### Synthesis of 5-bromo-3-(ethylsulfanyl)-2-[7-methyl-3-(1,1,2,2,2-pentafluoroethyl)-7H-imidazo[4,5-c]pyridazin-6-yl]pyridine

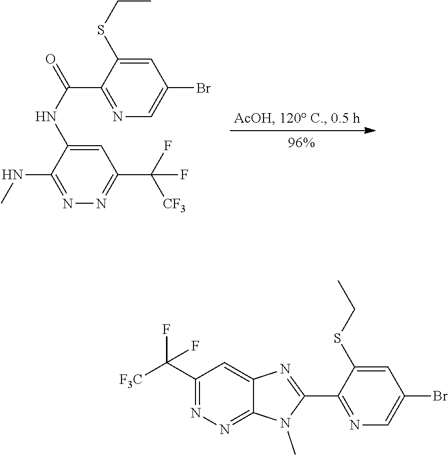 [SMILES: CCSc1cc(Br)cnc1C(=O)Nc1cc(C(F)(F)C(F)(F)F)nnc1NC ; CCSc1cc(Br)cnc1-c1nc2cc(C(F)(F)C(F)(F)F)nnc2n1C]

Into a 250 mL round-bottom flask, was placed 5-bromo-3-(ethylsulfanyl)-N-[3-(methylamino)-6-(1,1,2,2,2-pentafluoroethyl)pyridazin-4-yl]pyridine-2-carboxamide (6.5 g, 13.4 mmol, 1.0 eq), AcOH (100 mL). The resulting solution was stirred for 0.5 h at 120 degrees C. The resulting mixture was concentrated. The resulting solution was diluted with 200 mL of EA. The resulting mixture was washed with 2×50 mL of 10% NaHCO₃. The resulting mixture was washed with 2×50 mL of brine. The resulting mixture was concentrated. The residue was applied onto a silica gel column with ethyl acetate/petroleum ether (1:10-1:3). This resulted in 6 g (96%) of 5-bromo-3-(ethylsulfanyl)-2-[7-methyl-3-(1,1,2,2,2-pentafluoroethyl)imidazo[4,5-c]pyridazin-6-yl]pyridine as a light yellow solid.

### Synthesis of 5-bromo-3-(ethanesulfonyl)-2-[7-methyl-3-(1,1,2,2,2-pentafluoroethyl)-7H-imidazo[4,5-c]pyridazin-6-yl]pyridine

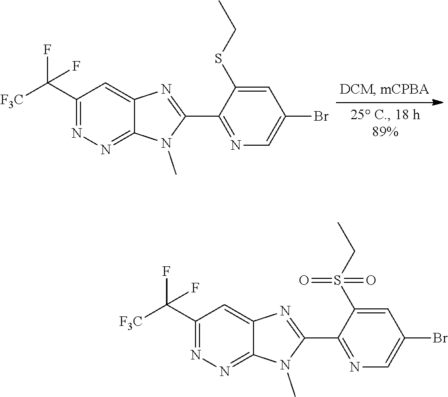 [SMILES: CCSc1cc(Br)cnc1-c1nc2cc(C(F)(F)C(F)(F)F)nnc2n1C ; CCS(=O)(=O)c1cc(Br)cnc1-c1nc2cc(C(F)(F)C(F)(F)F)nnc2n1C]

Into a 250-mL round-bottom flask, was placed a solution of 5-bromo-3-(ethylsulfanyl)-2-[7-methyl-3-(1,1,2,2,2-pentafluoroethyl)imidazo[4,5-c]pyridazin-6-yl]pyridine (8.0 g, 17 mmol, 1.0 eq) in DCM (180 mL). This was followed by the addition of mCPBA (12 g, 68 mmol, 4.0 eq) at 0 degrees C. The resulting solution was stirred for 10 h at 0-25 degrees C. The resulting mixture was concentrated. The residue was applied onto a silica gel column with ethyl acetate/petroleum ether (1:4). This resulted in 7.6 g (89%) of 5-bromo-3-(ethanesulfonyl)-2-[7-methyl-3-(1,1,2,2,2-pentafluoroethyl) imidazo[4,5-c]pyridazin-6-yl]pyridine as a yellow solid.

### Synthesis of 6-[5-(1-cyclopropyltetrazol-5-yl)-3-ethylsulfonyl-2-pyridyl]-7-methyl-3-(1,1,2,2,2-pentafluoroethyl)imidazo[4,5-c]pyridazine (1)

Specifically, the following compounds of formula (I), formula (Ia) and formula (Ib) can be synthesized by adopting the subsequent scheme of Compound 1 by someone who is skilled in the art and employing the starting material described vide supra: 3, 4, 5, 6, 11, 13, 15, 16:

### Synthesis of methyl 5-ethylsulfonyl-6-[7-methyl-3-(1,1,2,2,2-pentafluoroethyl)imidazo[4,5-c]pyridazin-6-yl]pyridine-3-carboxylate

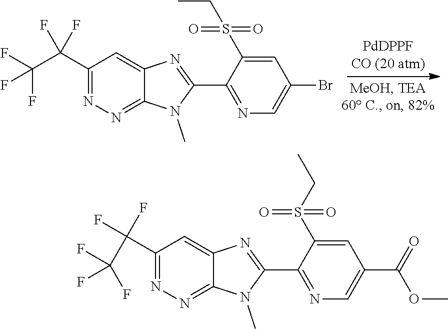 [SMILES: CCS(=O)(=O)c1cc(Br)cnc1-c1nc2cc(C(F)(F)C(F)(F)F)nnc2n1C ; CCS(=O)(=O)c1cc(C(=O)OC)cnc1-c1nc2cc(C(F)(F)C(F)(F)F)nnc2n1C]

To a solution of 6-(5-bromo-3-ethylsulfonyl-2-pyridyl)-7-methyl-3-(1,1,2,2,2-pentafluoroethyl)imidazo[4,5-c]pyridazine (1.0 eq, 3.2 g, 6.4 mmol), TEA (3.0 eq, 2.7 mL, 19 mmol) in MeOH (150 mL) was added [1,1′-bis(diphenylphosphino)ferrocene]dichloropalladium(II), complex with dichloromethane (0.1 eq, 0.5 g, 0.64 mmol) in a pressure tank. The mixture was purged with nitrogen for 10 min and then was pressurized to 20 atm with carbon monoxide at 60° C. for overnight. The reaction mixture was cooled to room temperature The resulting mixture was concentrated under reduced pressure. The residue was purified by silica gel column chromatography, eluted with PE/THF (1:1) to afford methyl 5-ethylsulfonyl-6-[7-methyl-3-(1,1,2,2,2-pentafluoroethyl)imidazo[4,5-c]pyridazin-6-yl]pyridine-3-carboxylate (2.8 g, 82%) as a white solid.

### Synthesis of in 5-ethylsulfonyl-6-[7-methyl-3-(1,1,2,2,2-pentafluoroethyl)imidazo[4,5-c]pyridazin-6-yl]pyridine-3-carboxylic acid

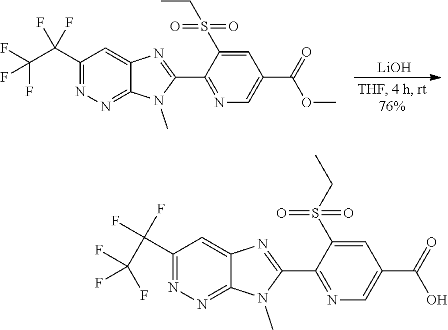 [SMILES: CCS(=O)(=O)c1cc(C(=O)OC)cnc1-c1nc2cc(C(F)(F)C(F)(F)F)nnc2n1C ; "LiOH" ; CCS(=O)(=O)c1cc(C(=O)O)cnc1-c1nc2cc(C(F)(F)C(F)(F)F)nnc2n1C]

Into a 250 mL round-bottom flask were added methyl 5-ethylsulfonyl-6-[7-methyl-3-(1,1,2,2,2-pentafluoroethyl)imidazo[4,5-c]pyridazin-6-yl]pyridine-3-carboxylate (1.0 eq, 2.8 g, 5.84 mmol), LiOH·H₂O (5.0 eq, 1.2 g, 29 mmol), THF (100 mL) and H₂O (20 mL). The mixture was stirred for 4 h at room temperature. The mixture was acidified to pH 3 with conc. HCl. The precipitated solids were collected by filtration and washed with water (2×50 mL). The resulting solid was dried under infrared light. This resulted in 5-ethylsulfonyl-6-[7-methyl-3-(1,1,2,2,2-pentafluoroethyl)imidazo[4,5-c]pyridazin-6-yl]pyridine-3-carboxylic acid (2.3 g, 76%) as a white solid.

### Synthesis of in 5-ethylsulfonyl-6-[7-methyl-3-(1,1,2,2,2-pentafluoroethyl)imidazo[4,5-c]pyridazin-6-yl]pyridine-3-carbonyl chloride

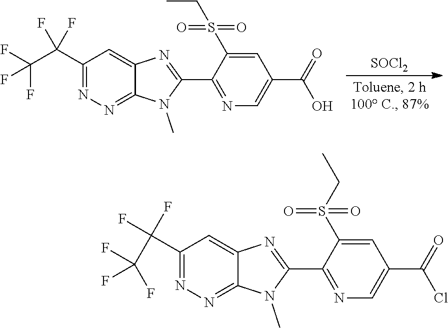 [SMILES: CCS(=O)(=O)c1cc(C(=O)O)cnc1-c1nc2cc(C(F)(F)C(F)(F)F)nnc2n1C ; "SOCl2" ; CCS(=O)(=O)c1cc(C(=O)Cl)cnc1-c1nc2cc(C(F)(F)C(F)(F)F)nnc2n1C]

Into a 500 mL round-bottom flask were added 5-ethylsulfonyl-6-[7-methyl-3-(1,1,2,2,2-pentafluoroethyl)imidazo[4,5-c]pyridazin-6-yl]pyridine-3-carbonyl chloride (2.0 g, 3.7 mmol, 1.0 eq), SOCl₂ (10 eq, 3.1 mL, 43 mmol) and toluene (80 mL). The mixture was stirred for 2 h at 100° C. The mixture was allowed to cool down to room temperature. The resulting mixture was concentrated under reduced pressure. This resulted in 5-ethylsulfonyl-6-[7-methyl-3-(1,1,2,2,2-pentafluoroethyl)imidazo[4,5-c]pyridazin-6-yl]pyridine-3-carbonyl chloride (2.0 g, 87%) as a white solid.

### Synthesis of in N-cyclopropyl-5-ethylsulfonyl-6-[7-methyl-3-(1,1,2,2,2-pentafluoroethyl)imidazo[4,5-c]pyridazin-6-yl]pyridine-3-carboxamide

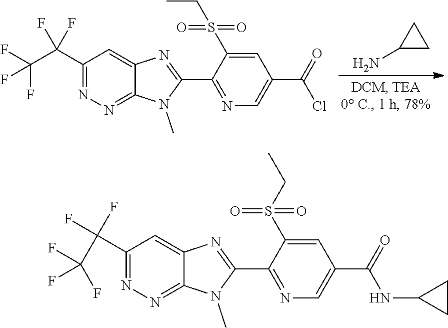

Into a 250 mL round-bottom flask were added 5-ethylsulfonyl-6-[7-methyl-3-(1,1,2,2,2-pentafluoroethyl)imidazo[4,5-c]pyridazin-6-yl]pyridine-3-carbonyl chloride (1.0 eq, 2.0 g, 4.1 mmol), cyclopropanamine (1.2 eq, 0.28 g, 5.0 mmol), TEA (4.0 eq, 2.3 mL, 17 mmol) and DCM (100 mL). The mixture was stirred for 1 h at 0° C. The reaction was quenched by the addition of water (100 mL). The resulting mixture was extracted with CH₂Cl₂ (2×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/THF (1:1) to afford N-cyclopropyl-5-ethylsulfonyl-6-[7-methyl-3-(1,1,2,2,2-pentafluoroethyl)imidazo[4,5-c]pyridazin-6-yl]pyridine-3-carboxamide (1.8 g, 78%) as a yellow solid.

### Synthesis of in 6-[5-(1-cyclopropyltetrazol-5-yl)-3-ethylsulfonyl-2-pyridyl]-7-methyl-3-(1,1,2,2,2-pentafluoroethyl)imidazo[4,5-c]pyridazine

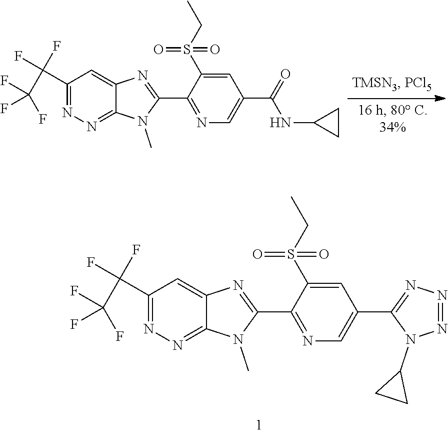 [SMILES: CCS(=O)(=O)c1cc(C(=O)NC2CC2)cnc1-c1nc2cc(C(F)(F)C(F)(F)F)nnc2n1C ; CCS(=O)(=O)c1cc(-c2nnnn2C2CC2)cnc1-c1nc2cc(C(F)(F)C(F)(F)F)nnc2n1C]

Into a 250 mL round-bottom flask were added N-cyclopropyl-5-ethylsulfonyl-6-[7-methyl-3-(1,1,2,2,2-pentafluoroethyl)imidazo[4,5-c]pyridazin-6-yl]pyridine-3-carboxamide (1.0 eq, 750 mg, 1.5 mmol) and Phosphorus pentachloride (4.0 eq, 1237 mg, 6.0 mmol). The mixture was stirred for 2 h at 80° C. Added TMSN₃ (10 eq, 2.5 mL, 15 mmol). The mixture was stirred for overnight at 80° C. The mixture was allowed to cool down to room temperature. The reaction was quenched by the addition of water/ice (100 mL) at 0° C. The resulting mixture was extracted with CH₂Cl₂ (3×100 mL). The combined organic layers were washed with brine (2×50 mL), dried over anhydrous Na₂SO₄. After filtration, the filtrate was concentrated under reduced pressure. The crude product was purified by Prep-HPLC with the following conditions (Column, C18 silica gel; mobile phase, CH₃CN/0.05% NH₃·H₂O=70%; Detector, 254&220 nm) to afford 6-[5-(1-cyclopropyltetrazol-5-yl)-3-ethylsulfonyl-2-pyridyl]-7-methyl-3-(1,1,2,2,2-pentafluoroethyl)imidazo[4,5-c]pyridazine (268 mg, 34%) as a white solid.

### Synthesis of 6-[3-ethylsulfonyl-5-(2-methyltetrazol-5-yl)-2-pyridyl]-7-methyl-3-(1,1,2,2,2-pentafluoroethyl)imidazo[4,5-c]pyridazine (7)

Specifically, the following compounds of formula (I), formula (Ia) and formula (Ib) can be synthesized by adopting the subsequent scheme of Compound 7 by someone who is skilled in the art and employing the starting material described vide supra: 8, 9, 10, 14:

### Synthesis of 5-(ethanesulfonyl)-6-[7-methyl-3-(1,1,2,2,2-pentafluoroethyl)imidazo[4,5-c]pyridazin-6-yl]pyridine-3-carbonitrile

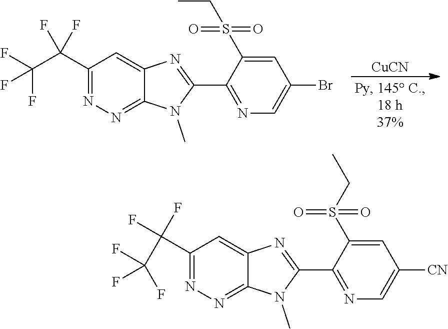

Into a 100 mL sealed tube, was placed a solution of 5-bromo-3-(ethanesulfonyl)-2-[7-methyl-3-(1,1,2,2,2-pentafluoroethyl)imidazo[4,5-c]pyridazin-6-yl]pyridine (3.5 g, 7.1 mmol, 1.0 eq) in NMP (20 mL), pyridine (20 mL), CuCN (6.3 g, 70 mmol, 10 eq). The resulting solution was stirred for 18 h at 145 degrees C. The resulting mixture was concentrated. 500 mL of EA was added in. The resulting mixture was washed with 3×100 mL of brine. The organic layer was concentrated, applied onto a silica gel column with ethyl acetate/petroleum ether (1:10-1:2). This resulted in 1.15 g (37%) of 5-(ethanesulfonyl)-6-[7-methyl-3-(1,1,2,2,2-pentafluoroethyl)imidazo[4,5-c]pyridazin-6-yl]pyridine-3-carbonitrile as a yellow solid.

### Synthesis of methyl 6-[3-ethylsulfonyl-5-(2H-tetrazol-5-yl)-2-pyridyl]-7-methyl-3-(1,1,2,2,2-pentafluoroethyl)imidazo[4,5-c]pyridazine

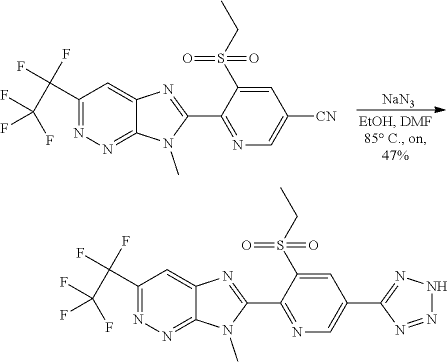

Into a mL round-bottom flask were added 5-ethylsulfonyl-6-[7-methyl-3-(1,1,2,2,2-pentafluoroethyl)imidazo[4,5-c]pyridazin-6-yl]pyridine-3-carbonitrile (1.0 eq, 1.6 g, 3.6 mmol), EtOH (16 mL), DMF (16 mL) and NaN₃ (5.0 eq, 1.2 g, 18 mmol) at room temperature. The resulting mixture was stirred for overnight at 85° C. The mixture was allowed to cool down to room temperature. The resulting mixture was diluted with water (200 mL). The resulting mixture was extracted with EtOAc (3×100 mL). The combined organic layers were washed with brine (3×400 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.05% NH₃·H₂O), 10% to 50% gradient in 10 min; detector, UV 254 nm. This resulted in 6-[3-ethylsulfonyl-5-(2H-tetrazol-5-yl)-2-pyridyl]-7-methyl-3-(1,1,2,2,2-pentafluoroethyl)imidazo[4,5-c]pyridazine (860 mg, 47%) as a yellow solid.

### Synthesis of methyl 6-[3-ethylsulfonyl-5-(2-methyltetrazol-5-yl)-2-pyridyl]-7-methyl-3-(1,1,2,2,2-pentafluoroethyl)imidazo[4,5-c]pyridazine

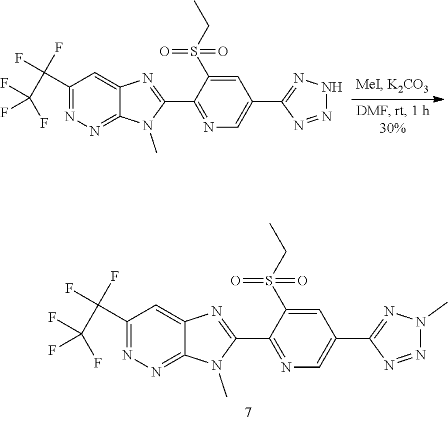 [SMILES: CCS(=O)(=O)c1cc(-c2nn[nH]n2)cnc1-c1nc2cc(C(F)(F)C(F)(F)F)nnc2n1C ; CCS(=O)(=O)c1cc(-c2nnn(C)n2)cnc1-c1nc2cc(C(F)(F)C(F)(F)F)nnc2n1C]

Into a 50 mL round-bottom flask were added 6-[3-ethylsulfonyl-5-(2H-tetrazol-5-yl)-2-pyridyl]-7-methyl-3-(1,1,2,2,2-pentafluoroethyl)imidazo[4,5-c]pyridazine (1.0 eq, 860 mg, 1.8 mmol), DMF (15 mL), CH₃I (1.5 eq, 374 mg, 2.6 mmol) and K₂CO₃ (3.0 eq, 729 mg, 5.3 mmol) at room temperature. The resulting mixture was stirred for 1 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. Column: CHIRALPAK IG, 3*25 cm, 5 μm; Mobile Phase A: CO₂, Mobile Phase B: MeOH (0.1% 2M NH₃-MeOH); Flow rate: 80 mL/min; Gradient: isocratic 40% B; Column Temperature (° C.): 35; Back Pressure (bar): 100; Wave Length: 220 nm; RT1 (min): 3; RT2 (min): 5.4; Sample Solvent: MeOH:DCM=2:1; Injection Volume: 9 mL; This resulted in 6-[3-ethylsulfonyl-5-(2-methyltetrazol-5-yl)-2-pyridyl]-7-methyl-3-(1,1,2,2,2-pentafluoroethyl)imidazo[4,5-c]pyridazine (262 mg, 30%) as a yellow solid.

### Synthesis of 6-[5-(5-cyclopropyltetrazol-1-yl)-3-ethylsulfonyl-2-pyridyl]-7-methyl-3-(1,1,2,2,2-pentafluoroethyl)imidazo[4,5-c]pyridazine (2)

Specifically, the following compounds of formula (I), formula (Ia) and formula (Ib) can be synthesized by adopting the subsequent scheme of Compound 2 by someone who is skilled in the art and employing the starting material described vide supra: 12:

### Synthesis of methyl tert-butyl N-[5-(ethanesulfonyl)-6-[7-methyl-3-(1,1,2,2,2-pentafluoroethyl)-7H-imidazo[4,5-c]pyridazin-6-yl]pyridin-3-yl]carbamate

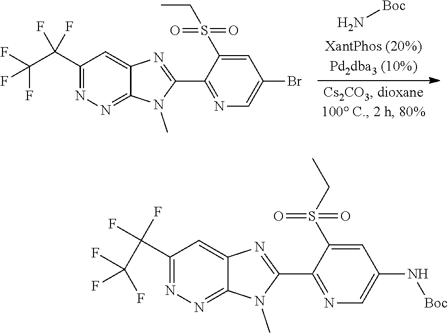 [SMILES: CCS(=O)(=O)c1cc(Br)cnc1-c1nc2cc(C(F)(F)C(F)(F)F)nnc2n1C ; CC(C)(C)OC(N)=O ; CCS(=O)(=O)c1cc(NC(=O)OC(C)(C)C)cnc1-c1nc2cc(C(F)(F)C(F)(F)F)nnc2n1C]

To a solution of 6-(5-bromo-3-ethylsulfonyl-2-pyridyl)-7-methyl-3-(1,1,2,2,2-pentafluoroethyl)imidazo[4,5-c]pyridazine (1.0 eq, 350 mg, 0.7 mmol) and calcium carbonate (2.0 eq, 456 mg, 1.4 mmol) in dioxane (15 mL) was added a solution of Pd₂dba₃ (0.1 eq, 64 mg, 0.1 mmol)) and XantPhos (0.2 eq, 81 mg, 0.1 mmol) in dioxane (5 mL) followed by tert-butyl carbamate (2.0 eq, 164 mg, 1.4 mmol). The resulting mixture was stirred at 100° C. for two hours. The reaction mixture was cooled to room temperature The resulting mixture was concentrated under reduced pressure. The residue was purified by silica gel column chromatography, eluted with PE/THF (1:1) to afford tert-butyl N-[5-(ethanesulfonyl)-6-[7-methyl-3-(1,1,2,2,2-pentafluoroethyl)-7H-imidazo[4,5-c]pyridazin-6-yl]pyridin-3-yl]carbamate (300 mg, 80%) as a white solid.

### Synthesis of tert-butyl N-cyclopropanecarbonyl-N-[5-(ethanesulfonyl)-6-[7-methyl-3-(1,1,2,2,2-pentafluoroethyl)imidazo[4,5-c]pyridazin-6-yl]pyridin-3-yl]carbamate

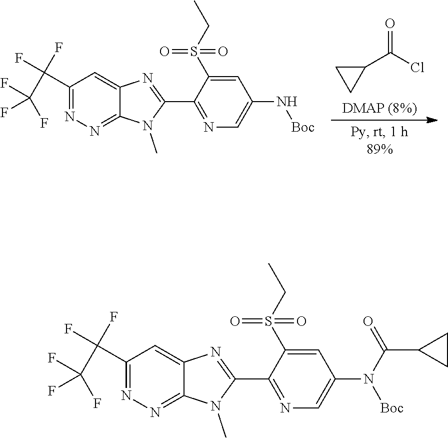 [SMILES: CCS(=O)(=O)c1cc(NC(=O)OC(C)(C)C)cnc1-c1nc2cc(C(F)(F)C(F)(F)F)nnc2n1C ; O=C(Cl)C1CC1 ; CCS(=O)(=O)c1cc(N(C(=O)OC(C)(C)C)C(=O)C2CC2)cnc1-c1nc2cc(C(F)(F)C(F)(F)F)nnc2n1C]

Into a 40 mL vial were added tert-butyl N-(cyclopropanecarbonyl)-N-[5-ethylsulfonyl-6-[7-methyl-3-(1,1,2,2,2-pentafluoroethyl)imidazo[4,5-c]pyridazin-6-yl]-3-pyridyl]carbamate (1.0 eq, 270 mg, 0.4 mmol), pyridine (8 mL), cyclopropanecarbonyl chloride (1.5 eq, 79 mg, 0.8 mmol) and DMAP (0.1 eq, 5.0 mg, 0.04 mmol) at room temperature. The resulting mixture was stirred for 1 h at room temperature. The residue was purified by reverse flash chromatography with the following conditions: column, C18 silica gel; mobile phase, MeCN in Water (0.1% NH₃·H₂O), 85% gradient in 10 min; detector, UV 254 nm. This resulted in tert-butyl N-cyclopropanecarbonyl-N-[5-(ethanesulfonyl)-6-[7-methyl-3-(1,1,2,2,2-pentafluoroethyl)imidazo[4,5-c]pyridazin-6-yl]pyridin-3-yl]carbamate (270 mg, 89%) as a yellow solid.

### Synthesis of N-[5-ethylsulfonyl-6-[7-methyl-3-(1,1,2,2,2-pentafluoroethyl)imidazo[4,5-c]pyridazin-6-yl]-3-pyridyl]cyclopropanecarboxamide

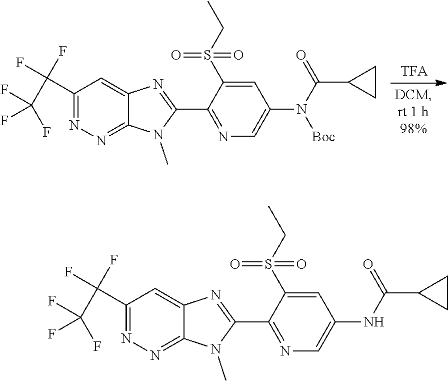 [SMILES: CCS(=O)(=O)c1cc(N(C(=O)OC(C)(C)C)C(=O)C2CC2)cnc1-c1nc2cc(C(F)(F)C(F)(F)F)nnc2n1C ; CCS(=O)(=O)c1cc(NC(=O)C2CC2)cnc1-c1nc2cc(C(F)(F)C(F)(F)F)nnc2n1C]

Into a 50 mL round-bottom flask were added tert-butyl N-(cyclopropanecarbonyl)-N-[5-ethylsulfonyl-6-[7-methyl-3-(1,1,2,2,2-pentafluoroethyl)imidazo[4,5-c]pyridazin-6-yl]-3-pyridyl]carbamate (1.0 eq, 165 mg, 0.26 mmol), DCM (2 mL) and TFA (2 mL) at room temperature. The resulting mixture was concentrated under vacuum. The residue was purified by reverse flash chromatography with the following conditions: column, C18 silica gel; mobile phase, MeCN in Water (0.1% NH₃·H₂O), 80% gradient in 10 min; detector, UV 254 nm. This resulted in N-[5-ethylsulfonyl-6-[7-methyl-3-(1,1,2,2,2-pentafluoroethyl)imidazo[4,5-c]pyridazin-6-yl]-3-pyridyl]cyclopropanecarboxamide (130 mg, 98%) as a yellow solid.

### Synthesis of 6-[5-(5-cyclopropyltetrazol-1-yl)-3-ethylsulfonyl-2-pyridyl]-7-methyl-3-(1,1,2,2,2-pentafluoroethyl)imidazo[4,5-c]pyridazine

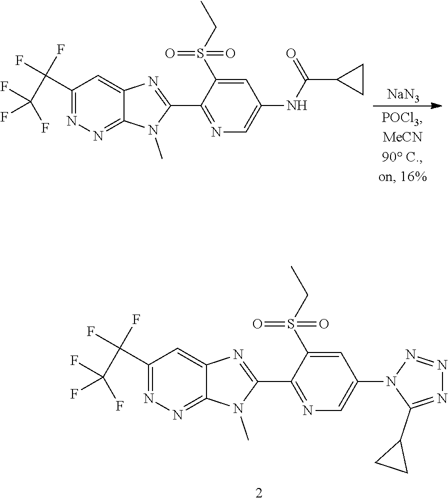 [SMILES: CCS(=O)(=O)c1cc(NC(=O)C2CC2)cnc1-c1nc2cc(C(F)(F)C(F)(F)F)nnc2n1C ; CCS(=O)(=O)c1cc(-n2nnnc2C2CC2)cnc1-c1nc2cc(C(F)(F)C(F)(F)F)nnc2n1C ; "NaN3"]

Into a 8 mL vial were added N-[5-ethylsulfonyl-6-[7-methyl-3-(1,1,2,2,2-pentafluoroethyl)imidazo[4,5-c]pyridazin-6-yl]-3-pyridyl]cyclopropanecarboxamide (1.0 eq, 80 mg, 0.16 mmol), CH₃CN (3 mL), POCl₃ (15 eq, 0.23 mL, 2.4 mmol) and NaN₃ (3.1 eq, 31 mg, 0.48 mmol) at room temperature. The mixture was stirred at 90 degrees C. overnight. The mixture was basified to pH 8 with saturated NaHCO₃ (aq.). The resulting mixture was extracted with CH₂Cl₂ (3×20 mL). The combined organic layers were concentrated under reduced pressure. The residue was purified by reverse flash chromatography with the following conditions: column, C18 silica gel; mobile phase, MeCN in Water (0.1% NH₃·H₂O), 80% gradient in 10 min; detector, UV 254 nm. This resulted in 6-[5-(5-cyclopropyltetrazol-1-yl)-3-ethylsulfonyl-2-pyridyl]-7-methyl-3-(1,1,2,2,2-pentafluoroethyl)imidazo[4,5-c]pyridazine (14 mg, 16%) as a white solid.

### Example 2: Physicochemical Characterization

| Compound | (ES, m/z) | ¹H NMR Spectra |
|---|---|---|
| 1 | 530 [M + H]+ | 1H NMR (400 MHz, CDCl₃, ppm): 9.73 (d, J = 1.6 Hz, 1 H), |
| | | 9.25 (d, J = 1.6 Hz, 1 H), 8.25 (s, 1 H), 4.18 (s, 3 H), 3.90 (q, |
| | | J = 7.2 Hz, 2 H), 2.05 (m, 1 H), 1.55-1.50 (m, 2 H), 1.47-1.43 |
| | | (m, 3 H) |
| 2 | 530 [M + H]+ | 1H NMR (400 MHz, CDCl₃, ppm): 9.43 (d, J = 2.4 Hz, 1 H), |
| | | 8.92 (d, J = 2.4 Hz, 1 H), 8.26 (s, 1 H), 4.18 (s, 3 H), 3.95 (q, |
| | | J = 7.2 Hz, 2 H), 2.05 (m, 1 H), 1.55-1.51 (m, 2 H), 1.48-1.39 |
| | | (m, 3 H) |
| 3 | 484 [M + H]+ | (400 MHz, CDCl3, ppm): δ 9.48 (d, J = 2.0 Hz, 1H), 8.92 (d, |
| | | J = 2.0 Hz, 1H), 7.41 (s, 1H), 5.04 (q, J = 8.4 Hz, 2H), 4.38 |
| | | (s, 3H), 3.99 (s, 3H), 3.92 (q, J = 7.6 Hz, 2H), 1.42 (t, J = 7.2 |
| | | Hz, 3H) |
| 4 | 504 [M + H]+ | (300 MHz, CDCl3, ppm): δ 9.51 (d, J = 2.1 Hz, 1H), 8.95 (d, |
| | | J = 2.1 Hz, 1H), 8.26 (s, 1H), 4.40 (s, 3H), 4.17 (s, 3H), |
| | | 3.97-3.89 (m, 2H), 1.45 (t, J = 7.5 Hz, 3H). |
| 5 | 532 [M + H]⁺ | (300 MHz, CDCl₃, ppm): δ 9.36 (d, J = 2.1 Hz, 1H), 9.07 (d, |
| | | J = 2.1 Hz, 1H), 8.12 (s, 1H), 5.01-4.93 (m, 1H), 4.15 (s, |
| | | 3H), 3.38-3.31 (m, 2H), 1.48-1.44 (m, 9H). |
| 6 | 518 [M + H]⁺ | (300 MHz, CDCl₃, ppm): δ 9.44 (s, 1H), 8.90 (s, 1H), 8.26 |
| | | (s, 1H), 4.66 (q, J = 7.2 Hz, 2H), 4.17 (s, 3H), 3.93 (q, J = |
| | | 7.2 Hz, 2H), 1.76 (t, J = 7.2 Hz, 3H), 1.44 (t, J = 7.2 Hz, |
| | | 3H). |
| 7 | 504 [M + H]+ | 1H NMR (400 MHz, CDCl₃, ppm): 9.75 (d, J = 2.8 Hz, 1 H), |
| | | 9.23 (d, J = 2.8 Hz, 1 H), 8.23 (s, 1 H), 4.52 (s, 3 H), 4.12 (s, |
| | | 3 H), 3.85 (q, J = 10.0 Hz, 2 H), 1.43 (t, J = 10.0 Hz, 3 H) |
| 8 | [M + H] + 459 | (400 MHz, CDCl3, ppm) δ: 9.66 (s, 1H), 9.15 (s, 1H), 7.83 |
| | | (s, 1H), 6.58 (s, 1H), 4.49 (s, 3H), 4.11-4.03 (m, 2H), 3.90 |
| | | (s, 3H), 3.83-3.77 (m, 2H), 1.40-1.35 (t, J = 7.4 Hz, 3H). |
| 9 | 518 [M + H]+ | (300 MHz, CDCl3, ppm): δ 9.77 (d, J = 2.1 Hz, 1H), 9.25 (d, |
| | | J = 2.1 Hz, 1H), 8.71 (s, 1H), 4.53 (s, 3H), 4.19 (s, 3H), 3.84 |
| | | (q, J = 7.5 Hz, 2H), 1.45 (q, J = 7.5 Hz, 3H). |
| 10 | 532 [M + H]+ | (300 MHz, CDCl3, ppm): δ 9.78 (d, J = 1.8 Hz, 1H), 9.24 (d, |
| | | J = 2.1 Hz, 1H), 8.23 (s, 1H), 5.27-5.18 (m, 1H), 4.08 (s, |
| | | 3H), 3.89-3.82 (m, 2H), 1.77 (d, J = 6.9 Hz, 1H), 1.44 (t, J = |
| | | 7.5 Hz, 3H). |
| 11 | 502[M + H]+ | (300 MHz, DMSO-d6, ppm): δ 9.41 (d, J = 1.8 Hz, 1H), |
| | | 8.89 (d, J = 1.8 Hz, 1H), 8.86 (s, 1H), 8.66 (s, 1H), 7.42 (s, |
| | | 1H), 6.72 (t, J = 54 Hz, 1H), 5.28 (t, J = 15 Hz, 2H), 4.54- |
| | | 4.47 (m, 2H), 4.30 (s, 3H), 1.33 (t, J = 7.5 Hz, 3H). |
| 12 | 504 [M + H]+ | 1H NMR (300 MHz, CDCl3, ppm): δ 9.31 (s, 1H), 8.76 (d, J = |
| | | 1.8 Hz, 1H), 8.26 (s, 1H), 4.18 (s, 3H), 3.95 (q, J = 7.5 Hz, |
| | | 2H), 2.87 (s, 3H), 1.46 (t, J = 7.2 Hz, 3H). |
| 13 | 517[M + H]+ | (300 MHz, CDCl3, ppm): δ 9.49 (s, 1H), 9.11 (s, 1H), 8.93 |
| | | (s, 1H), 8.73 (s, 1H), 4.39 (s, 3H), 4.03 (s, 3H), 3.99-3.95 |
| | | (m, 2H), 1.45 (t, J = 7.5 Hz, 3H). |
| 14 | 503[M + H]+ | (400 MHz, CDCl3, ppm): δ 9.72 (d, J = 2.0 Hz, 1H), 9.22 (d, |
| | | J = 2.0 Hz, 1H), 8.73(d, J = 1.6 Hz, 1H), 8.30 (d, J = 1.6 Hz, |
| | | 1H), 4.51 (s, 3H), 3.95-3.90 (m, 5H), 1.42 (t, J = 7.2 Hz, |
| | | 3H). |
| 15 | 503[M + H]+ | (400 MHz, CDCl3, ppm): δ 9.46 (d, J = 2.0 Hz, 1H), 8.92 (d, |
| | | J = 2.0 Hz, 1H), 8.76 (d, J = 1.6 Hz, 1H), 8.33 (d, J = 1.6 Hz, |
| | | 1H), 4.36 (s, 3H), 4.05-3.99 (m, 5H), 1.44 (t, J = 7.2 Hz, |
| | | 3H). |
| 16 | 566[M + H]+ | (400 MHz, CDCl3, ppm): δ 9.35 (d, J = 2.4 Hz, 1H), 8.84 (s, |
| | | 1H), 8.53 (d, J = 2.4 Hz, 1H), 7.76-7.67 (m, 5H), 4.02 (s, |
| | | 3H), 3.74 (q, J = 9.6 Hz, 2H), 1.03 (t, J = 9.6 Hz, 3H). |

### Example 3: Screening Method to Test Activity of Compounds of Formula (I), (Ia) or (Ib) Against Larvae of Aedes aegypti

Eight A. aegypti L1 larvae in a total volume of 64 μL water are added to 384-well microplates containing compounds of formula (I), (Ia) and (Ib) formulated in 100% DMSO. The plates are incubated for 48 h at 27° C. Individual well images are acquired and analyzed using a pipeline to quantitatively assess the amount of larvae biomass accumulating at the bottom of the well. The efficacy of a compound at a given dose is expressed as “percentage of mortality” and determined by comparison to average biomass descriptors of positive and negative control wells containing 1.0 μM Fipronil or DMSO only, respectively. Dose response assay are conducted to determine an EC₅₀ value.

### Example 4: Screening Method to Test Contact Activity of Compounds of Formula (I), (Ia) or (Ib) Against Adult Ctenocephalides felis

Compounds of formula (I), (Ia) and (Ib) dissolved in 100% DMSO are diluted in acetone to a desired concentration. The resulting formulation is used to coat the fibers of a 0.5-inch-long piece of pipe cleaner placed in a glass scintillation vial. The vial is capped with a rubber gasket and filter paper insert. After the acetone has evaporated, ten adult Ctenocephalides felis fleas are added to each vial. The vials are then incubated at 22° C., 80% relative humidity, and 12 hours of light/12 hours of dark until a visual evaluation for mortality is performed at 72 hours post-treatment. Compound efficacy at a given dose is expressed as percentage mortality and adjusted to remove background mortality observed in control vials containing DMSO only. A dose response series of treated vials is implemented to determine EC₅₀ values. Compound 14 exhibits EC₅₀ values of between 100 μM and 10 μM. Compound 7 exhibits EC₅₀ values of less than 10 μM.

### Example 5: Screening Method to Test Contact Activity of Compounds of Formula (I), (Ia) or (Ib) Against Adult Rhipicephalus Sanguineus

Compounds of formula (I), (Ia) and (Tb) formulated in 100% DMSO are diluted in a solution containing acetone and Triton X-100 (0.02%). The resulting formulation is used to coat the inner walls of glass scintillation vials and the filter paper covering the cap of the vial. Once dried, ten adult R. sanguineus ticks are added to the vials. The vials are incubated at 24° C., 95% relative humidity, and 12 hours of light/12 hours of dark until evaluation. Ticks are visually evaluated for mortality at 48 hours post-treatment. Compound efficacy at a given dose is expressed as percentage mortality. A dose response series is implemented to determine EC₅₀ values. Compounds 1, 7, 14 exhibit EC₅₀ values of between 100 μM and 10 μM. Compound 10 exhibits EC₅₀ values of less than 10 μM.

### Example 6: Screening Method to Test Ingestion Activity of Compounds of Formula (I), (Ia) or (Ib) Against Adult Ctenocephalides felis

Compounds of formula (I), (Ia) and (Ib) dissolved in 100% DMSO are added to bovine blood and offered via an artificial membrane feeding system to adult C. felis fleas. The motility of fleas is then visually inspected 48 h post treatment. Efficacy is expressed in % motility reduction compared to controls containing blood treated with DMSO only. A dose response series is implemented to determine EC₅₀ values. Compounds 2, 7, 8, 12, 13 exhibit ECso values of between 100 μM and 10 μM. Compounds 1, 4, 6, 9, 14, 15 exhibit EC₅₀ values of less than 10 μM.

### Example 7: Screening Method to Test Ingestion Activity of Compounds of Formula (I), (Ia) or (Ib) Against Adult Ctenocephalides felis (Alternative Method)

Compounds of formula (I), (Ia) and (Ib) dissolved in 100% DMSO are diluted in bovine blood with sodium heparin anti-coagulant. The resulting formulation is offered via an artificial membrane feeding system to approximately 15 adult C. felis fleas previously dispensed into 24-well plates. The plates are then incubated in a double-chambered device. Visual evaluation for mortality is performed at 48 hours post-treatment. Compound efficacy at a given dose is expressed as percentage mortality and determined by normalization to positive and negative controls. A dose response series is implemented to determine EC₅₀ values.

### Example 8: Screening Method for the Determination of Aqueous Solubility of Compounds of Formula (I), (Ia) or (Ib) from DMSO Stock Solution Using LC-UV

The aqueous solubility of the compounds of formula (I), (Ia) and (Ib) is determined by comparing the amount dissolved in buffer to the amount in an acetonitrile/water (1/1) solution. Starting from a 10 mM DMSO stock solution aliquots are diluted with acetonitril/water (1/1) or buffer, respectively. After 24 h of shaking, the solutions are filtrated and analyzed by LC-UV. The amount dissolved in buffer is compared to the amount in the acetonitrile/water solution. Solubility is usually measured from 0.001 to 0.125 mg/ml at a DMSO concentration of 2.5%. If more than 90% of a test compound is dissolved in buffer, the value is marked with “>”.

### Example 9: Determination of Metabolic Stability with Rat Liver Microsomes In Vitro

The metabolic degradation of the compounds of formula (I), (Ia) and (Tb) is assayed with liver microsomes from male rats. The final incubation contains suitable buffer for pH 7.6 (0.1 M), suitable salts (5 mM), microsomal protein (0.5 mg/ml) and the test compound at a final concentration of 1 μM. After incubation at suitable temperature, the reactions are initiated by addition of NADPH (1 mM) and terminated by transfering an aliquot into solvent after different time points. Additionally, the NADPH-independent degradation is monitored in incubations without NADPH, terminated at the last time point. The quenched incubations are pelleted by centrifugation. An aliquot of the supernatant is assayed by LC-MS/MS for the amount of parent compound. The half-life is determined by the slope of the semilogarithmic plot of the concentration-time profile. The intrinsic clearance is calculated by considering the amount of protein in the incubation.

### Example 10: Determination of Metabolic Stability with Dog Hepatocytes In Vitro

The metabolic degradation of the compounds of formula (I), (Ia) and (Ib) is assayed in a hepatocyte suspension. Hepatocytes are incubated in an appropriate buffer system containing a suitable concentration of species serum. Following a preincubation in an incubator at suitable conditions a sample of test compound solution is added into hepatocyte suspension of suitable amount and concentration. The cells are incubated for several hours and samples are taken at several timepoints. Samples are transferred into acetonitrile and parent compound decline determined from the supernatant by means of HPLC-MS/MS. Clearance is calculated considering initial concentration, cell density and species specific parameters.

### Example 11: Evaluation of Acaracidal Activity of Compounds of Formula (I), (Ia) or (Ib) Administered Orally in Rats Using Dermacentor variabilis

On Day −1, rats are sedated, fitted with an Elizabethan collar, and infested with approximately 35 nymphal stage D. variabilis ticks. On Day 0, each rat is treated via oral gavage with the appropriate formulation of placebo, positive control, or the compound of formula (I), (Ta) and (Tb) at the appropriate dose. On Day 3, all rats are euthanized, and ticks are removed, counted, and disposed. Compound efficacy is determined by means of statistical analysis of the results from treatment groups vs. results from placebo group.

### Example 12: Evaluation of Acaracidal Activity of Compounds of Formula (I), (Ia) or (Ib) Administered Orally in Dogs Using Rhipicephalus sanguineus

Controlled studies are performed for laboratory assessment of efficacy of ticks in dogs by applying unfed adult Rhipicephalus sanguineus ticks directly onto the topline of the dog (infestation). Dogs may or may not be housed in an enclosure, smaller than their usual housing, for a short time after tick infestation, to encourage tick attachment. Typical unfed tick numbers used for infestation in efficacy assessment is 50. A live tick retention rate of at least 20% on non-treated animals is generally accepted for experimental laboratory infestations.

For measurement of efficacy of the compounds of formula (I), (Ta) and (Ib) on existing tick infestations, ticks are placed on dogs 48 hours prior to treatment and removed and counted 48 hours after treatment. For measurement of the compounds of formula (I), (Ta) and (Ib) on new tick infestations after treatment, ticks are placed on dogs with removal and counting 48 hours after infestation.

### Example 13: Insecticidal Test on Spodoptera litura

The compounds of formula (I), (Ta) and (Tb) of the present invention or salts thereof are separately dispersed in water and diluted to 50 ppm. Cabbage leaves are dipped in the dispersion for about 30 seconds. After air dried, the leaves are placed in a plastic Petri dish with a diameter of 9 cm and inoculated with ten 2nd-instar larvae of Spodoptera litura, after which the dish is closed and then allowed to stand in a room at 25° C. 8 days after the inoculation, the numbers of the dead and alive insects are counted. The corrected mortality rate is calculated according to the following formula and the insecticidal activity is evaluated according to the following criteria.

$$Corrected mortality rate ( \% ) = 100 \times ( Survival rate in an untreated plot - Survival rate in a treated plot ) / Survival rate in an untreated plot$$

A: the corrected mortality rate is 100%
B: the corrected mortality rate is 90 to 99%
C: the corrected mortality rate is 80 to 89%
D: the corrected mortality rate is 50 to 79%

As a result, compounds 9, 14 show the activity level evaluated as A.

### Example 14: Insecticidal Test on Plutella xylostella

The compounds of formula (I), (Ia) and (Ib) of the present invention or salts thereof are separately dispersed in water and diluted to 50 ppm. Ten adults of Plutella xylostella are released onto Chinese cabbage seedlings and allowed to lay eggs thereon. 2 days after the release of the adults, the seedling is dipped in the dispersion for about 30 seconds. After air dried, the seedling is kept in a room at 25° C. 6 days after the dip treatment, the numbers of the dead and alive insects are counted. The corrected mortality rate is calculated according to the following formula and the insecticidal activity was evaluated according to the following criteria.

$$Corrected mortality rate ( \% ) = 100 \times ( Survival rate in an untreated plot - Survival rate in a treated plot ) / Survival rate in an untreated plot$$

A: the corrected mortality rate is 100%
B: the corrected mortality rate is 90 to 99%
C: the corrected mortality rate is 80 to 89%
D: the corrected mortality rate is 50 to 79%

As a result, the compounds 1, 9, 10, 14, 15 and 16 show the activity level evaluated as A.

### Example 15: Insecticidal Test on Laodelphax striatellus

The compounds of formula (I), (Ta) and (Tb) of the present invention or salts thereof are separately dispersed in water and diluted to 50 ppm. Rice plant seedlings are dipped in the dispersion for about 30 seconds. After air dried, the seedlings are put into a separate glass test tube and inoculated with ten 3rd-instar nymphs of Laodelphax striatellus and then the glass test tube is kept in a room at 25° C. 8 days after the inoculation, the numbers of the dead and alive insects are counted. The corrected mortality rate is calculated according to the following formula and the insecticidal activity is evaluated according to the following criteria.

$$Corrected mortality rate ( \% ) = 100 \times ( Survival rate in an untreated plot - Survival rate in a treated plot ) / Survival rate in an untreated plot$$

A: the corrected mortality rate is 100%
B: the corrected mortality rate is 90 to 99%
C: the corrected mortality rate is 80 to 89%
D: the corrected mortality rate is 50 to 79%

As a result, the compounds 1, 4, 9 and 10 showed the activity level evaluated as A.

### Example 16: Insecticidal Test on Myzus Persicae

The compounds of formula (I), (Ia) and (Ib) of the present invention or salts thereof are separately dispersed in water and diluted to 50 ppm. Chinese cabbage plants are planted in plastic pots (diameter: 8 cm, height: 8 cm). Aphids (Myzus persicae) are propagated on the plants and then the number of surviving aphids is counted. The dispersion is applied to the foliage of the potted plants. After the plants are air dried, these are kept in a greenhouse. 6 days after the application, the number of the alive insects is counted on the plants. The control rate is calculated according to the following formula and the control efficacy is evaluated according to the following criteria.

$$Control rate ( \% ) = 100 - \{ ( T \times Ca ) / ( Ta \times C ) \} \times 100$$

Ta: the number of alive insects before the foliar application in a treated plot
T: the number of alive insects after the foliar application in a treated plot
Ca: the number of alive insects before the foliar application in an untreated plot
C: the number of alive insects after the foliar application in an untreated plot
A: the control rate is 100%
B: the control rate is 90 to 99%
C: the control rate is 80 to 89%
D: the control rate is 50 to 79%

As a result, the compounds 1, 4, 9, 10, 14 and 15 show the control efficacy level evaluated as A.

### REFERENCES

The following references, to the extent that they provide exemplary procedural or other details supplementary to those set forth herein, are specifically incorporated herein by reference.
(1) WO 2016/030229
(2) WO 2016/059145
(3) WO 2016/071214
(4) WO 2018/095795
(5) WO 2020/182577

The following clauses are also part of the general disclosure and are comprised by the spirit and scope o the present invention:

1. A compound of formula (I) or formula (Ia) or formula (Ib)

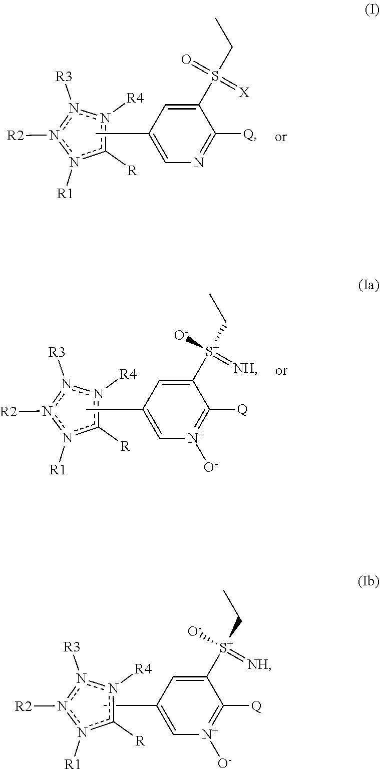

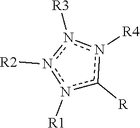

is attached to the central pyridine ring via the C atom or any N atom of the tetrazole moiety; preferably via the C atom;
R is independently absent, hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈- cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkoxy, such as methoxy, ethoxy, propoxy, C₁-C₆-haloalkoxy, such as O—CF₃, C₁-C₆-alkyl-C₁-C₆-alkoxy, such as CH₂—O—CH₃;
R1, R2, R3, R4 are each independently absent, hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃;
Q is selected from the group consisting of: Q1, Q2, Q3, wherein:
Q1 is an unsubstituted or substituted monocyclic, preferably 5- or 6-membered, heteroaryl or heterocyclyl containing one, two or three heteroatoms, which are each independently selected from the group consisting of N, O, S, S(O), S(O)₂; wherein preferably Q1 if substituted is substituted with one, two, three or four substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂—C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, ═O, S(O)NRₓR_{y}, such as S(O)NH—CF₃;
Q2 is an unsubstituted or substituted fused bicyclic, preferably 9- or 10-membered, heteroaryl or heterocyclyl containing one, two, three, four or five heteroatoms, which are each independently selected from the group consisting of N, O, S, S(O), S(O)₂; wherein preferably Q2 if substituted is substituted with one, two, three, four, five or six substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH, C₁-C₆-haloalkyl, such as CF₃, C_{2F}s, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂—C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, S(O)ᵣ—(C₁-C₆-alkyl), such as S-methyl; S(O)-methyl, S(O)₂-methyl; S(O)ₛ—(C₁-C₆-haloalkyl), such as S—CF₃, S(O)—CF₃, S(O)₂—CF₃, ═O, S(O)NRₓR_{y}, such as S(O)NH—CF₃;
Q3 is an unsubstituted or substituted fused tricyclic, preferably 12- or 13-membered, heteroaryl or heterocyclyl containing one, two, three, four, five, six or seven heteroatoms, which are each independently selected from the group consisting of N, O, S, S(O), S(O)₂; wherein preferably Q3 if substituted is substituted with one, two, three, four, five, six, seven or eight substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂—C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, S(O)ᵣ—(C₁-C₆-alkyl), such as S-methyl; S(O)-methyl, S(O)₂-methyl; S(O)ₛ—(C₁-C₆-haloalkyl), such as S—CF₃, S(O)—CF₃, S(O)₂—CF₃, ═O, S(O)NRₓR_{y}, such as S(O)NH—CF₃;
Rₓ, R_{y} in each occurrence are independently selected from the group consisting of: hydrogen, halogen, such as F, Cl, Br, I, C₁-C₆-alkyl, such as methyl, ethyl, C₁-C₆-haloalkyl, such as CF₃;
r, s in each occurrence independently are 0, 1 or 2;
the dashed bond ( [symbol] ) signifies a single or double bond;
optionally, wherein at least one nitrogen atom is an N-oxide,
optionally, but preferably in case at least one nitrogen atom is an N-oxide, with the first proviso that if X is present and is NH, the NH nitrogen atom is not an N-oxide;
optionally, but preferably in case at least one nitrogen atom is an N-oxide, with the second proviso that, if possible, the at least one nitrogen atom being an N-oxide is not present in any of the radicals R, R1, R2, R3, R4;
optionally, but preferably in case at least one nitrogen atom is an N-oxide, with the third proviso that, if possible, and if any of Q1, Q2, Q3 is substituted, the at least one nitrogen atom being an N-oxide is not present in any potential substituents of any of Q1, Q2, Q3;
optionally, but preferably in case at least one nitrogen atom is an N-oxide, the at least one nitrogen atom being an N-oxide comprises at least one nitrogen atom within a six-membered heterocyclic moiety containing one or more nitrogen atoms of any one of Q1, Q2, Q3;
or a salt thereof.

2. The compound according to clause 1, wherein Q1, Q2, Q3 are independently selected from the group consisting of:

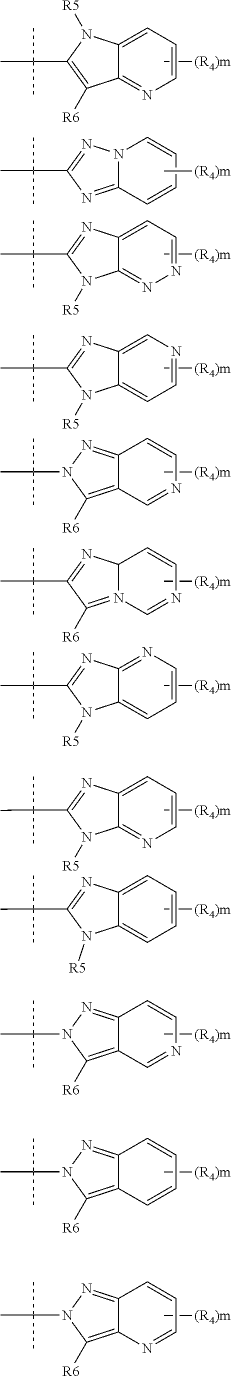

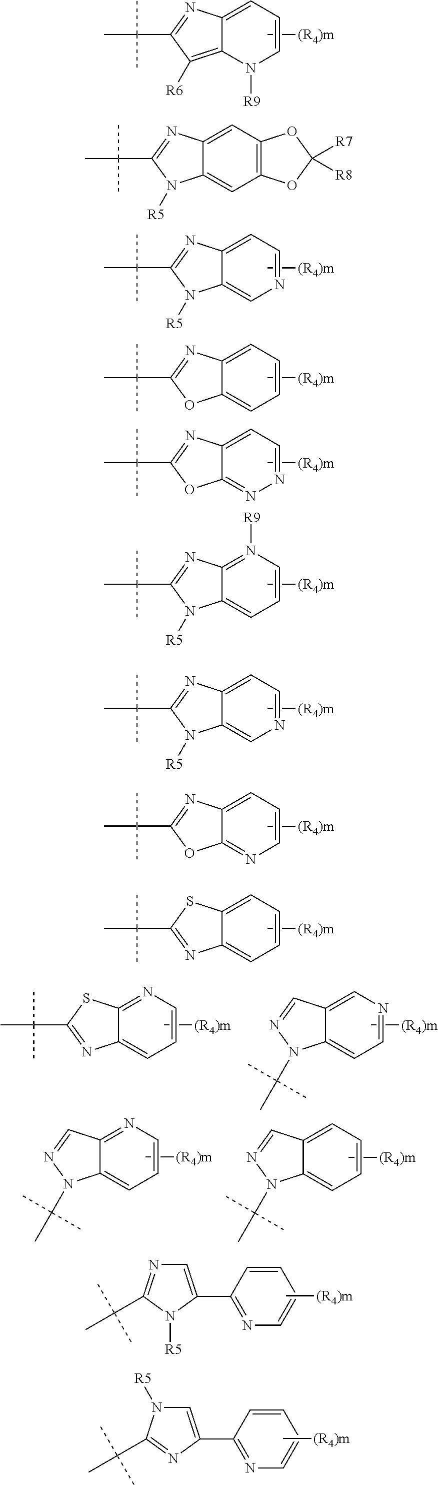

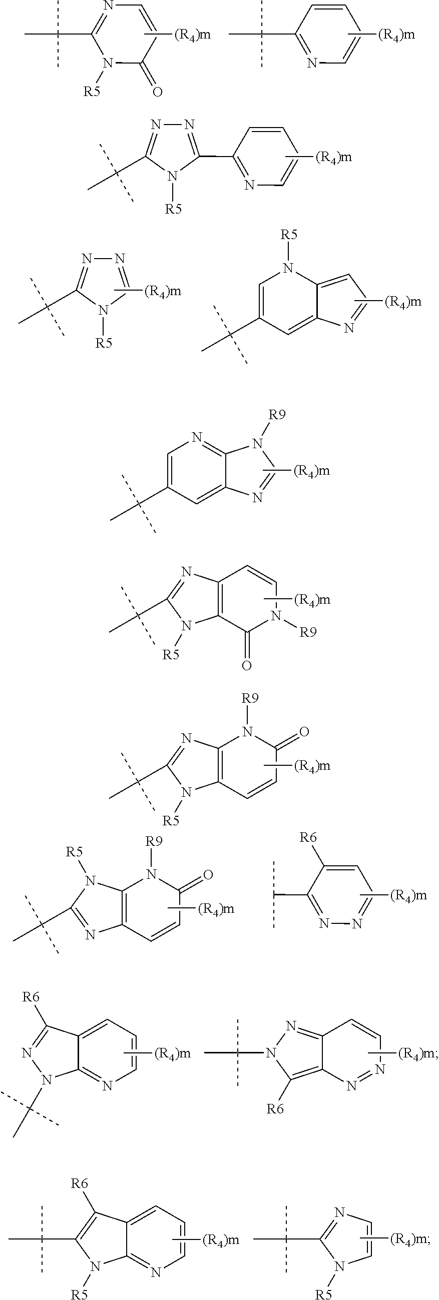

wherein:
R₄ is C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom;
R5, R9 are each independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃;
R6, R7, R8 are each independently hydrogen, halogen, such as F, Cl, Br, I;
m is 0, 1 or 2;
p, q in each occurrence independently are 0, 1 or 2;
or a salt thereof.

3. The compound according to any one of clauses 1 to 2, wherein

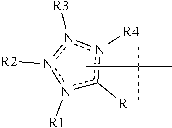

is independently selected from the group consisting of:

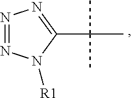

wherein R1 is as defined as in any one of the preceding clauses, but is present, such as R1 is “cyclopropyl”, “methyl”, “ethyl”, “isopropyl”, “phenyl”, “cyclopropyl substituted with CN, OH and/or F”, “CH₂—O—CH₃”, “CF₃”, “CHF₂”, “pyridinyl”, “3-pyridinyl”; and R, R2, R3, R4 are all absent;

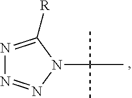

wherein R is as defined as in any one of the preceding clauses, but is present, such as R is “cyclopropyl”. “methyl”, “ethyl”, “isopropyl”, “phenyl”, “cyclopropyl substituted with CN, OH and/or F”, “CH₂—O—CH₃”, “CF₃”, “CHF₂”, “pyridinyl”, “3-pyridinyl”; and R1, R2, R3, R4 are all absent;

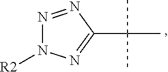

wherein R2 is as defined as in any one of the preceding claims, but is present, such as R2 is “cyclopropyl”. “methyl”, “ethyl”, “isopropyl”, “phenyl”, “cyclopropyl substituted with CN, OH and/or F”, “CH₂—O—CH₃”, “CF₃”, “CHF₂”, “pyridinyl”, “3-pyridinyl”; and R, R1, R3, R4 are all absent;
or a salt thereof.

4. The compound according to any one of clauses 1 to 3, wherein Q1, Q2, Q3 independently are selected from

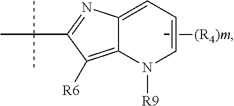

wherein R₄, R6, R9, m are as defined as in any one of the preceding clauses, such as R6 is “hydrogen” or “F”, R9 is “CHF₂” or “CH₂—CHF₂” or “ethyl” or “CH₂-cyclopropyl”, m is 1 and R₄ is “CF₃” or “CHF₂”;

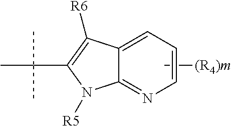

wherein R₄, R5, R6, m are as defined as in any one of the preceding clauses, such as R5 is “hydrogen” or “methyl”, m is 1 and R₄ is “CF₂CF₃” or “CHF₂” and R6 is “hydrogen”;

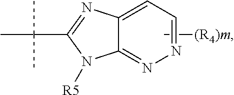

wherein R₄, R5, m are as defined as in any one of the preceding clauses, such as R5 is “methyl”, m is 1 and R₄ is “CHF₂” or “CF₂-cyclopropyl” or “CF₂—CF₃” or “CF₂—CH₂—CH₃” or “CF₃” or “S—CF₃” or “S(O)₂—CF₃” or “O—CH₂—CF₃”;

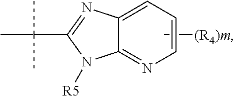

wherein R₄, R5, m are as defined as in any one of the preceding clauses, such as R5 is “methyl”, m is 1 and R₄ is “CHF₂” or “CF₂—CF₃” or “CF₂-cyclopropyl” or “CF₂—CH₂—CH₃” or “S(O)₂—CF₃”;

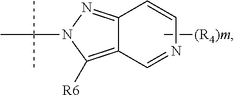

wherein R₄, R6, m are as defined as in any one of the preceding clauses, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃”;

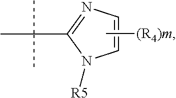

wherein R₄, R5, m are as defined as in any one of the preceding clauses, such as R5 is “methyl”, m is 1 and R₄ is “C(O)NH—CH₂—CF₃”;
or a salt thereof.

5. The compound according to any one of clauses 1 to 3, wherein Q1, Q2, Q3 independently are selected from

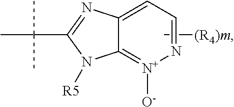

wherein R₄, R5, m are as defined as in any one of the preceding clauses, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃”;

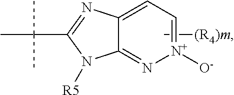

wherein R₄, R5, m are as defined as in any one of the preceding clauses, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃”;

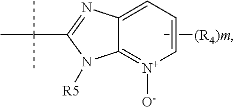

wherein R₄, R5, m are as defined as in any one of the preceding clauses, such as R5 is “methyl”, m is 1 and R₄ is “CHF₂” or “CF₂—CF₃” or “CF₂-cyclopropyl” or “CF₂—CH₂—CH₃” or “S(O)₂—CF₃”;

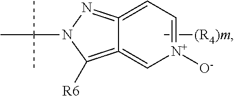

wherein R₄, R6, m are as defined as in any one of the preceding clauses, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃”; or a salt thereof.

6. The compound according to any one of clauses 1 to 5, wherein optionally, if any, the at least one nitrogen atom being an N-oxide comprises the one of the central pyridine moiety selected from the group consisting of formula (II) or formula (IIa) or formula (IIb):

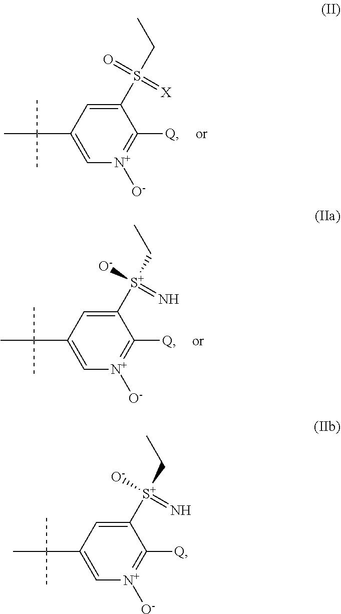

or a salt thereof, wherein X, if present, and Q are as defined as in any one of the preceding clauses and formulae (II), (IIa) and (IIb) are only part of the corresponding formulae (I), (Ia) and (Ib), respectively.

7. A compound of formula (I), (Ia) or (Ib) according to any one of clauses 1 to 6, wherein:

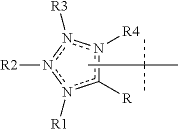

is independently selected from the group consisting of:

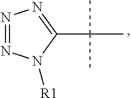

wherein R1 is as defined in any one of the preceding clauses, but is present, such as R1 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R2, R3, R4 are all absent;
or

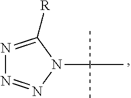

wherein R is as defined in any one of the preceding clauses, but is present, such as R is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R1, R2, R3, R4 are all absent;
or

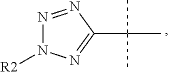

wherein R2 is as defined in any one of the preceding clauses, but is present, such as R2 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R1, R3, R4 are all absent;
and
Q1, Q2, Q3 independently are selected from the group consisting of:

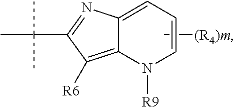

wherein R₄, R6, R9, m are as defined in any one of the preceding clauses, such as R6 is “hydrogen” or “F”, R9 is “CHF₂” or “CH₂—CHF₂” or “ethyl” or “CH₂-cyclopropyl”, m is 1 and R₄ is “CF₃” or “CHF₂”;
or

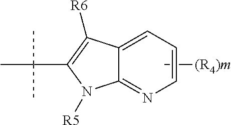

wherein R₄, R5, R6, m are as defined in any one of the preceding clauses, such as R5 is “hydrogen” or “methyl”, m is 1 and R₄ is “CF₂CF₃” or “CHF₂” and R6 is “hydrogen”;
or

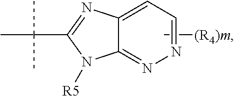

wherein R₄, R5, m are as defined in any one of the preceding clauses, such as R5 is “methyl”, m is 1 and R₄ is “CHF₂” or “CF₂-cyclopropyl” or “CF₂—CF₃” or “CF₂—CH₂—CH₃” or “CF₃” or “S—CF₃” or “S(O)₂—CF₃” or “O—CH₂—CF₃”;
or

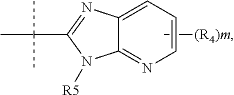

wherein R₄, R5, m are as defined in any one of the preceding clauses, such as R5 is “methyl”, m is 1 and R₄ is “CHF₂” or “CF₂—CF₃” or “CF₂-cyclopropyl” or “CF₂—CH₂—CH₃” or “S(O)₂—CF₃”;
or

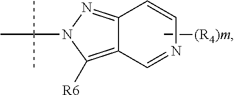

wherein R₄, R6, m are as defined in any one of the preceding clauses, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃”;
or

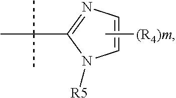

wherein R₄, R5, m are as defined in any one of the preceding clauses, such as R5 is “methyl”, m is 1 and R₄ is “C(O)NH—CH₂—CF₃”;
or

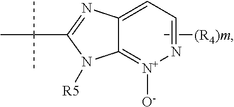

wherein R₄, R5, m are as defined in any one of the preceding clauses, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃”;
or

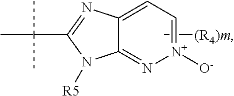

wherein R₄, R5, m are as defined in any one of the preceding clauses, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃”;
or

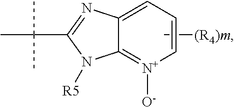

wherein R₄, R5, m are as defined in any one of the preceding clauses, such as R5 is “methyl”, m is 1 and R₄ is “CHF₂” or “CF₂—CF₃” or “CF₂-cyclopropyl” or “CF₂—CH₂—CH₃” or “S(O)₂—CF₃”;
or

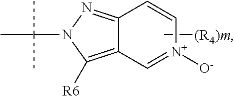

wherein R₄, R6, m are as defined in any one of the preceding clauses, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃”;
and/or
wherein optionally, if any, the at least one nitrogen atom being an N-oxide comprises the one of the central pyridine moiety selected from the group consisting of formula (II) or formula (IIa) or formula (JIb):

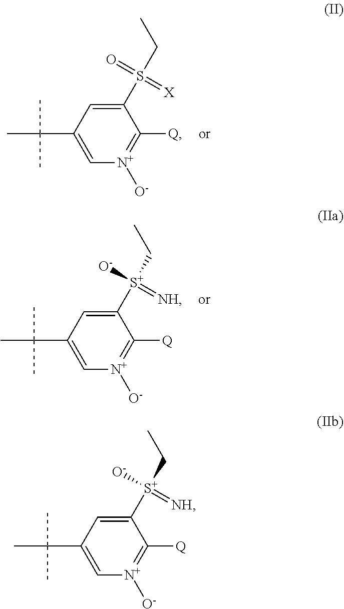

wherein X, if present, and Q are as defined in any one of the preceding clauses and formulae (II), (IIa) and (IIb) are only part of the corresponding formulae (I), (Ia) and (Ib), respectively;
or a salt thereof.

8. The compound according to any one of clauses 1 to 7 selected from the group consisting of:

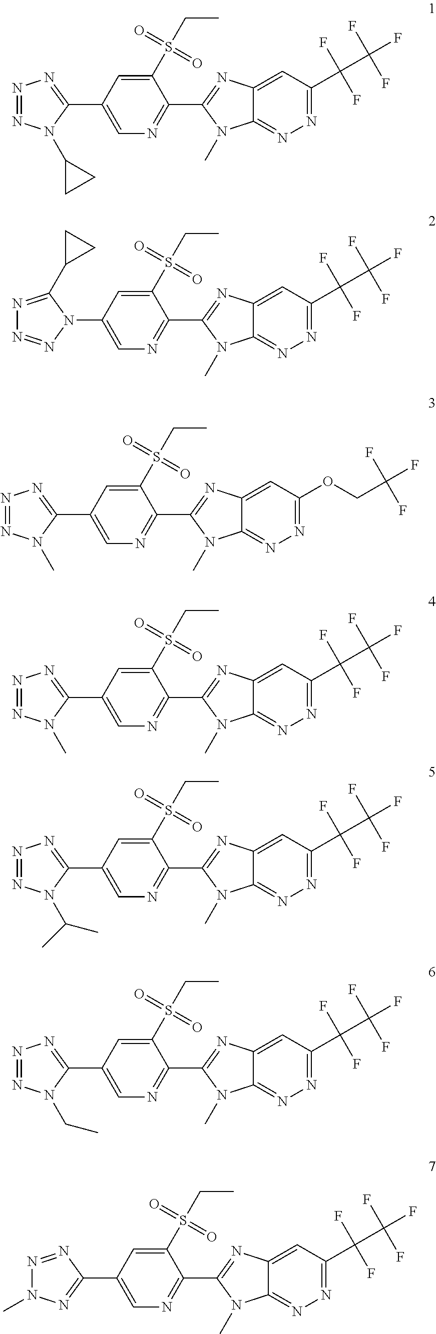

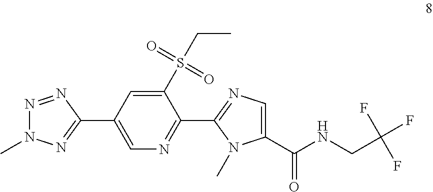 [SMILES: CCS(=O)(=O)c1cc(-c2nnn(C)n2)cnc1-c1ncc(C(=O)NCC(F)(F)F)n1C]

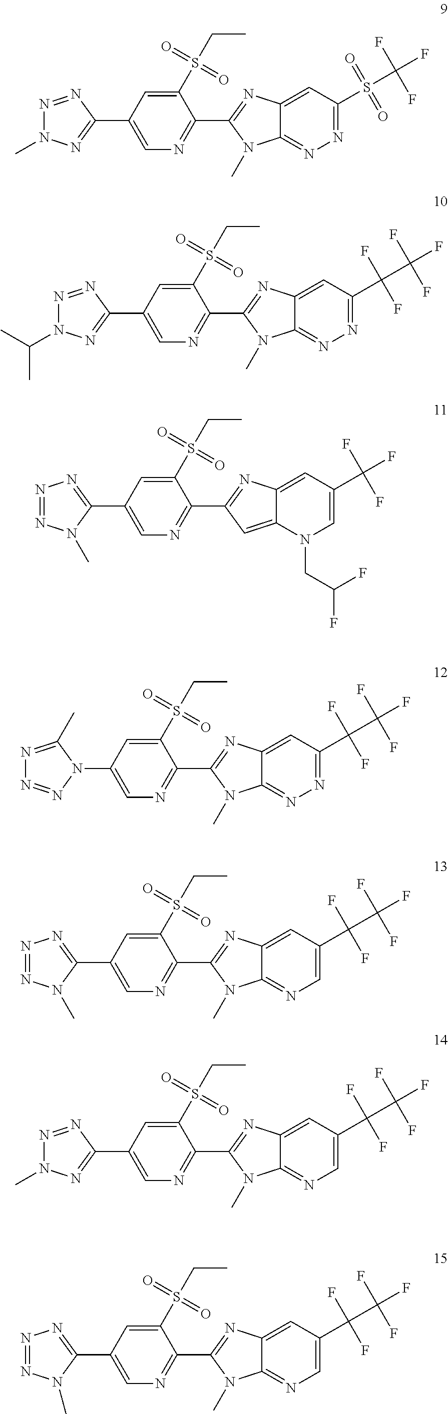

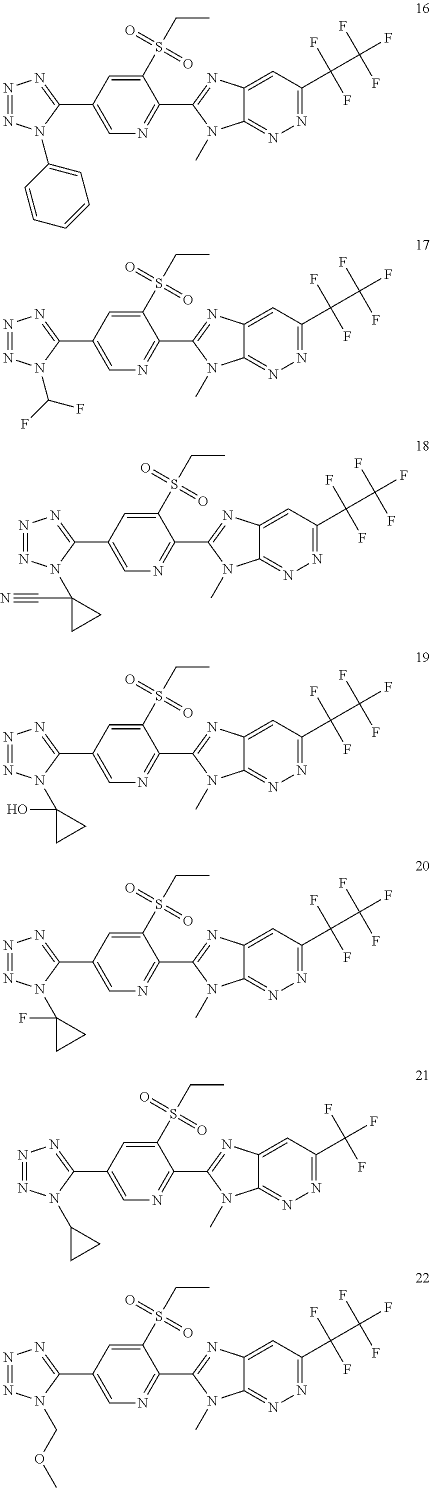

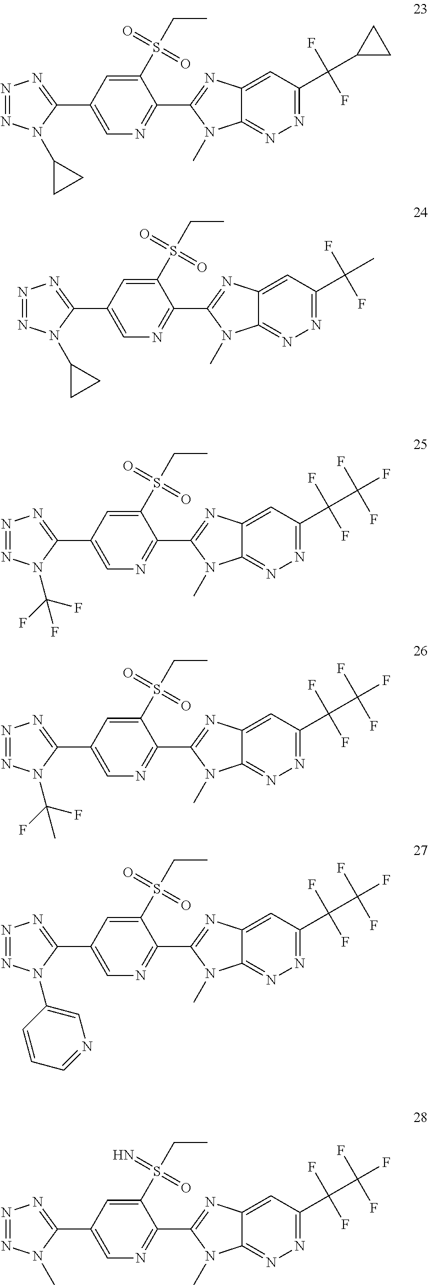 [SMILES: CCS(=O)(=O)c1cc(-c2nnnn2C2CC2)cnc1-c1nc2cc(C(F)(F)C3CC3)nnc2n1C ; CCS(=O)(=O)c1cc(-c2nnnn2C2CC2)cnc1-c1nc2cc(C(C)(F)F)nnc2n1C ; CCS(=O)(=O)c1cc(-c2nnnn2C(F)(F)F)cnc1-c1nc2cc(C(F)(F)C(F)(F)F)nnc2n1C ; CCS(=O)(=O)c1cc(-c2nnnn2C(C)(F)F)cnc1-c1nc2cc(C(F)(F)C(F)(F)F)nnc2n1C ; CCS(=O)(=O)c1cc(-c2nnnn2-c2cccnc2)cnc1-c1nc2cc(C(F)(F)C(F)(F)F)nnc2n1C ; CCS(=N)(=O)c1cc(-c2nnnn2C)cnc1-c1nc2cc(C(F)(F)C(F)(F)F)nnc2n1C]

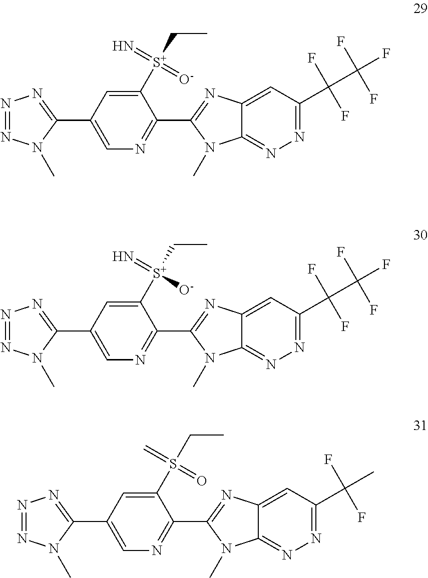

or a pharmaceutically acceptable salt thereof.

9. A pharmaceutical composition comprising one or more compound(s) of formula (I), (Ta) and/or (Tb) according to any one of clauses 1 to 8 or a pharmaceutically acceptable salt thereof and one or more pharmaceutically acceptable excipient(s).

10. A pharmaceutical composition comprising one or more compound(s) of formula (I), (Ta) and/or (Tb) according to any one of clauses 1 to 8 or a pharmaceutically acceptable salt thereof, one or more additional pharmaceutically active agent(s), and one or more pharmaceutically acceptable excipient(s).

11. A compound of formula (I), (Ta) or (Tb) according to any one of clauses 1 to 8 or a pharmaceutically acceptable salt thereof or a pharmaceutical composition according to any one of clauses 9 to 10 for use as a medicament, preferably for use as an antiparasitic medicament.

12. The compound of formula (I), (Ia) or (Ib) according to any one of clauses 1 to 8 or a pharmaceutically acceptable salt thereof or a pharmaceutical composition according to any one of clauses 9 to 10 for use in a method of treatment, prevention and/or control of a parasitic infection and/or infestation in/on an animal, preferably of an ectoparasitic infestation on an animal, more preferably of an infestation of fleas and/or ticks on an animal.

1. A compound of formula (I) or formula (Ia) or formula (Ib) 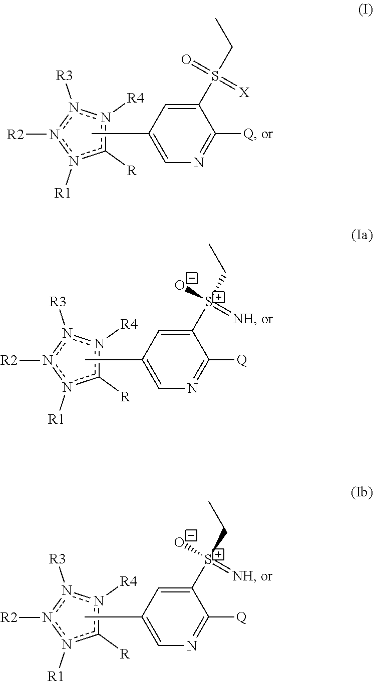
wherein:
X, if present, is independently O or NH; 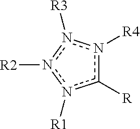
is attached to the central pyridine ring via the C atom or any N atom of the four nitrogen atoms containing five-membered ring system; or via the C atom;
R is independently absent, hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkoxy, such as methoxy, ethoxy, propoxy, C₁-C₆-haloalkoxy, such as O—CF₃, C₁-C₆-alkyl-C₁-C₆-alkoxy, such as CH₂—O—CH₃;
R1, R2, R3, R4 are each independently absent, hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃;
Q is selected from the group consisting of: Q1, Q2, Q3, wherein:
Q1 is an unsubstituted or substituted monocyclic, such as 5- or 6-membered, heteroaryl or heterocyclyl containing one, two or three heteroatoms, which are each independently selected from the group consisting of N, O, S, S(O), S(O)₂; wherein Q1 if substituted is substituted with one, two, three or four substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH₂, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂-C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, ═O, S(O)NRₓR_{y}, such as S(O)NH—CF₃;
Q2 is an unsubstituted or substituted fused bicyclic, such as 9- or 10-membered, heteroaryl or heterocyclyl containing one, two, three, four or five heteroatoms, which are each independently selected from the group consisting of N, O, S, S(O), S(O)₂; wherein Q2 if substituted is substituted with one, two, three, four, five or six substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH₂, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂-C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, S(O)ᵣ—(C₁-C₆-alkyl), such as S-methyl; S(O)-methyl, S(O)₂-methyl; S(O)ₛ—(C₁-C₆-haloalkyl), such as S—CF₃, S(O)—CF₃, S(O)₂—CF₃, ═O, S(O)NRₓR_{y}, such as S(O)NH—CF₃;
Q3 is an unsubstituted or substituted fused tricyclic, such as 12- or 13-membered, heteroaryl or heterocyclyl containing one, two, three, four, five, six or seven heteroatoms, which are each independently selected from the group consisting of N, O, S, S(O), S(O)₂; wherein Q3 if substituted is substituted with one, two, three, four, five, six, seven or eight substituents each independently selected from the group consisting of: halogen, such as F, Cl, Br, I, C₁-C₆-alkyl, such as methyl, ethyl, isopropyl, C₂-C₆-alkenyl, such as ethenyl, CH₂—CH═CH₂, C₁-C₆-haloalkyl, such as CF₃, C₂F₅, CH₂—CHF₂, CH₂—CF₃, CHF₂, CF₂—CH₃, C₂-C₆-haloalkenyl, such as CH═CF—CF₃, C₁-C₆-haloalkoxy, such as O—CH₂—CF₃, O—CH₂-C₂F₅, C₃-C₈-cycloalkyl, such as cyclopropyl, C₃-C₈-cycloalkyl-(C₁-C₆-alkyl), such as cyclopropyl-methyl, C₃-C₈-cycloalkyl substituted with one or more halogens, such as cyclopropyl substituted with one or more F, C₃-C₈-cycloalkyl-(C₁-C₆-haloalkyl), such as cyclopropyl-CF₂, C(O)—(C₁-C₆-alkyl), such as C(O)-methyl, heterocyclyl, such as aziridinyl, oxetanyl, aryl, such as phenyl, heteroaryl, such as pyridine or pyridine substituted with one or more C₁-C₆-haloalkyl or C₁-C₆-haloalkoxy, such as CF₃, CH₂—CF₂—CF₃, or O—CH₂—CF₂—CF₃, S(O)ᵣ—(C₁-C₆-alkyl), such as S-methyl; S(O)-methyl, S(O)₂-methyl; S(O)ₛ—(C₁-C₆-haloalkyl), such as S—CF₃, S(O)—CF₃, S(O)₂—CF₃, ═O, S(O)NRₓR_{y}, such as S(O)NH—CF₃;
Rₓ, R_{y} in each occurrence are independently selected from the group consisting of: hydrogen, halogen, such as F, Cl, Br, I, C₁-C₆-alkyl, such as methyl, ethyl, C₁-C₆-haloalkyl, such as CF₃;
r, s in each occurrence independently are 0, 1 or 2;
the dashed bond ( [symbol] ) signifies a single or double bond;
optionally, wherein at least one nitrogen atom is an N-oxide,
optionally, in case at least one nitrogen atom is an N-oxide, with the first proviso that no nitrogen atom in the four nitrogen atoms containing five-membered ring system is an N-oxide;
optionally, in case at least one nitrogen atom is an N-oxide, with the second proviso that if X is present and is NH, the NH nitrogen atom is not an N-oxide;
optionally, in case at least one nitrogen atom is an N-oxide, with the third proviso that, if possible, the at least one nitrogen atom being an N-oxide is not present in any of the radicals R, R1, R2, R3, R4;
optionally, in case at least one nitrogen atom is an N-oxide, with the fourth proviso that, if possible, and if any of Q being Q1, Q2 or Q3 is substituted, the at least one nitrogen atom being an N-oxide is not present in any potential substituents of any of Q being Q1, Q2 or Q3;
optionally, in case at least one nitrogen atom is an N-oxide, the at least one nitrogen atom being an N-oxide comprises at least one nitrogen atom within a six-membered heterocyclic moiety containing one or more nitrogen atoms of any one of Q being Q1, Q2 or Q3;
or a salt thereof.

2. The compound according to claim 1, wherein Q is Q1, Q2 or Q3, which are independently selected from the group consisting of: 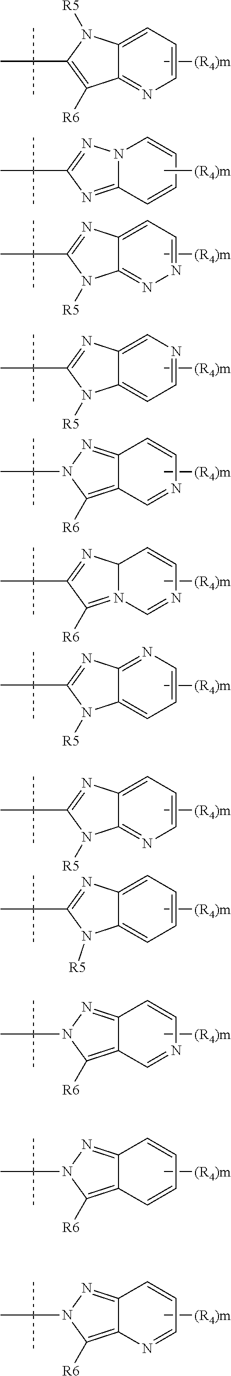 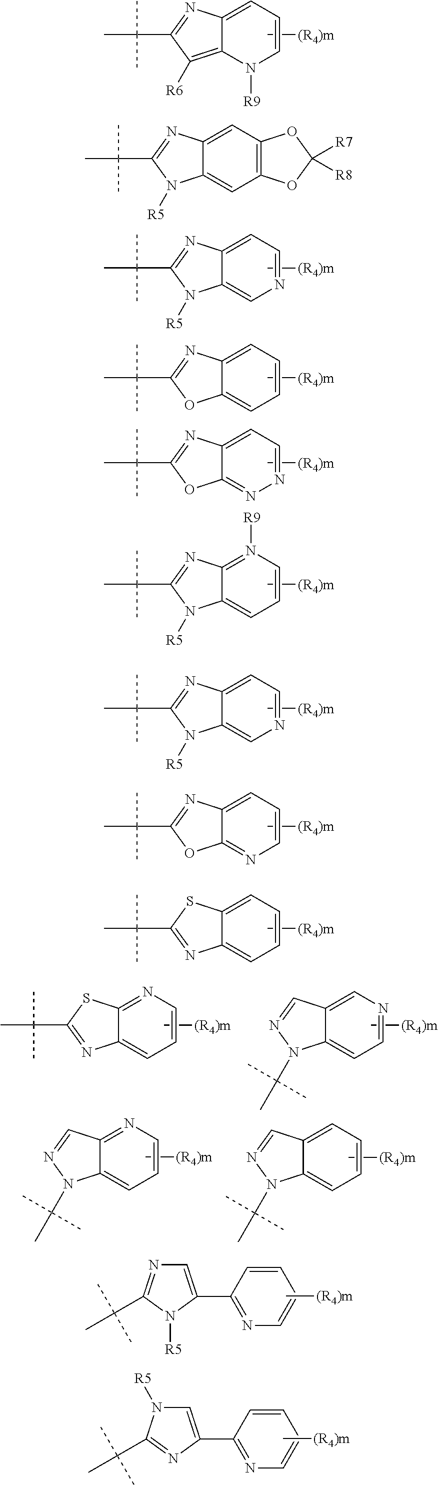 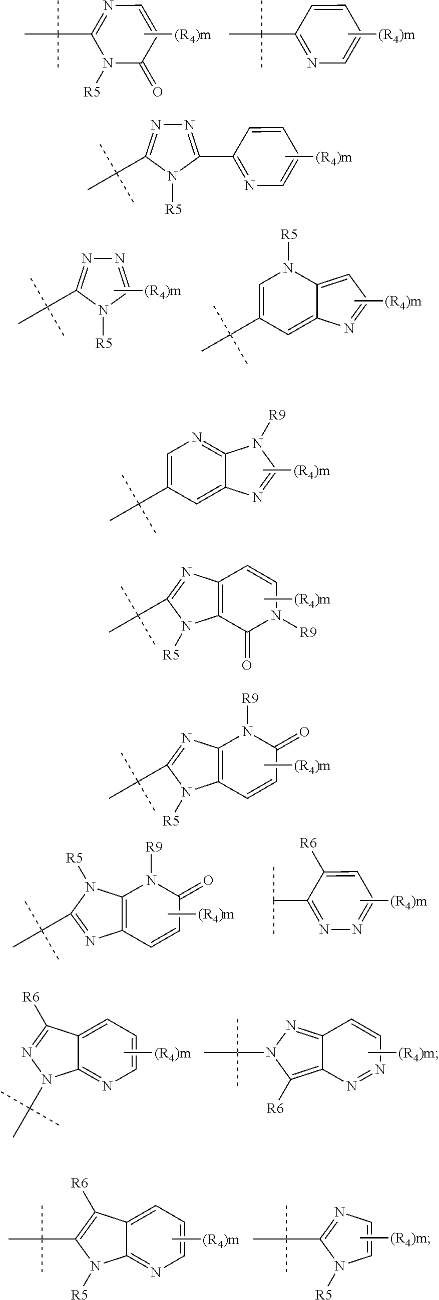
wherein:
R₄ is C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom;
R5, R9 are each independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃;
R6, R7, R8 are each independently hydrogen, halogen, such as F, Cl, Br, I;
m is 0, 1 or 2;
p, q in each occurrence independently are 0, 1 or 2;
or a salt thereof.

3. The compound according to claim 1,
wherein 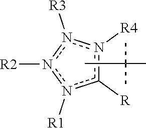
is independently selected from the group consisting of: 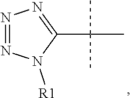
wherein R1 is as defined as in claim 1, but is present, such as R1 is “cyclopropyl”, “methyl”, “ethyl”, “isopropyl”, “phenyl”, “cyclopropyl substituted with CN, OH and/or F”, “CH₂—O—CH₃”, “CF₃”, “CHF₂”, “pyridinyl”, “3-pyridinyl”; and R, R2, R3, R4 are all absent; 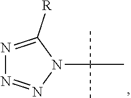
wherein R is as defined as in claim 1, but is present, such as R is “cyclopropyl”, “methyl”, “ethyl”, “isopropyl”, “phenyl”, “cyclopropyl substituted with CN, OH and/or F”, “CH₂—O—CH₃”, “CF₃”, “CHF₂”, “pyridinyl”, “3-pyridinyl”; and R1, R2, R3, R4 are all absent; 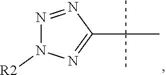
wherein R2 is as defined as in claim 1, but is present, such as R2 is “cyclopropyl”, “methyl”, “ethyl”, “isopropyl”, “phenyl”, “cyclopropyl substituted with CN, OH and/or F”, “CH₂—O—CH₃”, “CF₃”, “CHF₂”, “pyridinyl”, “3-pyridinyl”; and R, R1, R3, R4 are all absent;
or a salt thereof.

4. The compound according to claim 1,
wherein Q is Q1, Q2 or Q3, which independently are selected from the group consisting of: 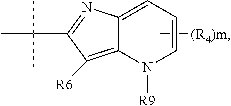
wherein R₄, R6, R9, m are as defined as in claim 1, such as R6 is “hydrogen” or “F”, R9 is “CHF₂” or “CH₂—CHF₂” or “ethyl” or “CH₂-cyclopropyl”, m is 1 and R₄ is “CF₃” or “CHF₂” or “CF₂—CH₃”; 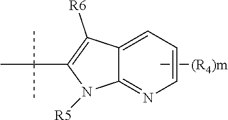
wherein R₄, R5, R6, m are as defined as in claim 1, such as R5 is “hydrogen” or “methyl”, m is 1 and R₄ is “CF₂CF₃” or “CHF₂” or “CF₂—CH₃” and R6 is “hydrogen”; 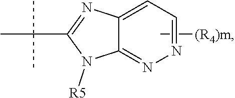
wherein R₄, R5, m are as defined as in claim 1, such as R5 is “methyl”, m is 1 and R₄ is “CHF₂” or “CF₂-cyclopropyl” or “CF₂—CF₃” or “CF₂—CH₂—CH₃” or “CF₃” or “S—CF₃” or “S(O)₂—CF₃” or “O—CH₂—CF₃” or “CF₂—CH₃”; 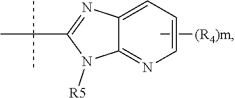
wherein R₄, R5, m are as defined as in a claim 1, such as R5 is “methyl”, m is 1 and R₄ is “CHF₂” or “CF₂—CF₃” or “CF₂-cyclopropyl” or “CF₂—CH₂—CH₃” or “S(O)₂—CF₃” or “CF₂—CH₃”; 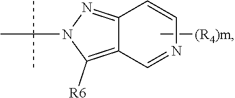
wherein R₄, R6, m are as defined as in claim 1, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃” or “CF₂—CH₃”; 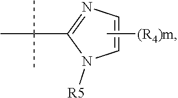
wherein R₄, R5, m are as defined as in claim 1, such as R5 is “methyl”, m is 1 and R₄ is “C(O)NH—CH₂—CF₃”;
or a salt thereof.

5. The compound according to a claim 1, wherein Q is Q1, Q2 or Q3, which independently are selected from the group consisting of: 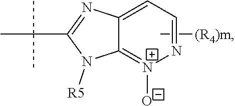
wherein R₄, R5, m are as defined as in claim 1, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃” or “CF₂—CH₃”; 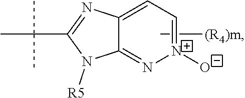
wherein R₄, R5, m are as defined as in claim 1, such as R5 is “methyl”, m is 1 and R₄ is “CHF₂” or “CF₂-cyclopropyl” or “CF₂—CF₃” or “CF₂—CH₂—CH₃” or “S—CF₃” or “S(O)₂—CF₃” or “CF₂—CH₃”; 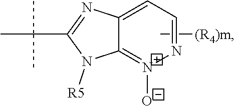
wherein R₄, R5, m are as defined as in claim 1, such as R5 is “methyl”, m is 1 and R₄ is “CHF₂” or “CF₂—CF₃” or “CF₂-cyclopropyl” or “CF₂—CH₂—CH₃” or “S(O)₂—CF₃” or “CF₂—CH₃”; 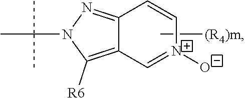
wherein R₄, R6, m are as defined as in claim 1, such as R6 is “hydrogen” or “F”, m is 1 and R₄ is “CF₃” or “CHF₂” or “CF₂—CF₃” or “S—CF₃” or “S(O)—CF₃” or “S(O)₂—CF₃”;
or a salt thereof.

6. The compound according to a claim 1, wherein optionally, if any, the at least one nitrogen atom being an N-oxide comprises the one of the central pyridine moiety selected from the group consisting of formula (II) or formula (IIa) or formula (IIb): 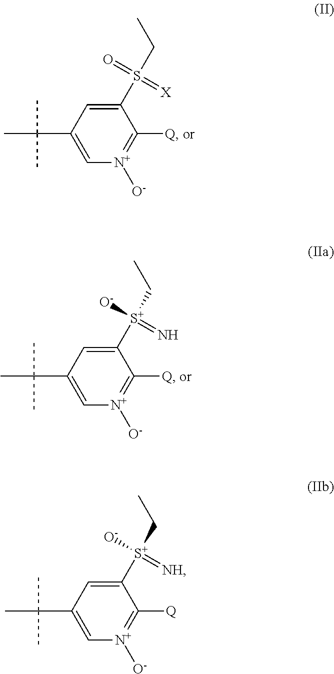
or a salt thereof,
wherein X, if present, and Q are as defined as in claim 1 and formulae (II), (IIa) and (IIb) are only part of the corresponding formulae (I), (Ia) and (Ib), respectively.

7. A compound of formula (I), (Ia) or (Ib) 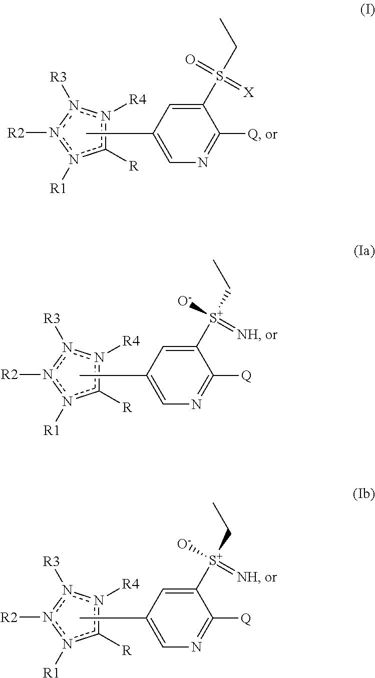
wherein:
X, if present, is independently O or NH;
and 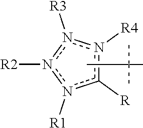
is independently selected from the group consisting of: 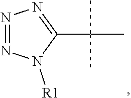
R1 is present and is independently hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃; or R1 is “cyclopropyl”, “methyl”, “ethyl”, “isopropyl”, “phenyl”, “CCHF2”, “cyclopropyl substituted with CN, OH and/or F”, “CH₂—O—CH₃”, “CF₃”, “CHF₂”, “pyridinyl”, “3-pyridinyl”; and R, R2, R3, R4 are all absent;
or 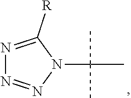
R is present and is independently hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkoxy, such as methoxy, ethoxy, propoxy, C₁-C₆-haloalkoxy, such as O—CF₃, C₁-C₆-alkyl-C₁-C₆-alkoxy, such as CH₂—O—CH₃; or R is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R1, R2, R3, R4 are all absent; 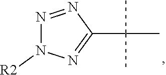
R2 is present and is independently hydrogen, C₁-C₆-alkyl, such as CH₃, CH₂—CH₃, isopropyl, C₁-C₆-haloalkyl, such as CF₃, CHF₂, CH₂—CF₃, CH₂—CHF₂, CH₂—CH₂F, CF₂—CH₃, CH₂F, CF₂—CF₃, CF₂—CHF₂, C₃-C₈-cycloalkyl, such as cyclopropyl, C₃-C₈-cycloalkyl substituted with CN, OH and/or halogen, such as F, Cl, Br, I, such as cyclopropyl substituted with CN, OH and/or F, aryl, such as phenyl, heteroaryl, such as pyridinyl, 3-pyridinyl, C₁-C₆-alkyl-C₁-C₆-alkoxy, such as CH₂—O—CH₃; or R2 is “cyclopropyl”, “methyl”, “ethyl”, “isopropyl”, “phenyl”, “CHF₂”, “cyclopropyl substituted with CN, OH and/or F”, “CH₂—O—CH₃”, “CF₃”, “CHF₂”, “pyridinyl”, “3-pyridinyl”; and R, R1, R3, R4 are all absent;
and Q is Q1, Q2 or Q3, which are independently selected from the group consisting of: 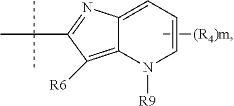
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; or R₄ is “CF₃” or “CHF₂” or “CF₂—CH₃”;
R9 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; or R9 is “CHF₂” or “CH₂—CHF₂” or “ethyl” or “CH₂-cyclopropyl”,
R6 is independently hydrogen, halogen, such as F, Cl, Br, I; or R6 is “hydrogen” or “F”;
m is 0, 1 or 2; or m is 1;
p, q in each occurrence independently are 0, 1 or 2;
or 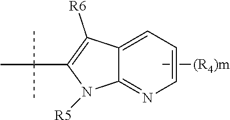
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom; or R₄ is “CF₂CF₃” or “CHF₂” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; or R5 is “hydrogen” or “methyl”;
R6 is independently hydrogen, halogen, such as F, Cl, Br, I; or R6 is “hydrogen”;
m is 0, 1 or 2; or m is 1;
p, q in each occurrence independently are 0, 1 or 2;
or 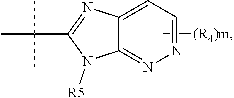
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “CHF₂” or “CF₂-cyclopropyl” or “CF₂—CF₃” or “CF₂—CH₂—CH₃” or “CF₃” or “S—CF₃” or “S(O)₂—CF₃” or “O—CH₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; or R5 is “methyl”;
m is 0, 1 or 2; or m is 1;
p, q in each occurrence independently are 0, 1 or 2;
or 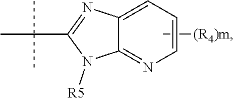
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “CHF₂” or “CF₂—CF₃” or “CF₂-cyclopropyl” or “CF₂—CH₂—CH₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃;
or R5 is “methyl”;
m is 0, 1 or 2; or m is 1;
p, q in each occurrence independently are 0, 1 or 2;
or 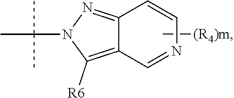
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “CF₃” or “CHF₂” or “CF₂—CF₃” or “S—CF₃” or “S(O)—CF₃” or “S(O)₂—CF₃” or “CF₂—CH₃”;
R6 is independently hydrogen, halogen, such as F, Cl, Br, I; or R6 is “hydrogen” or “F”;
m is 0, 1 or 2; or m is 1;
p, q in each occurrence independently are 0, 1 or 2;
or 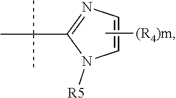
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “C(O)NH—CH₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; or R5 is “methyl”;
m is 0, 1 or 2; or m is 1;
p, q in each occurrence independently are 0, 1 or 2;
or 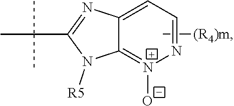
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “CHF₂” or “CF₂-cyclopropyl” or “CF₂—CF₃” or “CF₂—CH₂—CH₃” or “S—CF₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; or R5 is “methyl”;
m is 0, 1 or 2; or m is 1;
p, q in each occurrence independently are 0, 1 or 2;
or 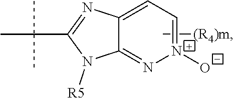
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “CHF₂” or “CF₂-cyclopropyl” or “CF₂—CF₃” or “CF₂—CH₂—CH₃” or “S—CF₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃;
or R5 is “methyl”;
m is 0, 1 or 2; or m is 1;
p, q in each occurrence independently are 0, 1 or 2;
or 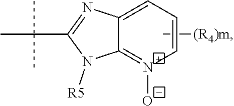
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atom, such as being “CHF₂” or “CF₂—CF₃” or “CF₂-cyclopropyl” or “CF₂—CH₂—CH₃” or “S(O)₂—CF₃” or “CF₂—CH₃”;
R5 is independently hydrogen, C₁-C₆-alkyl, such as methyl, ethyl, isopropyl, C₂-C₆-alkenyl, such as ethenyl, C₁-C₆-haloalkyl, such as CF₃, CH₂—CHF₂, CH₂—CF₃, CHF₂, C₃-C₈-cycloalkyl, such as cyclopropyl, C₃-C₈-cycloalkyl-alkyl, such as cyclopropyl-methyl, C₃-C₈-cycloalkyl substituted with one or more haloalkyl, such as cyclopropyl substituted with one or more CF₃, C₃-C₈-cycloalkyl substituted with one or more halogens, such as cyclopropyl substituted with one or more F, C(O)—(C₁-C₆-alkyl), such as C(O)-methyl, heterocyclyl, such as aziridinyl, oxetanyl, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃; or R5 is “methyl”;
m is 0, 1 or 2; or m is 1;
p, q in each occurrence independently are 0, 1 or 2;
or 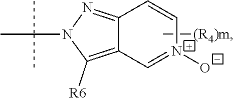
wherein:
R₄ is independently C₁-C₆-haloalkyl, such as CF₃, C₂F₅, CHF₂, C₂-C₆-haloalkenyl, such as CH═CF—CF₃, C₁-C₆-haloalkoxy, such as O—CH₂—CF₃, O—CH₂-C₂F₅, S(O)ₚ—(C₁-C₆-alkyl), such as S-methyl, S(O)-methyl; S(O)₂-methyl; S(O)_{q}—(C₁-C₆-haloalkyl), such as S—CF₃, S(O)—CF₃, S(O)₂—CF₃, C(O)NH—(C₁-C₆-alkyl), such as C(O)NH—CH₂CH₃, C(O)NH—(C₁-C₆-haloalkyl), such as C(O)NH—CH₂CF₃, C₁-C₆-haloalkyl-cyclopropyl, such as CF₂-cyclopropyl, ═O, wherein R₄, if present once or more times, in each occurrence is independently attached to a C atoms, such as being “CF₃” or “CHF₂” or “CF₂—CF₃” or “S—CF₃” or “S(O)—CF₃” or “S(O)₂—CF₃” or “CF₂—CH₃”;
R6 is independently hydrogen, halogen, such as F, Cl, Br, I; or R6 is “hydrogen” or “F”;
m is 0, 1 or 2; or m is 1;
p, q in each occurrence independently are 0, 1 or 2;
and/or
wherein optionally, if any, the at least one nitrogen atom being an N-oxide comprises the one of the central pyridine moiety selected from the group consisting of formula (II) or formula (IIa) or formula (IIb): 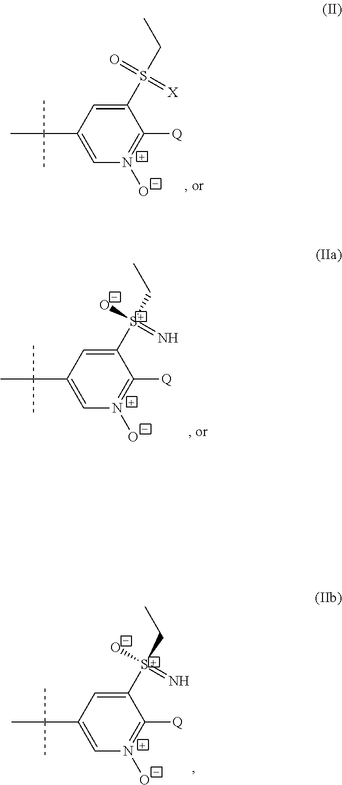
wherein X, if present, is independently O or NH; and formulae (II), (IIa) and (IIb) are only part of the corresponding formulae (I), (Ia) and (Ib), respectively;
and
wherein the dashed bond ( [symbol] ) signifies a single or double bond;
optionally, wherein at least one nitrogen atom is an N-oxide,
optionally, in case at least one nitrogen atom is an N-oxide, with the first proviso that no nitrogen atom in the four nitrogen atoms containing five-membered ring system is an N-oxide;
optionally, in case at least one nitrogen atom is an N-oxide, with the second proviso that if X is present and is NH, the NH nitrogen atom is not an N-oxide;
optionally, in case at least one nitrogen atom is an N-oxide, with the third proviso that, if possible, the at least one nitrogen atom being an N-oxide is not present in any of the radicals R, R1, R2, R3, R4;
optionally, in case at least one nitrogen atom is an N-oxide, with the fourth proviso that, if possible, and if any of Q being Q1, Q2 or Q3 is substituted, the at least one nitrogen atom being an N-oxide is not present in any potential substituents of any of Q being Q1, Q2 or Q3;
optionally, in case at least one nitrogen atom is an N-oxide, the at least one nitrogen atom being an N-oxide comprises at least one nitrogen atom within a six-membered heterocyclic moiety containing one or more nitrogen atoms of any one of Q being Q1, Q2 or Q3;
or a salt thereof.

8. A compound selected from the group consisting of: 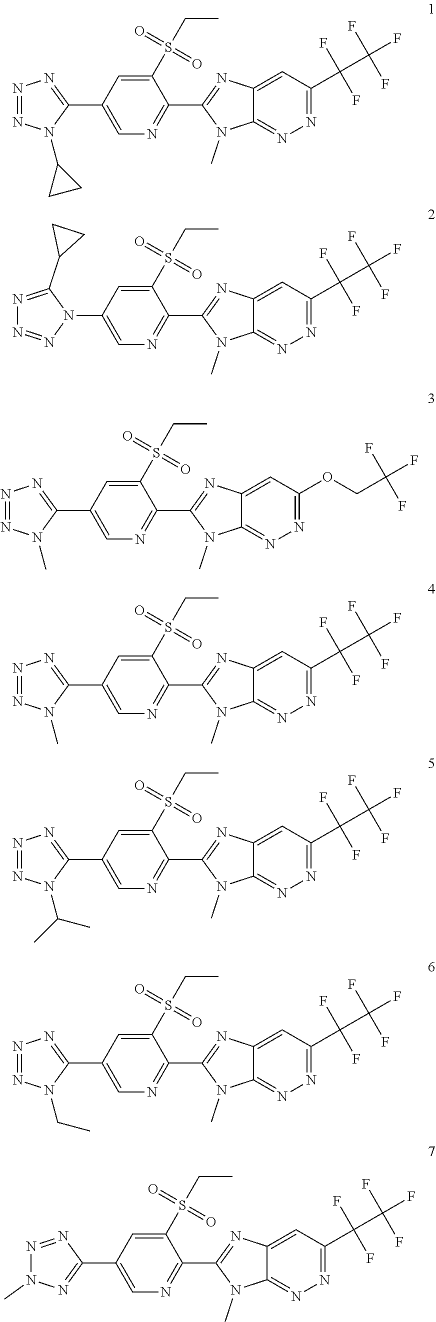 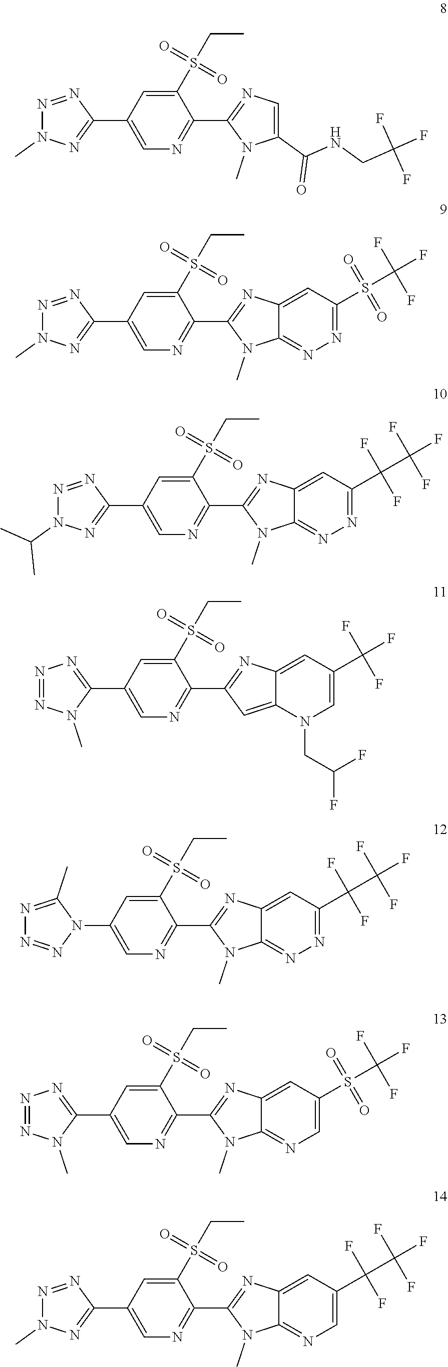 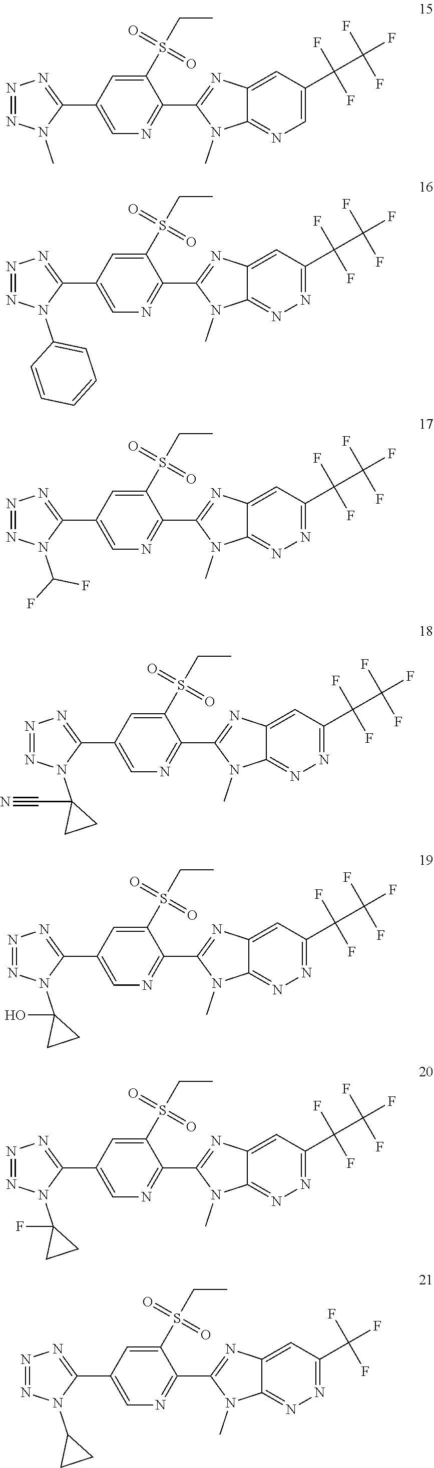 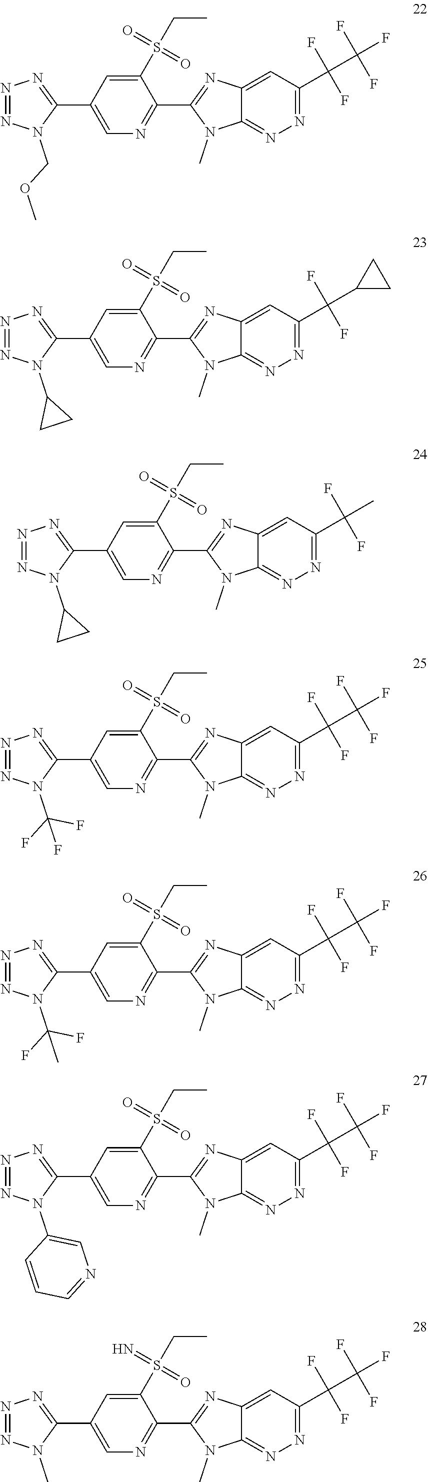 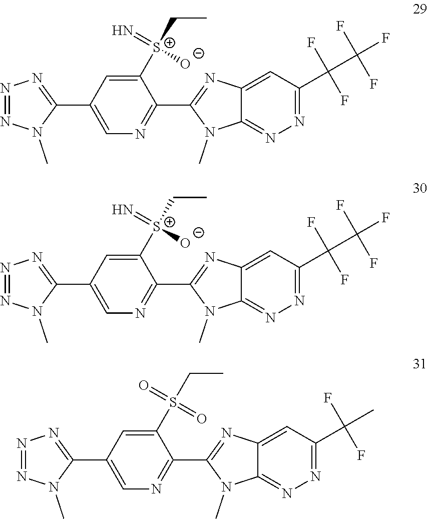
or a pharmaceutically acceptable salt thereof.

9. The compound according to claim 8 selected from the group consisting of: 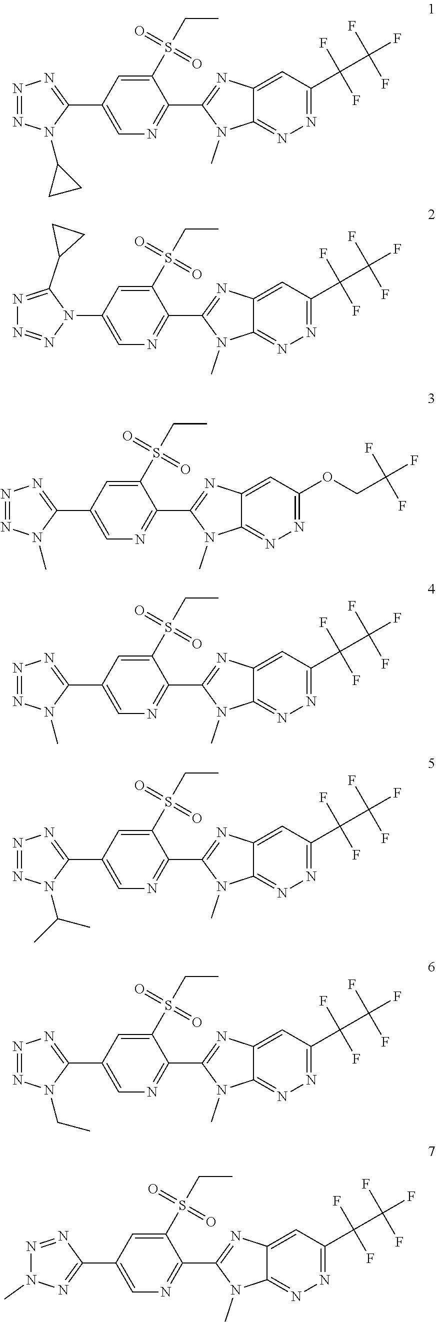 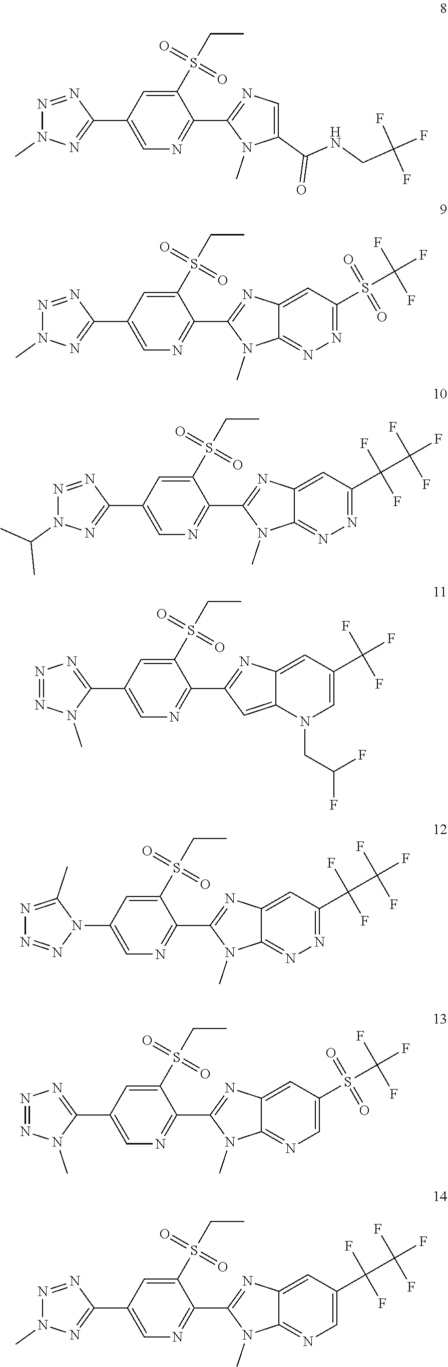 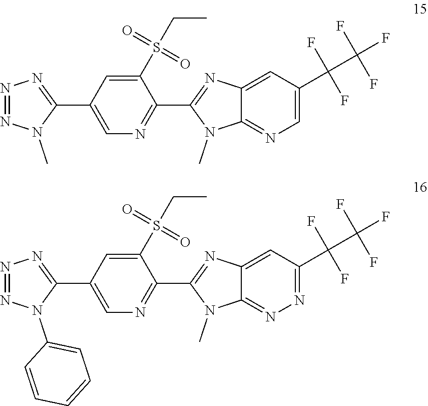
or a pharmaceutically acceptable salt thereof.

10. A pharmaceutical composition comprising one or more compound(s) of formula (I), (Ia) and/or (Ib) according to claim 1 or a pharmaceutically acceptable salt thereof and one or more pharmaceutically acceptable excipient(s).

11. A pharmaceutical composition comprising one or more compound(s) of formula (I), (Ia) and/or (Ib) according to claim 1 or a pharmaceutically acceptable salt thereof, one or more additional pharmaceutically active agent(s), and one or more pharmaceutically acceptable excipient(s).

12. An antiparasitic medicament comprising the compound of formula (I) (Ia) or (Ib) according to claim 1 or a pharmaceutically acceptable salt thereof.

13. A method of treatment, prevention and/or control of a parasitic infection and/or infestation in/on an animal, or of an ectoparasitic infestation on an animal, or of an infestation of fleas and/or ticks on an animal, the method comprising administering to the animal the compound of formula (I), (Ia) or (Ib) according to claim 1 or a pharmaceutically acceptable salt thereof.
